(19) United States
(12) Patent Application Publication
(10) Pub. No.: US 2025/0040977 A1
(43) Pub. Date: Feb. 6, 2025

(54) SYSTEMS AND METHODS FOR SELECTING, ACTIVATING, OR SELECTING AND ACTIVATING TRANSDUCERS

(71) Applicant: KARDIUM INC., Burnaby (CA)

(72) Inventors: Daniel Martin REINDERS, Surrey (CA); Justin Aaron MICHAEL, Vancouver (CA)

(21) Appl. No.: 18/905,250

(22) Filed: Oct. 3, 2024

Related U.S. Application Data
(63) Continuation of application No. PCT/CA2023/050571, filed on Apr. 27, 2023.
(60) Provisional application No. 63/336,003, filed on Apr. 28, 2022.

(51) Int. Cl. A61B 18/00; A61B 34/00; G16H 20/40

(52) U.S. Cl. CPC A61B 18/00; A61B 34/25; G16H 20/40; A61B 2018/0016; A61B 2018/00577; A61B 2018/00708

(57) ABSTRACT

A graphical representation may include a representation of a bodily cavity and graphical elements corresponding to transducers positionable in the bodily cavity. User input may be received indicating an encircling path around at least a region of the representation of at least a portion of the bodily cavity. A first region of space may be determined as being in a determined positional relationship with respect to the encircling path, the determined positional relationship being interior or exterior of the encircling path. A machine-based selection of a first transducer set may be made, the machine-based selection selecting each transducer in the first transducer set as having at least part of a corresponding graphical element in the determined first region of space. Each transducer in the first transducer set may be activated.

### CROSS-REFERENCE TO RELATED APPLICATIONS

This application is a Bypass Continuation Application of International Application No. PCT/CA2023/050571, filed Apr. 27, 2023, which claims the benefit of U.S. Provisional Application No. 63/336,003, filed Apr. 28, 2022, the entire disclosure of each of the applications cited in this sentence is hereby incorporated herein by reference.

### TECHNICAL FIELD

Aspects of this disclosure generally are related to systems and methods for selecting, activating, or selecting and activating transducers, such systems and methods applicable to, among other things, medical systems.

### BACKGROUND

Cardiac surgery was initially undertaken using highly invasive open procedures. A sternotomy, which is a type of incision in the center of the chest that separates the sternum was typically employed to allow access to the heart. In the past several decades, more and more cardiac operations are performed using intravascular or percutaneous techniques, where access to inner organs or other tissue is gained via a catheter.

Intravascular or percutaneous surgeries benefit patients by reducing surgery risk, complications and recovery time. However, the use of intravascular or percutaneous technologies also raises some particular challenges. Medical devices used in intravascular or percutaneous surgery need to be deployed via catheter systems which significantly increase the complexity of the device structure. As well, doctors do not have direct visual contact with the medical devices once the devices are positioned within the body.

One example of where intravascular or percutaneous medical techniques have been employed is in the treatment of a heart disorder called atrial fibrillation. Atrial fibrillation is a disorder in which spurious electrical signals cause an irregular heartbeat. Atrial fibrillation has been treated with various methods including a technique known as the “PV (pulmonary vein) isolation”. Research has shown that atrial fibrillation typically begins in the pulmonary veins or at the point where they attach to the left atrium. There are typically four major pulmonary veins, and some or all may be a focal point for activity that may cause atrial fibrillation. During this procedure, physicians create specific patterns of lesions in the left or right atrium to block various paths taken by the spurious electrical signals. The patterns of lesions may include a pattern of one or more lesions that encircle at least one of the pulmonary veins. Lesions were originally created using incisions, but are now typically created by ablating the tissue with various techniques including pulsed field ablation (PFA) (also known as irreversible electroporation), radio-frequency (RF) ablation, microwave ablation, laser ablation, and cryogenic ablation. Lesion formation may be performed with a high success rate under the direct vision that is provided in open procedures, but is relatively complex to perform intravascularly or percutaneously because of the difficulty in creating the lesions in the correct locations. Various problems, potentially leading to severe adverse events, may occur if the lesions are placed incorrectly. It is particularly important to know the position of the various transducers which will be creating the lesions relative to cardiac features such as the pulmonary veins and mitral valve. The continuity, transmurality and placement of the lesion patterns that are formed can impact the ability to block paths taken within the heart by spurious electrical signals. Other requirements for various ones of the transducers to perform additional functions such as, but not limited to, mapping various anatomical features, mapping electrophysiological activity, sensing tissue characteristics such as impedance and temperature and tissue stimulation can also complicate the operation of the employed medical device.

In this regard, there is a need for improved intra-bodily-cavity transducer-based device systems or control mechanisms thereof with improved transducer selection capabilities.

In this regard, there is a need for improved intra-bodily-cavity transducer-based device systems or control mechanisms thereof with enhanced tissue ablation path generation capabilities.

In this regard, there is a need for improved intra-bodily-cavity transducer-based device systems or control mechanisms thereof with enhanced transducer pattern selection capabilities to form various encircling lesions.

### SUMMARY

At least the above-discussed need is addressed, and technical solutions are achieved by various embodiments of the present invention. In some embodiments, a transducer-activation system may be summarized as including a data processing device system, an input-output device system communicatively connected to the data processing device system, and a memory device system communicatively connected to the data processing device system and storing a program executable by the data processing device system. According to some embodiments, the program may include graphical representation instructions configured to cause display, via the input-output device system, of a graphical representation including a representation of at least a portion of a bodily cavity. In some embodiments, the graphical representation may include a plurality of graphical elements corresponding to a plurality of transducers positionable in the bodily cavity. According to some embodiments, the program may include reception instructions configured to cause reception, via the input-output device system, of user input indicating an encircling path around at least a region of the representation of the at least the portion of the bodily cavity. In some embodiments, the program may include determination instructions configured to cause a determination of a first region of space in a determined positional relationship with respect to the encircling path, the determined positional relationship being interior of the encircling path or the determined positional relationship being exterior of the encircling path. According to some embodiments, the program may include selection instructions configured to cause, in response to reception of the user input indicating the encircling path and in response to the determination of the first region of space in the determined positional relationship with respect to the encircling path, a machine-based selection of a first transducer set of the plurality of transducers. In some embodiments, the machine-based selection may select each transducer in the first transducer set as having at least part of a corresponding graphical element of the displayed plurality of graphical elements in the determined first region of space. According to some embodiments, the program may include activation instructions configured to cause, via the input-output device system, activation of selected transducers of the plurality of transducers including each transducer in the first transducer set.

In some embodiments, the graphical representation instructions may be configured to cause display, via the input-output device system, of the encircling path among the at least some of the displayed plurality of graphical elements. In some embodiments, the graphical representation instructions may be configured to cause display, via the input-output device system, of the encircling path around the at least the region of the representation of the at least the portion of the bodily cavity. In some embodiments, the graphical representation instructions may be configured to cause display, via the input-output device system, of the encircling path distinctly from the displayed plurality of graphical elements.

In some embodiments, the selection instructions may be configured to cause a selection indicating a selected graphical element set from the displayed plurality of graphical elements, the selected graphical element set corresponding to the first transducer set of the plurality of transducers. In some embodiments, the machine-based selection may include a first machine-based selection of a first graphical element of the selected graphical element set, the first graphical element corresponding to a first transducer in the first transducer set. In some embodiments, the first graphical element may be selected according to the first machine-based selection as a first particular one of the plurality of graphical elements located closest to a portion of the encircling path among all graphical elements of the plurality of graphical elements located at least in part in the first region of space. In some embodiments, the machine-based selection may include a second machine-based selection of a second graphical element of the selected graphical element set. In some embodiments, the second graphical element may be selected according to the second machine-based selection as a second particular one of the plurality of graphical elements located closest, besides the first graphical element, to the portion of the encircling path, among all graphical elements of the plurality of graphical elements that are in the first region of space and spaced from the encircling path. In some embodiments, the second graphical element may be spaced from the encircling path. In some embodiments, the second graphical element may correspond to a second transducer in the first transducer set, the second transducer other than the first transducer. In some embodiments, the first transducer and the second transducer may be adjacent transducers of the plurality of transducers. In some embodiments, the first graphical element selected according to the first machine-based selection may be spaced from the encircling path.

In some embodiments, the machine-based selection may include a first machine-based selection of a first group of graphical elements of the plurality of graphical elements, each graphical element in the first group of graphical elements of the plurality of graphical elements corresponding to a respective transducer in a first group of transducers in the first transducer set. In some embodiments, each graphical element in the first group of graphical elements of the plurality of graphical elements may be selected according to the first machine-based selection as a particular one of the plurality of graphical elements located closest to a respective portion of the encircling path among all graphical elements of the plurality of graphical elements located in or located at least in part in the first region of space. In some embodiments, the machine-based selection may include a second machine-based selection of a second group of graphical elements of the plurality of graphical elements. In some embodiments, each graphical element in the second group of graphical elements of the plurality of graphical elements may be selected according to the second machine-based selection as a particular one of the plurality of graphical elements other than any graphical element in the first group of graphical elements of the plurality of graphical elements, each graphical element in the second group of graphical elements of the plurality of graphical elements corresponding to a respective transducer in a second group of transducers in the first transducer set. In some embodiments, each graphical element in the second group of graphical elements of the plurality of graphical elements may be adjacent each of at least one graphical element in the first group of graphical elements of the plurality of graphical elements. In some embodiments, each transducer in the second group of transducers may be adjacent each of at least one other transducer in the second group of transducers.

In some embodiments, the machine-based selection may include a machine-based selection of a particular group of graphical elements of the plurality of graphical elements, each graphical element in the selected particular group of graphical elements of the plurality of graphical elements corresponding to a respective transducer in the first transducer set. In some embodiments, each graphical element of at least one particular graphical element in the selected particular group of graphical elements of the plurality of graphical elements may be adjacent each graphical element of a respective set of at least three graphical elements in the selected particular group of graphical elements of the plurality of graphical elements. In some embodiments, each graphical element in each respective set of at least three graphical elements in the selected particular group of graphical elements of the plurality of graphical elements may be adjacent each other graphical element in the respective set of at least three graphical elements in the selected particular group of graphical elements of the plurality of graphical elements. In some embodiments, at least one graphical element of the at least one particular graphical element in the selected particular group of graphical elements of the plurality of graphical elements may be located closest to a portion of the encircling path among all graphical elements of the plurality of graphical elements in the first region of space. In some embodiments, the selected particular group of graphical elements of the plurality of graphical elements may consist of some, but not all, of the graphical elements of the plurality of graphical elements in the first region of space. In some embodiments, the selected particular group of graphical elements of the plurality of graphical elements may include all of the graphical elements of the plurality of graphical elements in the first region of space.

In some embodiments, the machine-based selection may include a machine-based selection of a particular group of graphical elements of the plurality of graphical elements, each graphical element in the selected particular group of graphical elements of the plurality of graphical elements corresponding to a respective transducer in the first transducer set. In some embodiments, each graphical element in the selected particular group of graphical elements of the plurality of graphical elements may be adjacent each graphical element of a respective set of at least three graphical elements in the selected particular group of graphical elements of the plurality of graphical elements. In some embodiments, at least one graphical element in a first respective set of at least three graphical elements in the selected particular group of graphical elements of the plurality of graphical elements may be other than any graphical element in a second respective set of at least three graphical elements in the selected particular group of graphical elements of the plurality of graphical elements. In some embodiments, each graphical element in a first respective set of at least three graphical elements in the selected particular group of graphical elements of the plurality of graphical elements may be other than any graphical element in a second respective set of at least three graphical elements in the selected particular group of graphical elements of the plurality of graphical elements.

In some embodiments, the machine-based selection may include a machine-based selection of a particular group of graphical elements of the plurality of graphical elements, each graphical element in the selected particular group of graphical elements of the plurality of graphical elements corresponding to a respective transducer in the first transducer set. In some embodiments, each graphical element of at least one particular graphical element in the selected particular group of graphical elements of the plurality of graphical elements may be adjacent each graphical element of a respective set of at least five graphical elements in the selected particular group of graphical elements of the plurality of graphical elements.

In some embodiments, the encircling path may define at least three non-colincar locations in the graphical representation. In some embodiments, the activation of each transducer in the first transducer set may be configured to cause tissue ablation. In some embodiments, the user input indicating the encircling path is received according to the reception instructions at a first time, the machine-based selection occurs at a second time after the first time, and the reception instructions may be configured to cause reception, via the input-output device system, of movement information received between the first time and the second time. In some embodiments, the movement information may indicate movement of at least part of the first transducer set into a particular region of the bodily cavity corresponding to the first region of space by the second time. In some embodiments, the user input indicating the encircling path may be received according to the reception instructions in a state in which at least part of the first transducer set is in a particular region of the bodily cavity corresponding to the first region of space. In some embodiments, the determined positional relationship is not both interior and exterior of the encircling path.

According to some embodiments, the determined positional relationship is a first determined positional relationship. In some embodiments, the determination instructions may be configured to cause a determination of a second region of space in a second determined positional relationship with respect to the encircling path, the second determined positional relationship being interior of the encircling path or the second determined positional relationship being exterior of the encircling path, whichever is opposite of the first determined positional relationship. In some embodiments, the selection instructions may be configured to cause, in response to reception of the user input indicating the encircling path and in response to the determination of the second region of space in the second determined positional relationship with respect to the encircling path, a machine-based selection of a second transducer set of the plurality of transducers, the machine-based selection selecting each transducer in the second transducer set as having at least part of a corresponding graphical element of the displayed plurality of graphical elements in the determined second region of space. In some embodiments, the activation instructions may be configured to cause, via the input-output device system, activation of selected transducers of the plurality of transducers including each transducer in the first transducer set and each transducer in the second transducer set. In some embodiments, the selected transducers of the plurality of transducers may consist of some, but not all, of the plurality of transducers.

Various systems may include combinations and sub-combinations of various systems described above.

According to some embodiments, a transducer-activation system may be summarized as including a data processing device system, an input-output device system communicatively connected to the data processing device system, and a memory device system communicatively connected to the data processing device system and storing a program executable by the data processing device system. According to some embodiments, the program may include graphical representation instructions configured to cause display, via the input-output device system, of a graphical representation including a plurality of graphical elements associated with a plurality of transducers positionable in a bodily cavity. In some embodiments, the program may include reception instructions configured to cause reception, via the input-output device system, of user input indicating a selected graphical element set from the displayed plurality of graphical elements, the selected graphical element set associated with an arrangement of transducers of the plurality of transducers. In some embodiments, the arrangement of transducers is distributed around a particular region of space. In some embodiments, the program may include determination instructions configured to determine, based at least on a spatial relationship among at least a group of transducers in the arrangement of transducers, a first region of space in a determined positional relationship with respect to the arrangement of transducers, the determined positional relationship being interior of the arrangement of transducers or the determined positional relationship being exterior of the arrangement of transducers. In some embodiments, the program may include selection instructions configured to cause, in response to reception of the user input indicating the selected graphical element set and in response to the determination of the first region of space in the determined positional relationship with respect to the arrangement of transducers, a machine-based selection of a transducer set of the plurality of transducers, the machine-based selection selecting each transducer in the transducer set as being in the determined first region of space in the determined positional relationship with respect to the arrangement of transducers. According to various embodiments, no transducer in the transducer set is any transducer of the arrangement of transducers indicated by the selected graphical element set. In some embodiments, the program may include activation instructions configured to cause, via the input-output device system, activation of selected transducers including each transducer in the arrangement of transducers and each transducer in the transducer set.

In some embodiments, the activation of the selected transducers including each transducer in the arrangement of transducers and each transducer in the transducer set may be configured to cause tissue ablation. In some embodiments, the activation of the selected transducers including each transducer in the arrangement of transducers and each transducer in the transducer set may be configured to cause an ablated tissue lesion that at least partially surrounds at least part of the first region of space.

In some embodiments, the machine-based selection may include a selection of at least a first transducer in the transducer set as adjacent a particular transducer of the arrangement of transducers. In some embodiments, the machine-based selection may include a selection of multiple transducers in the transducer set, each transducer in the multiple transducers in the transducer set adjacent at least one respective transducer of the arrangement of transducers. In some embodiments, the machine-based selection may include a first machine-based selection of a first transducer in the transducer set. In some embodiments, the first transducer may be selected according to the first machine-based selection as being closest to a particular transducer in the arrangement of transducers among all transducers of the plurality of transducers located at least in part in the first region of space.

In some embodiments, the transducer set may include multiple transducers of the plurality of transducers. In some embodiments, the multiple transducers of the plurality of transducers may be distributed around at least part of the first region of space. In some embodiments, the multiple transducers of the plurality of transducers may include all of the transducers of the plurality of transducers located in the first region of space. In some embodiments, the multiple transducers of the plurality of transducers may include some, but not all, of the transducers of the plurality of transducers located in the first region of space.

In some embodiments, the user input may include a user-based selection indicating a user-selected path, and the selection instructions may be configured to cause, in response to the user-based selection, a particular machine-based selection of the selected graphical element set as along the user-selected path. In some embodiments, the graphical representation instructions may be configured to cause display of the user-selected path the selected graphical element set in the graphical representation.

In some embodiments, the selection instructions may be configured to cause a selection indicating the selected graphical element set from the displayed plurality of graphical elements, the selected graphical element set corresponding to the transducer set of the plurality of transducers. In some embodiments, the arrangement of transducers may include at least three transducers of the plurality of transducers that are not colinearly arranged with respect to one another. In some embodiments, the group of transducers in the arrangement of transducers may include at least three transducers of the plurality of transducers that are not colinearly arranged with respect to one another. In some embodiments, the determined positional relationship is not both interior and exterior of the arrangement of transducers.

Various systems may include combinations and sub-combinations of various systems described above.

According to some embodiments, a transducer-activation system may be summarized as including a data processing device system, an input-output device system communicatively connected to the data processing device system, and a memory device system communicatively connected to the data processing device system and storing a program executable by the data processing device system. In some embodiments, the program includes graphical representation instructions configured to cause display, via the input-output device system, of a graphical representation including a plurality of graphical elements corresponding to a plurality of transducers positionable in a bodily cavity. In some embodiments, the program may include reception instructions configured to cause reception, via the input-output device system, of user input indicating at least a first encircling path among a first group of at least some of the displayed plurality of graphical elements and a second encircling path among a second group of at least some of the displayed plurality of graphical elements, at least a portion of the second encircling path located interior of the first encircling path. In some embodiments, the program may include determination instructions configured to cause a determination of a first region of space interior of the first encircling path and exterior of the at least the portion of the second encircling path. In some embodiments, the program may include selection instructions configured to cause, in response to reception of the user input indicating the first encircling path and the second encircling path, and in response to the determination of the first region of space, a machine-based selection of a first transducer set of the plurality of transducers, the machine-based selection selecting each transducer in the first transducer set as having at least part of a corresponding graphical element of the displayed plurality of graphical elements in the determined first region of space. In some embodiments, the program may include activation instructions configured to cause, via the input-output device system, activation of selected transducers of the plurality of transducers including each transducer in the first transducer set.

In some embodiments, the graphical representation instructions may be configured to cause display, via the input-output device system, of (a) the first encircling path among the first group of at least some of the displayed plurality of graphical elements, (b) the second encircling path among the second group of at least some of the displayed plurality of graphical elements, or both (a) and (b). In some embodiments, the graphical representation instructions may be configured to cause display, via the input-output device system, of (a) the first encircling path distinctly from the displayed plurality of graphical elements, (b) the second encircling path distinctly from the displayed plurality of graphical elements, or both (a) and (b). In some embodiments, (a) at least a first graphical element of the plurality of graphical elements is intersected by the first encircling path, (b) at least a second graphical element of the plurality of graphical elements is intersected by the second encircling path, or both (a) and (b).

In some embodiments, the selection instructions may be configured to cause a selection indicating a selected first graphical element set from the displayed plurality of graphical elements, the selected first graphical element set corresponding to the first transducer set of the plurality of transducers. In some embodiments, the selected first graphical element set may include multiple graphical elements of the displayed plurality of graphical elements. In some embodiments, the second encircling path may encircle a second graphical element set of the plurality of graphical elements. In some embodiments, the second graphical element set may include multiple graphical elements of the displayed plurality of graphical elements.

In some embodiments, the at least the portion of the second encircling path may be the entirety of the second encircling path. In some embodiments, the corresponding graphical elements of the transducers of the first transducer set may be distributed around the second encircling path.

Various systems may include combinations and sub-combinations of various systems described above.

According to some embodiments, a transducer-activation system may be summarized as including a data processing device system, an input-output device system communicatively connected to the data processing device system, and a memory device system communicatively connected to the data processing device system and storing a program executable by the data processing device system. In some embodiments, the program may include graphical representation instructions configured to cause display, via the input-output device system, of a graphical representation including a representation of at least a portion of a bodily cavity. In some embodiments, the graphical representation may include a plurality of graphical elements corresponding to a plurality of transducers positionable in the bodily cavity. In some embodiments, the program may include reception instructions configured to cause reception, via the input-output device system, of user input indicating an encircling path among at least a region of the representation of the at least the portion of the bodily cavity. In some embodiments, the program may include first determination instructions configured to cause a determination of a first region of space in a first determined positional relationship with respect to the encircling path, the first determined positional relationship being interior of the encircling path or the first determined positional relationship being exterior of the encircling path. In some embodiments, the program may include second determination instructions configured to cause a determination of a second region of space in a second determined positional relationship with respect to the encircling path, the second determined positional relationship being interior or exterior of the encircling path, whichever is opposite the first determined positional relationship. In some embodiments, the program may include selection instructions configured to cause, in response to reception of the user input indicating the encircling path and in response to the determinations of the first and second regions of space in the first and second determined positional relationships, respectively, with respect to the encircling path, a machine-based selection of a first transducer set of the plurality of transducers, the machine-based selection selecting the first transducer set as collectively having corresponding graphical elements of the displayed plurality of graphical elements in both the first and second regions of space. In some embodiments, the machine-based selection may select each transducer in the first transducer set as having at least part of a corresponding graphical element of the displayed plurality of graphical elements in the determined first region of space, in the determined second region of space, or in both the determined first and second regions of space. In some embodiments, the program may include activation instructions configured to cause, via the input-output device system, activation of selected transducers of the plurality of transducers including each transducer in the first transducer set.

Various embodiments of the present invention may include systems, devices, or machines that are or include combinations or subsets of any one or more of the systems, devices, or machines and associated features thereof summarized above or otherwise described herein (which should be deemed to include the figures).

Further, all or part of any one or more of the systems, devices, or machines summarized above or otherwise described herein or combinations or sub-combinations thereof may implement or execute all or part of any one or more of the processes or methods described herein or combinations or sub-combinations thereof.

In some embodiments, a method is executed by a data processing device system according to a program stored by a communicatively connected memory device system, where the data processing device system also is communicatively connected to an input-output device system. The method may include displaying, via the input-output device system, a graphical representation including a representation of at least a portion of a bodily cavity. The graphical representation may include a plurality of graphical elements corresponding to a plurality of transducers positionable in the bodily cavity. The method may include receiving, via the input-output device system, user input indicating an encircling path around at least a region of the representation of the at least the portion of the bodily cavity. The method may include determining a first region of space in a determined positional relationship with respect to the encircling path. The determined positional relationship may be interior of the encircling path, or the determined positional relationship may be exterior of the encircling path. The method may include selecting, in response to reception of the user input indicating the encircling path and in response to the determination of the first region of space in the determined positional relationship with respect to the encircling path, each transducer in a first transducer set of the plurality of transducers as having at least part of a corresponding graphical element of the displayed plurality of graphical elements in the determined first region of space. The method may include activating, via the input-output device system, selected transducers of the plurality of transducers including each transducer in the first transducer set.

In some embodiments, a method is executed by a data processing device system according to a program stored by a communicatively connected memory device system, where the data processing device system also is communicatively connected to an input-output device system. The method may include displaying, via the input-output device system, a graphical representation including a plurality of graphical elements associated with a plurality of transducers positionable in a bodily cavity. The method may include receiving, via the input-output device system, user input indicating a selected graphical element set from the displayed plurality of graphical elements. The selected graphical element set may be associated with an arrangement of transducers of the plurality of transducers, the arrangement of transducers distributed around a particular region of space. The method may include determining, based at least on a spatial relationship among at least a group of transducers in the arrangement of transducers, a first region of space in a determined positional relationship with respect to the arrangement of transducers. The determined positional relationship may be interior of the arrangement of transducers, or the determined positional relationship may be exterior of the arrangement of transducers. The method may include selecting, in response to reception of the user input indicating the selected graphical element set and in response to the determination of the first region of space in the determined positional relationship with respect to the arrangement of transducers, each transducer of a transducer set of the plurality of transducers as being in the determined first region of space in the determined positional relationship with respect to the arrangement of transducers, and no transducer in the transducer set being any transducer of the arrangement of transducers indicated by the selected graphical element set. The method may include activating, via the input-output device system, selected transducers including each transducer in the arrangement of transducers and each transducer in the transducer set.

In some embodiments, a method is executed by a data processing device system according to a program stored by a communicatively connected memory device system, where the data processing device system also is communicatively connected to an input-output device system. The method may include displaying, via the input-output device system, a graphical representation including a plurality of graphical elements corresponding to a plurality of transducers positionable in a bodily cavity. The method may include receiving, via the input-output device system, user input indicating at least a first encircling path among a first group of at least some of the displayed plurality of graphical elements and a second encircling path among a second group of at least some of the displayed plurality of graphical elements, at least a portion of the second encircling path located interior of the first encircling path. The method may include determining a first region of space interior of the first encircling path and exterior of the at least the portion of the second encircling path. The method may include selecting, in response to reception of the user input indicating the first encircling path and the second encircling path, and in response to the determination of the first region of space, each transducer of a first transducer set of the plurality of transducers as having at least part of a corresponding graphical element of the displayed plurality of graphical elements in the determined first region of space. The method may include activating, via the input-output device system, selected transducers of the plurality of transducers including each transducer in the first transducer set.

In some embodiments, a method is executed by a data processing device system according to a program stored by a communicatively connected memory device system, where the data processing device system also is communicatively connected to an input-output device system. The method may include displaying, via the input-output device system, a graphical representation including a representation of at least a portion of a bodily cavity and including a plurality of graphical elements corresponding to a plurality of transducers positionable in the bodily cavity. The method may include receiving, via the input-output device system, user input indicating an encircling path among at least a region of the representation of the at least the portion of the bodily cavity. The method may include determining a first region of space in a first determined positional relationship with respect to the encircling path. The first determined positional relationship may be interior of the encircling path, or the first determined positional relationship may be exterior of the encircling path. The method may include determining a second region of space in a second determined positional relationship with respect to the encircling path. The second determined positional relationship may be interior or exterior of the encircling path, whichever is opposite the first determined positional relationship. The method may include selecting, in response to reception of the user input indicating the encircling path and in response to the determinations of the first and second regions of space in the first and second determined positional relationships, respectively, with respect to the encircling path, a first transducer set of the plurality of transducers as collectively having corresponding graphical elements of the displayed plurality of graphical elements in both the first and second regions of space, and each transducer in the first transducer set selected by the selecting as having at least part of a corresponding graphical element of the displayed plurality of graphical elements in the determined first region of space, in the determined second region of space, or in both the determined first and second regions of space. The method may include activating, via the input-output device system, selected transducers of the plurality of transducers including each transducer in the first transducer set.

It should be noted that various embodiments of the present invention include variations of the methods or processes summarized above or otherwise described herein (which should be deemed to include the figures) and, accordingly, are not limited to the actions described or shown in the figures or their ordering, and not all actions shown or described are required according to various embodiments. According to various embodiments, such methods may include more or fewer actions and different orderings of actions. Any of the features of all or part of any one or more of the methods or processes summarized above or otherwise described herein may be combined with any of the other features of all or part of any one or more of the methods or processes summarized above or otherwise described herein.

In addition, a computer program product may be provided that includes program code portions for performing some or all of any one or more of the methods or processes and associated features thereof described herein, when the computer program product is executed by a computer or other computing device or device system. Such a computer program product may be stored on one or more computer-readable storage mediums, also referred to as one or more computer-readable data storage mediums or a computer-readable storage medium system.

In some embodiments, one or more computer-readable or non-transitory computer-readable storage mediums store a program executable by a data processing device system communicatively connected to an input-output device system. The program may include graphical representation instructions configured to cause display, via the input-output device system, of a graphical representation including a representation of at least a portion of a bodily cavity, the graphical representation including a plurality of graphical elements corresponding to a plurality of transducers positionable in the bodily cavity. The program may include reception instructions configured to cause reception, via the input-output device system, of user input indicating an encircling path around at least a region of the representation of the at least the portion of the bodily cavity. The program may include determination instructions configured to cause a determination of a first region of space in a determined positional relationship with respect to the encircling path. The determined positional relationship may be interior of the encircling path, or the determined positional relationship may be exterior of the encircling path. The program may include selection instructions configured to cause, in response to reception of the user input indicating the encircling path and in response to the determination of the first region of space in the determined positional relationship with respect to the encircling path, a machine-based selection of a first transducer set of the plurality of transducers, the machine-based selection selecting each transducer in the first transducer set as having at least part of a corresponding graphical element of the displayed plurality of graphical elements in the determined first region of space. The program may include activation instructions configured to cause, via the input-output device system, activation of selected transducers of the plurality of transducers including each transducer in the first transducer set.

In some embodiments, one or more computer-readable or non-transitory computer-readable storage mediums store a program executable by a data processing device system communicatively connected to an input-output device system. The program may include graphical representation instructions configured to cause display, via the input-output device system, of a graphical representation including a plurality of graphical elements associated with a plurality of transducers positionable in a bodily cavity. The program may include reception instructions configured to cause reception, via the input-output device system, of user input indicating a selected graphical element set from the displayed plurality of graphical elements. The selected graphical element set may be associated with an arrangement of transducers of the plurality of transducers. The arrangement of transducers may be distributed around a particular region of space. The program may include determination instructions configured to cause determination, based at least on a spatial relationship among at least a group of transducers in the arrangement of transducers, a first region of space in a determined positional relationship with respect to the arrangement of transducers. The determined positional relationship may be interior of the arrangement of transducers, or the determined positional relationship may be exterior of the arrangement of transducers. The program may include selection instructions configured to cause, in response to reception of the user input indicating the selected graphical element set and in response to the determination of the first region of space in the determined positional relationship with respect to the arrangement of transducers, a machine-based selection of a transducer set of the plurality of transducers, the machine-based selection selecting each transducer in the transducer set as being in the determined first region of space in the determined positional relationship with respect to the arrangement of transducers, and no transducer in the transducer set being any transducer of the arrangement of transducers indicated by the selected graphical element set. The program may include activation instructions configured to cause, via the input-output device system, activation of selected transducers including each transducer in the arrangement of transducers and each transducer in the transducer set.

In some embodiments, one or more computer-readable or non-transitory computer-readable storage mediums store a program executable by a data processing device system communicatively connected to an input-output device system. The program may include graphical representation instructions configured to cause display, via the input-output device system, of a graphical representation including a plurality of graphical elements corresponding to a plurality of transducers positionable in a bodily cavity. The program may include reception instructions configured to cause reception, via the input-output device system, of user input indicating at least a first encircling path among a first group of at least some of the displayed plurality of graphical elements and a second encircling path among a second group of at least some of the displayed plurality of graphical elements. At least a portion of the second encircling path may be located interior of the first encircling path. The program may include determination instructions configured to cause a determination of a first region of space interior of the first encircling path and exterior of the at least the portion of the second encircling path. The program may include selection instructions configured to cause, in response to reception of the user input indicating the first encircling path and the second encircling path, and in response to the determination of the first region of space, a machine-based selection of a first transducer set of the plurality of transducers, the machine-based selection selecting each transducer in the first transducer set as having at least part of a corresponding graphical element of the displayed plurality of graphical elements in the determined first region of space. The program may include activation instructions configured to cause, via the input-output device system, activation of selected transducers of the plurality of transducers including each transducer in the first transducer set.

In some embodiments, one or more computer-readable or non-transitory computer-readable storage mediums store a program executable by a data processing device system communicatively connected to an input-output device system. The program may include graphical representation instructions configured to cause display, via the input-output device system, of a graphical representation including a representation of at least a portion of a bodily cavity and including a plurality of graphical elements corresponding to a plurality of transducers positionable in the bodily cavity. The program may include reception instructions configured to cause reception, via the input-output device system, of user input indicating an encircling path among at least a region of the representation of the at least the portion of the bodily cavity. The program may include first determination instructions configured to cause a determination of a first region of space in a first determined positional relationship with respect to the encircling path. The first determined positional relationship may be interior of the encircling path, or the first determined positional relationship may be exterior of the encircling path. The program may include second determination instructions configured to cause a determination of a second region of space in a second determined positional relationship with respect to the encircling path. The second determined positional relationship may be interior or exterior of the encircling path, whichever is opposite the first determined positional relationship. The program may include selection instructions configured to cause, in response to reception of the user input indicating the encircling path and in response to the determinations of the first and second regions of space in the first and second determined positional relationships, respectively, with respect to the encircling path, a machine-based selection of a first transducer set of the plurality of transducers, the machine-based selection selecting the first transducer set as collectively having corresponding graphical elements of the displayed plurality of graphical elements in both the first and second regions of space, and the machine-based selection selecting each transducer in the first transducer set as having at least part of a corresponding graphical element of the displayed plurality of graphical elements in the determined first region of space, in the determined second region of space, or in both the determined first and second regions of space. The program may include activation instructions configured to cause, via the input-output device system, activation of selected transducers of the plurality of transducers including each transducer in the first transducer set.

In some embodiments, each of any of one or more or all of the computer-readable data storage mediums or medium systems (also referred to as processor-accessible memory device systems) described herein is a non-transitory computer-readable (or processor-accessible) data storage medium or medium system (or memory device system) including or consisting of one or more non-transitory computer-readable (or processor-accessible) storage mediums (or memory devices) storing the respective program(s) which may configure a data processing device system to execute some or all of any of one or more of the methods or processes described herein.

Further, any of all or part of one or more of the methods or processes and associated features thereof discussed herein may be implemented or executed on or by all or part of a device system, apparatus, or machine, such as all or a part of any of one or more of the systems, apparatuses, or machines described herein or a combination or sub-combination thereof.

### BRIEF DESCRIPTION OF THE DRAWINGS

It is to be understood that the attached drawings are for purposes of illustrating aspects of various embodiments and may include elements that are not to scale.

FIG. 1 includes a schematic representation of a transducer-activation system according to various example embodiments, the transducer-activation system including a data processing device system, an input-output device system, and a memory device system.

FIG. 2 includes a cutaway diagram of a heart showing a transducer-based device percutaneously placed in a left atrium of the heart, according to various example embodiments.

FIG. 3A includes a partially schematic representation of a medical system according to various example embodiments, the medical system including a data processing device system, an input-output device system, a memory device system, and a transducer-based device including a plurality of transducers and an expandable structure shown in a delivery or unexpanded configuration.

FIG. 3B includes a portion of the medical system of FIG. 3A as viewed from a different viewing direction.

FIG. 3C includes the representation of the medical system of FIGS. 3A and 3B with the expandable structure shown in a deployed or expanded configuration, according to some embodiments.

FIG. 3D includes a portion of the medical system of FIG. 3C as viewed from a different viewing direction, according to some embodiments.

FIG. 4 includes a schematic representation of a transducer-based device that includes a flexible circuit structure, according to various example embodiments.

FIG. 5A includes a graphical interface providing a graphical representation, according to various example embodiments, including a depiction of at least a portion of a transducer-based device including a plurality of transducer graphical elements depicted among the graphical representation.

FIG. 5B includes the graphical interface of FIG. 5A with the portion of the transducer-based device depicted as viewed from a different viewing direction than that shown in FIG. 5A, according to some embodiments.

FIG. 5C includes the graphical representation provided by the graphical interface of FIG. 5A with the addition of various between graphical elements positioned between various ones of the transducer graphical elements, according to some embodiments.

FIG. 5D includes the graphical representation provided by the graphical interface of FIG. 5C but with the portion of the transducer-based device depicted as viewed from a different viewing direction than that shown in FIG. 5C, according to some embodiments.

FIG. 5E includes the graphical representation provided by the graphical interface of FIGS. 5C and 5D depicted with one particular form of two-dimensional representation in accordance with various example embodiments.

FIG. 5F includes the graphical representation provided by the graphical interface of FIGS. 5A and 5B depicted with another particular form of two-dimensional representation in accordance with various example embodiments.

FIG. 5G includes a graphical interface providing a graphical representation, according to various example embodiments, including a depiction of at least a portion of a transducer-based device including a plurality of transducer graphical elements depicted among the graphical representation, and the depiction of the at least the portion of the transducer-based device within an envelope representing at least a portion of a bodily cavity, according to some embodiments.

FIG. 5H includes a graphical interface providing a graphical representation, according to various example embodiments, including a depiction of at least a portion of a transducer-based device including a plurality of transducer graphical elements depicted among the graphical representation, and the depiction of the at least the portion of the transducer-based device within a representation of at least a portion of a bodily cavity, the representation of the at least the portion of the bodily cavity based on or derived from a computerized tomography (“CT”) scan image, according to some embodiments.

FIG. 51 includes the graphical representation provided by the graphical interface of FIG. 5E including a representation of intra-cardiac information, according to some embodiments.

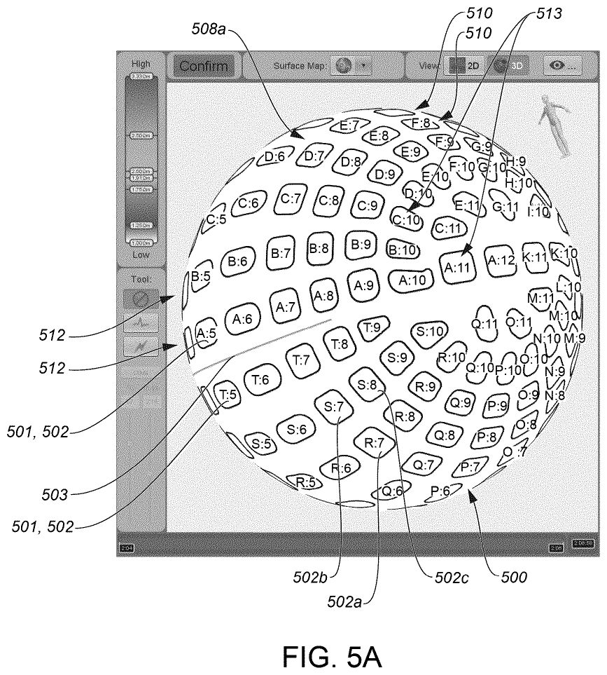

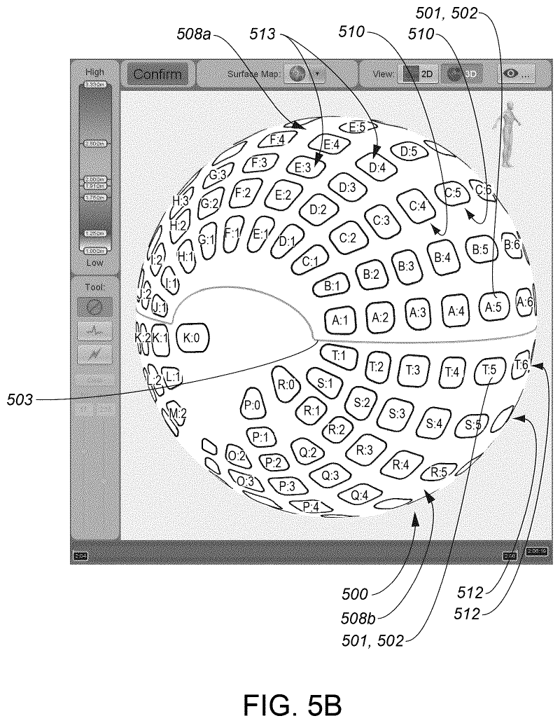

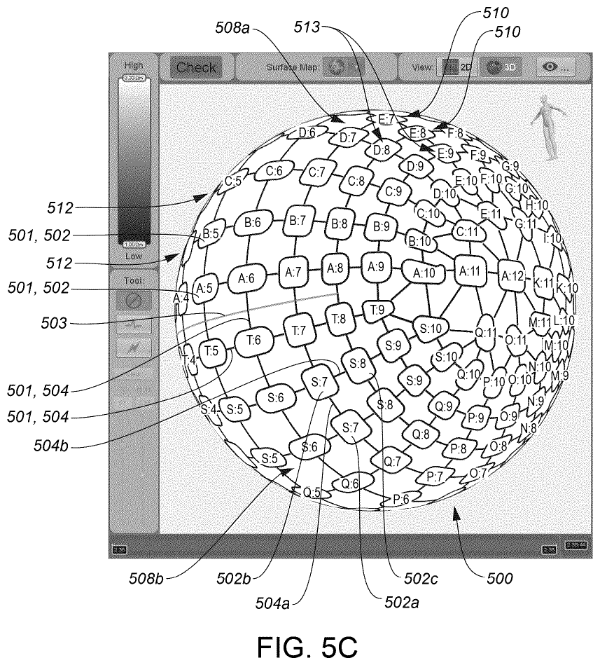

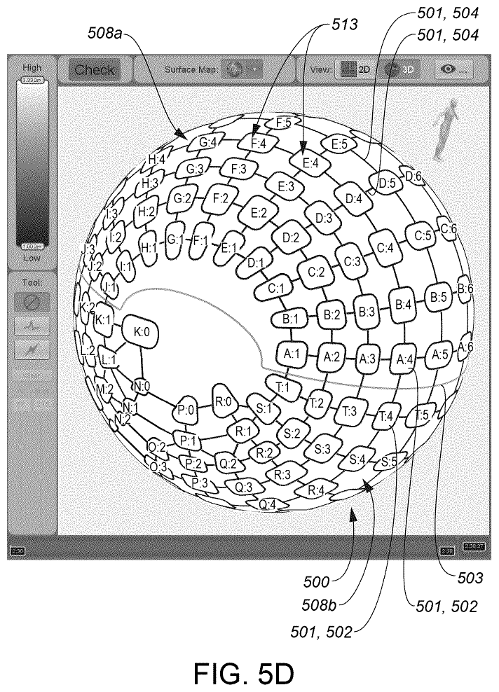

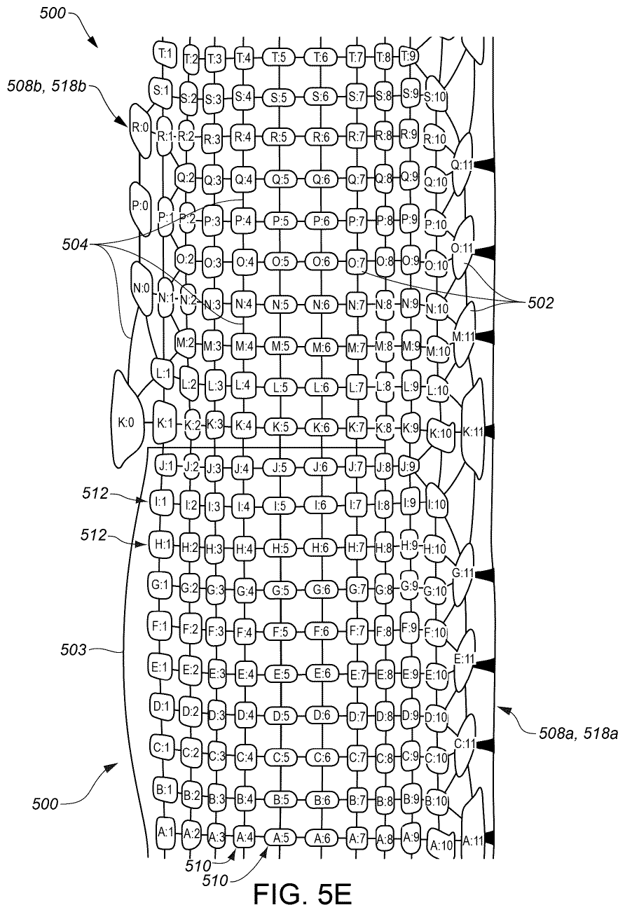

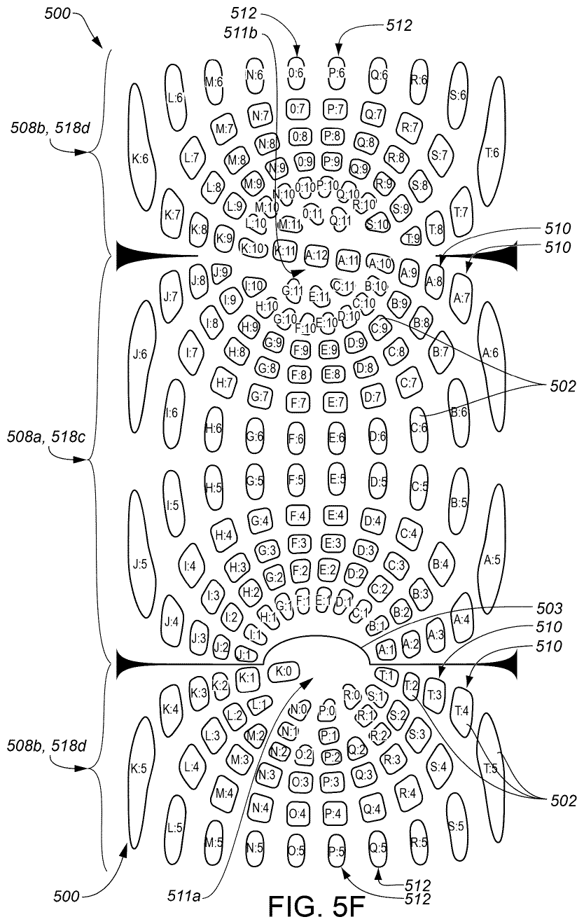

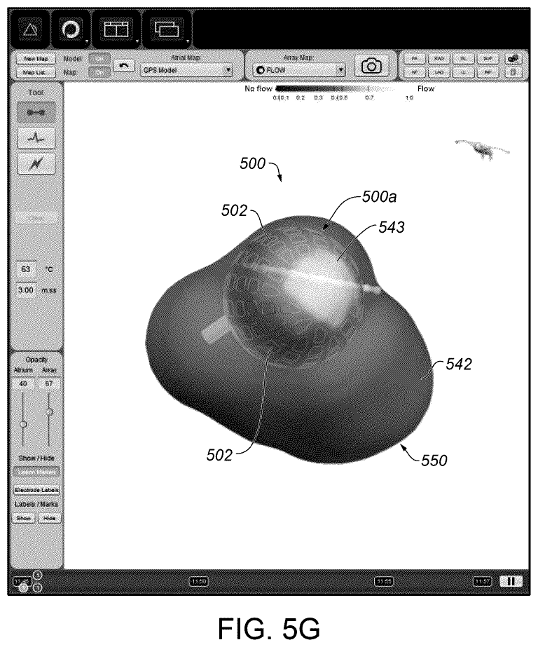

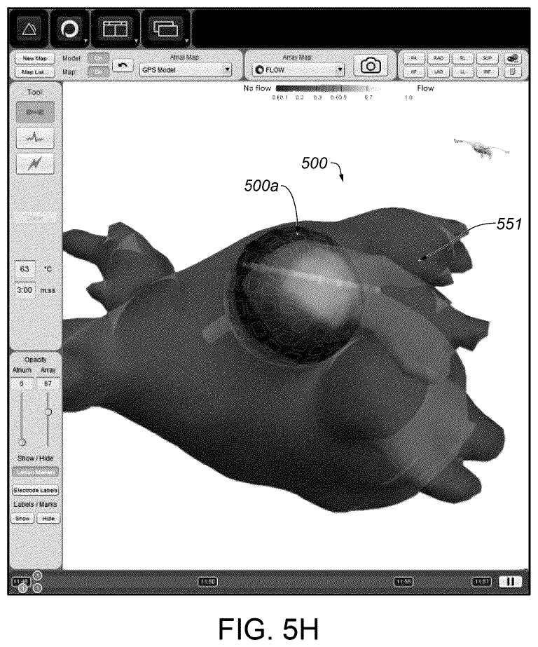

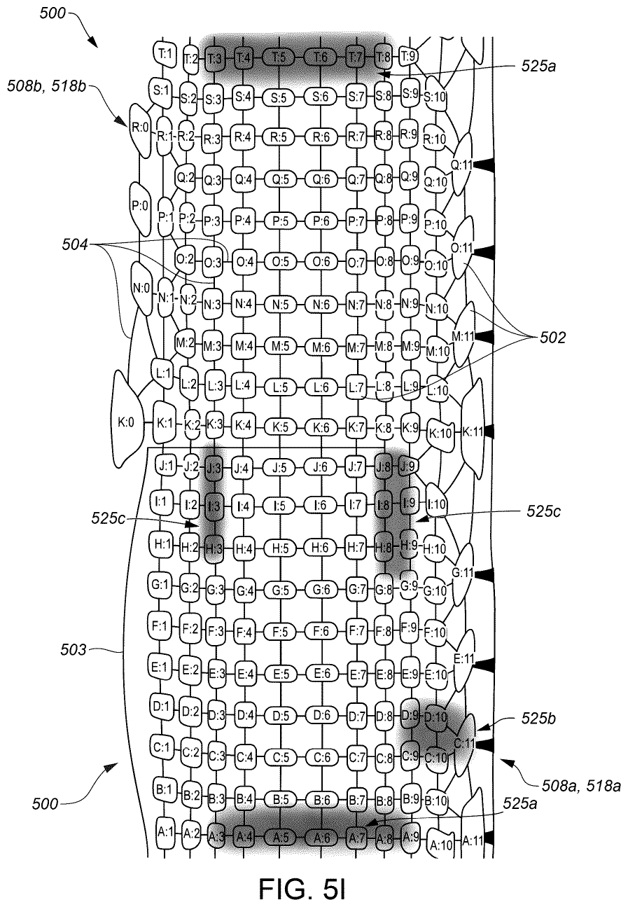

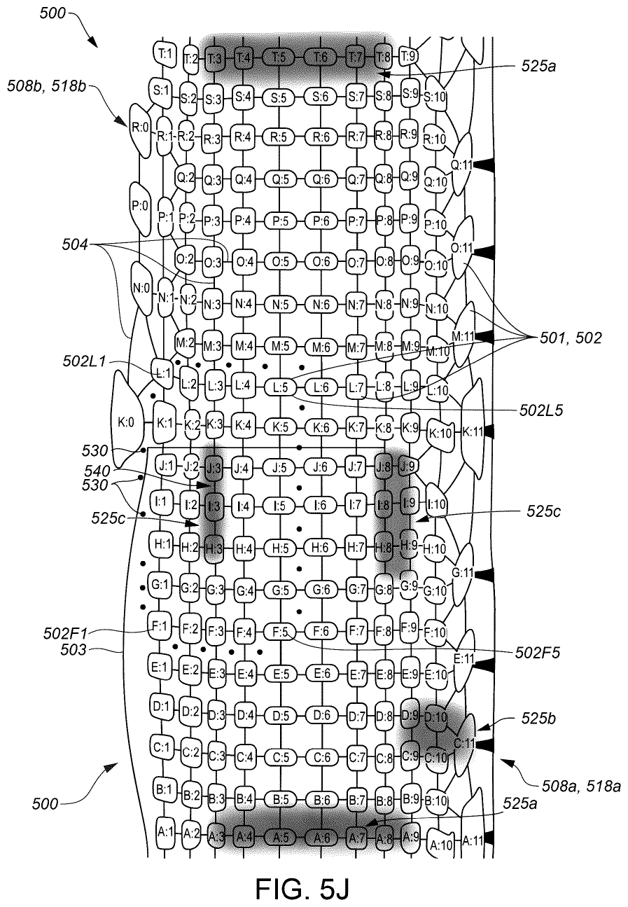

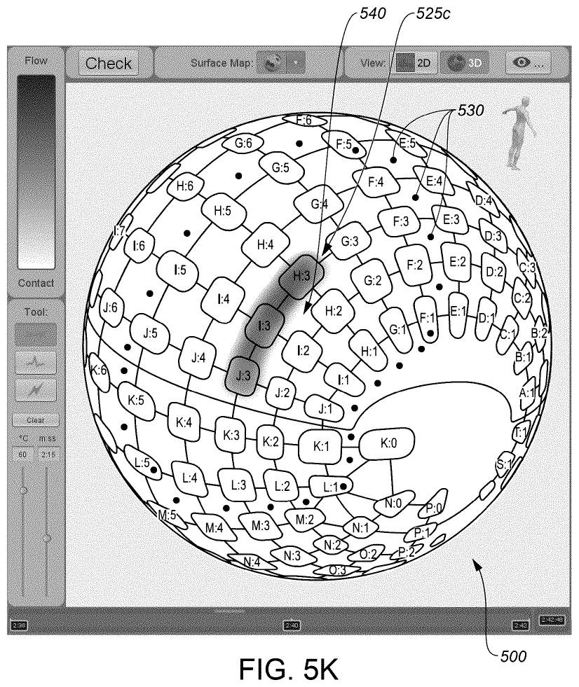

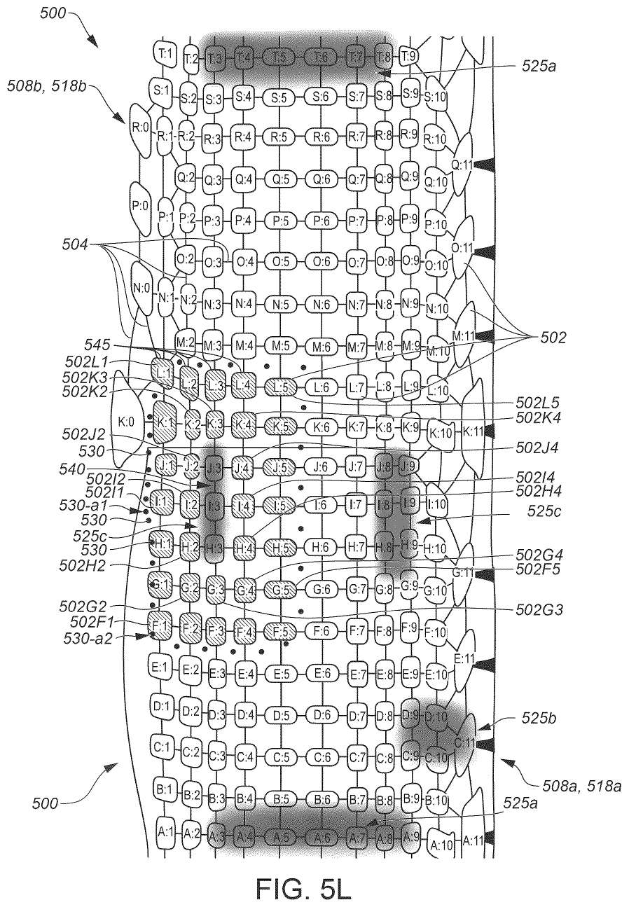

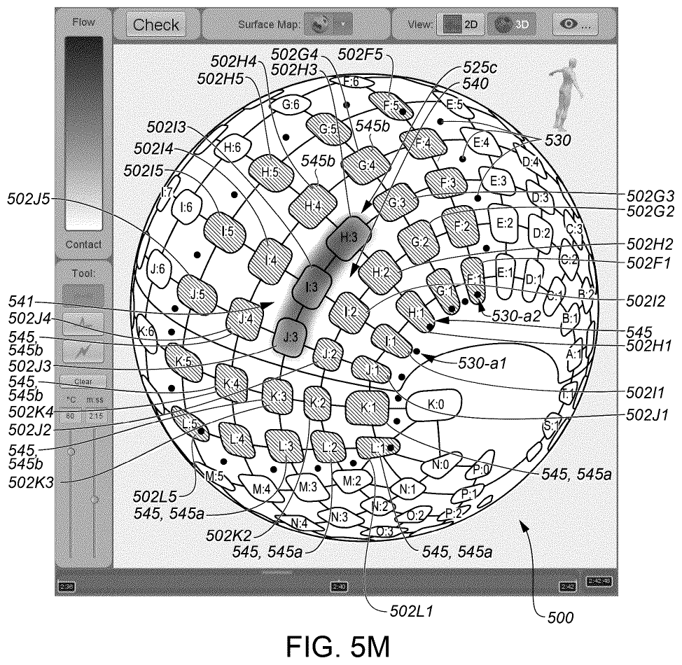

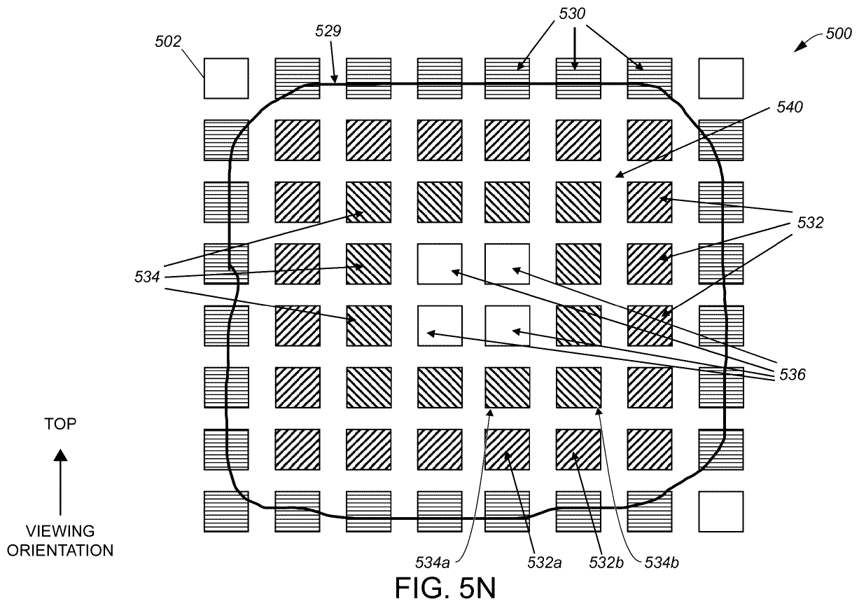

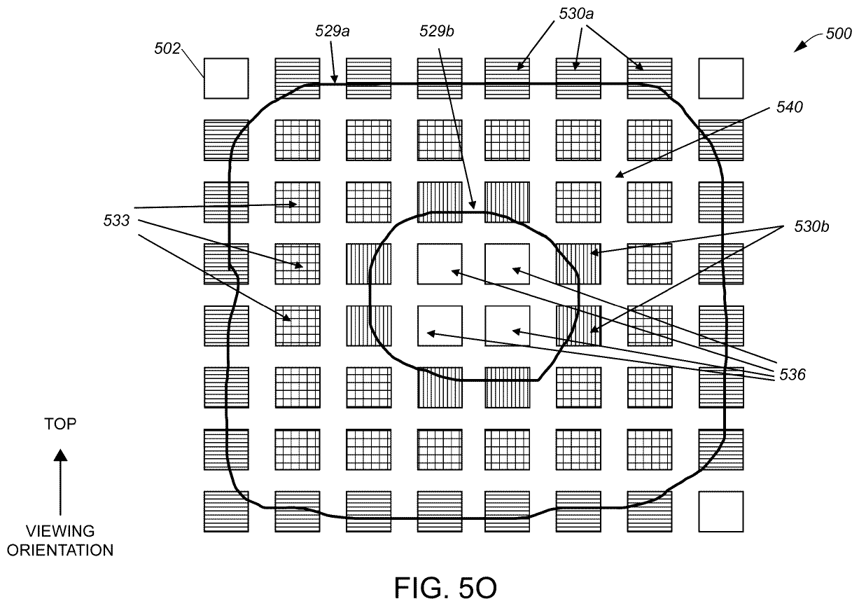

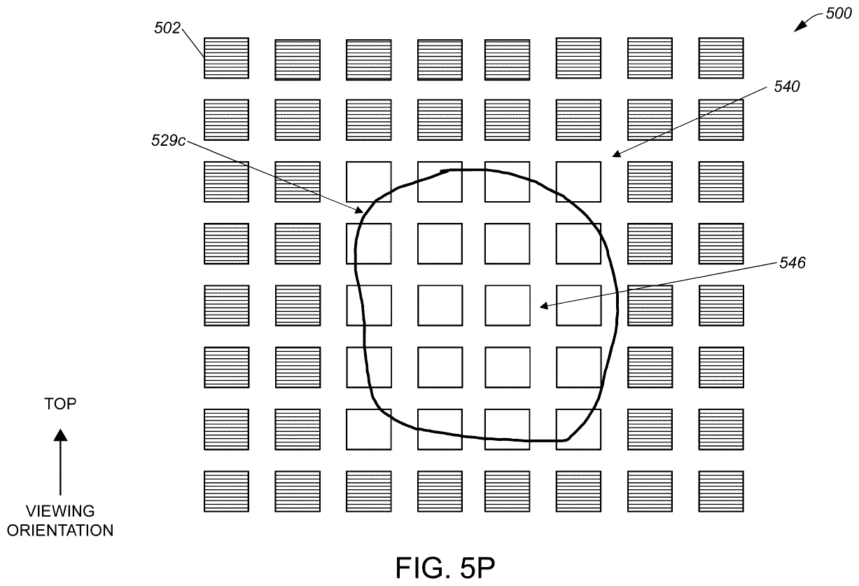

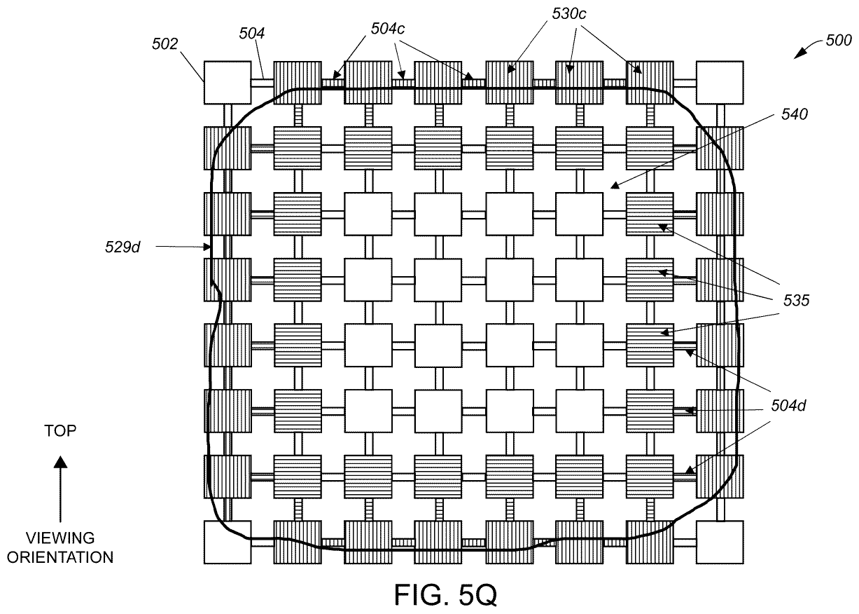

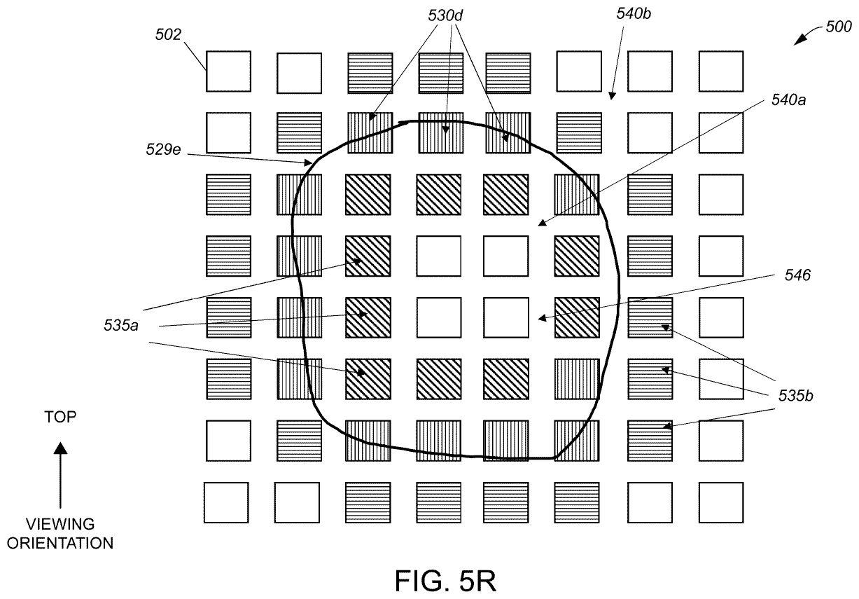

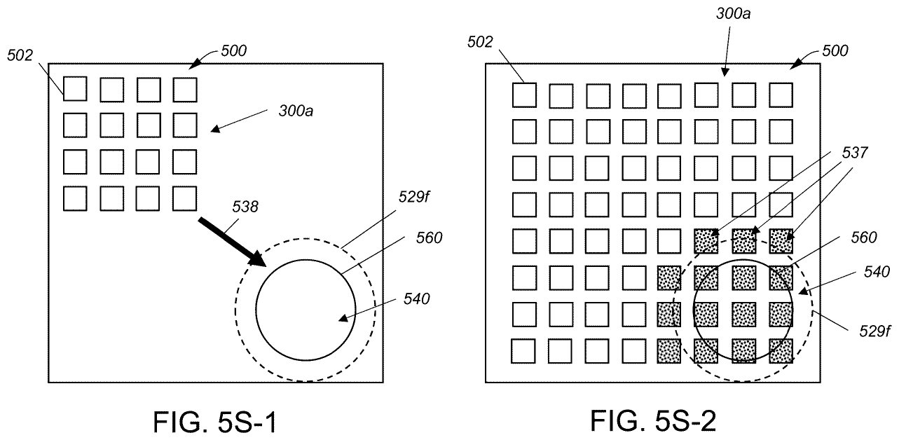

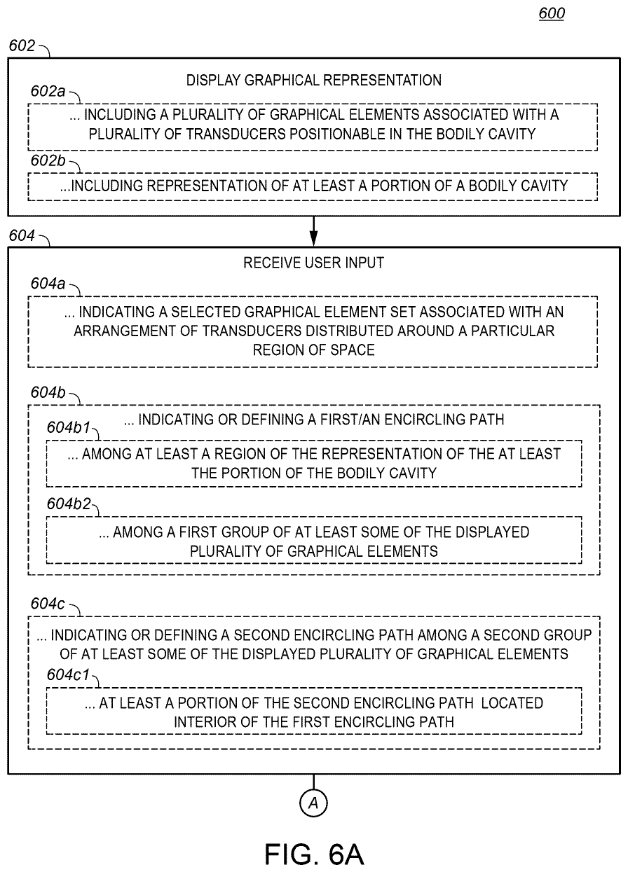

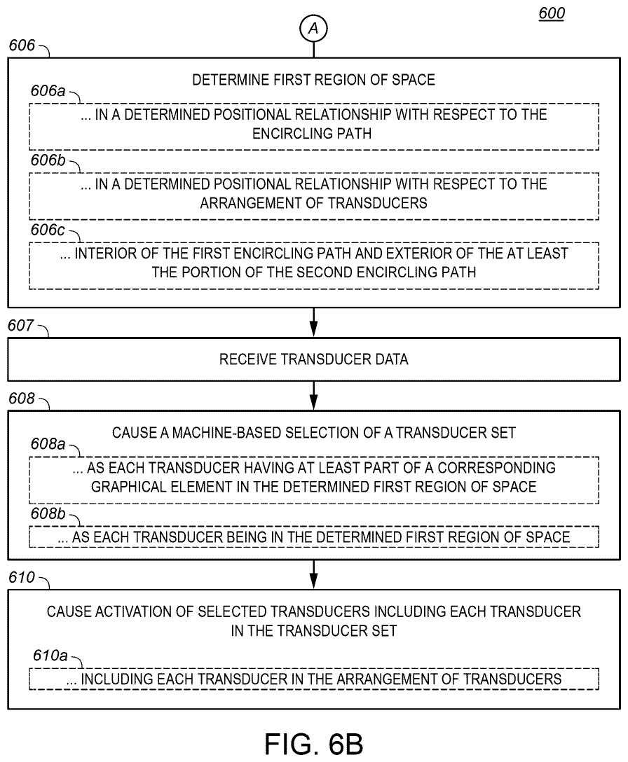

FIG. 5J includes the graphical representation shown in FIG. 5I, but further including an encircling path surrounding a region of interest depicted by the intra-cardiac information, according to some embodiments.

FIG. 5K is based on, although not identical to, the graphical representation of FIG. 5J, but with a three-dimensional perspective, according to some embodiments.

FIG. 5L is based on, although not identical to, the graphical representation of FIG. 5J, but further including selected transducer graphical elements, according to some embodiments.

FIG. 5M is based on, although not identical to, the graphical representation of FIG. 5J, but further including selected transducer graphical elements, according to some embodiments.

FIG. 5N illustrates a graphical representation of at least a portion of a transducer-based device, the graphical representation of the at least the portion of the transducer-based device including transducer graphical elements corresponding to transducers of the transducer-based device, and the graphical representation presented at least in a state in which an outer ring and an inner ring of transducer graphical elements are selected within an encircling path, according to some embodiments.

FIG. 5O illustrates a graphical representation of at least a portion of a transducer-based device, the graphical representation of the at least the portion of the transducer-based device including transducer graphical elements corresponding to transducers of the transducer-based device, and the graphical representation presented at least in a state in which transducer graphical elements are selected within between two encircling paths, according to some embodiments.

FIG. 5P illustrates a graphical representation of at least a portion of a transducer-based device, the graphical representation of the at least the portion of the transducer-based device including transducer graphical elements corresponding to transducers of the transducer-based device, and the graphical representation presented at least in a state in which transducer graphical elements are selected outside an encircling path, according to some embodiments.

FIG. 5Q illustrates a graphical representation of at least a portion of a transducer-based device, the graphical representation of the at least the portion of the transducer-based device including transducer graphical elements corresponding to transducers of the transducer-based device, and the graphical representation presented at least in a state in which transducer graphical elements and between graphical elements, which are respectively between a pair of transducer graphical elements, are selected within an encircling path, according to some embodiments.

FIG. 5R illustrates a graphical representation of at least a portion of a transducer-based device, the graphical representation of the at least the portion of the transducer-based device including transducer graphical elements corresponding to transducers of the transducer-based device, and the graphical representation presented at least in a state in which transducer graphical elements are selected within and outside an encircling path, according to some embodiments.

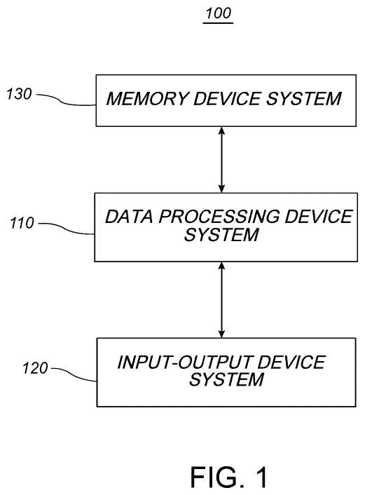

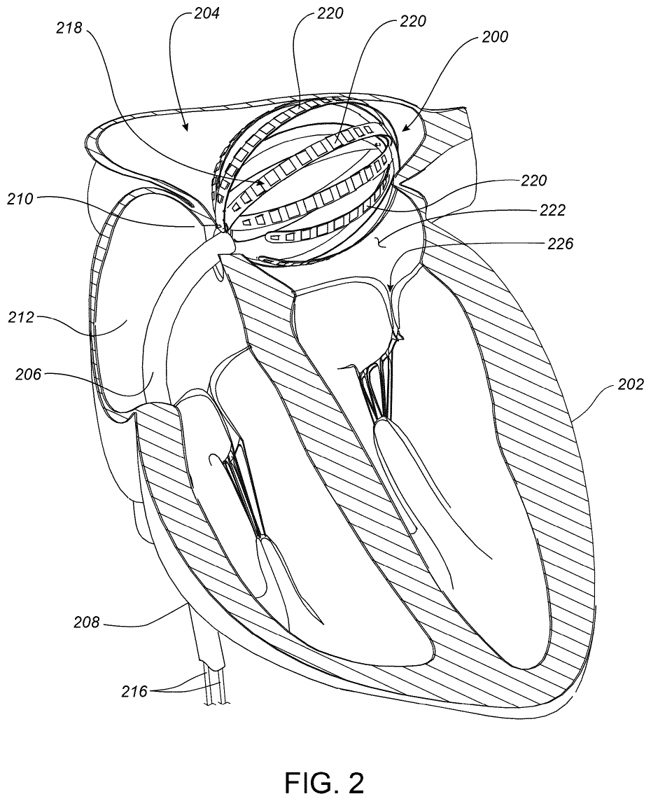

FIGS. 5S-1 and 5S-2 illustrate a graphical representation of at least a portion of a transducer-based device, the graphical representation of the at least the portion of the transducer-based device including transducer graphical elements corresponding to transducers of the transducer-based device, such that the graphical representation presented in FIG. 5S-1 is at least in a state in which a portion of the representation of the transducer-based device is ‘off screen’ and not visible, while another portion of the representation of the transducer-based device is displayed ‘on screen’ at a location away from an encircling path, and the graphical representation presented in FIG. 5S-2 is at least in a state in which at least a larger portion of the transducer-based device representation is displayed ‘on screen’ due to movement of the transducer-based device from the state of FIG. 5S-2, a portion of the representation of the transducer-based device represented over and within the encircling path in the state of FIG. 5S-2, and several of the transducer graphical elements selected over and within the encircling path in the state of FIG. 5S-2, according to some embodiments.

FIGS. 6A, 6B, and 6C include block diagrams of various methods for activating transducers of a transducer-based device, according to some embodiments.

### DETAILED DESCRIPTION

The above-discussed needs in the art are addressed and technical solutions are achieved according to various embodiments of the present invention. In some embodiments, a graphical representation is displayed as including a plurality of graphical elements associated with, or corresponding to, a plurality of transducers that are positionable in a bodily cavity. In some embodiments, user input is received indicating an encircling path. In some embodiments, the path may be formed or displayed among at least a region of a representation of at least a portion of the bodily cavity. In some embodiments, the path may be formed or displayed among at least a first group of at least some of the displayed plurality of graphical elements associated with, or corresponding to, the plurality of transducers. In some embodiments, with the encircling path defined, a region of space may be determined by a data processing device system as being in a particular or determined positional relationship with respect to the encircling path. For example, in some embodiments, the determined positional relationship may be interior or exterior of the encircling path. In some embodiments, in this context, the first region of space may be determined as a region of space that is interior or exterior of the encircling path. In some embodiments, with the region of space determined, a machine-based selection of a transducer set may occur, such that each transducer in the transducer set is selected as having at least part of a corresponding graphical element in the determined region of space. The selected transducer set may then be activated, for example, to cause tissue ablation.

Such an architecture, according to various embodiments, allows, among other possibilities and benefits, a user to easily define an encircling path, and then have the machine or data processing device system automatically select a set of one, more, or all transducers for activation inside or outside of the encircling path. One use case may be for the user to produce an encircling path in a graphical representation around a region corresponding to an anatomical feature of interest, such that some or all transducers that correspond to a physical-space region inside of that path that are located on or near such anatomical feature are machine-selected for performing tissue ablation, thereby allowing efficient selection of multiple transducers by way of an efficient user gesture. In some embodiments, machine-selection of transducers inside, outside, or both, of a user-generated encircling path can efficiently widen a tissue ablation path indicated by the encircling path, to help improve lesion quality, such as an ablation path with improved width, depth, or transmurality. In some embodiments, user-selection of an encircling path may be utilized to set an outer- or inner-extent of a selected pattern of transducers, thereby efficiently allowing a user to cause selection of a group of transducers within or outside of the user-defined outer- or inner-extent, respectively. In some embodiments, an encircling path may be particularly useful at least in the context of utilizing tissue ablation to block unwanted electrical signals through implicated tissue, such as at least in the treatment of atrial fibrillation, since encircling paths may define a closed region to block or trap such unwanted electrical signals.

In the following description, certain specific details are set forth in order to provide a thorough understanding of various embodiments of the invention. However, one skilled in the art will understand that the invention may be practiced at a more general level without these details. In other instances, well-known structures have not been shown or described in detail to avoid unnecessarily obscuring descriptions of various embodiments of the invention.

Any reference throughout this specification to “one embodiment”, “an embodiment”, “an example embodiment”, “an illustrated embodiment”, “a particular embodiment”, and the like means that a particular feature, structure or characteristic described in connection with the embodiment is included in at least one embodiment. Thus, any appearance of the phrase “in one embodiment”, “in an embodiment”, “in an example embodiment”, “in this illustrated embodiment”, “in this particular embodiment”, or the like in this specification is not necessarily always referring to one embodiment or a same embodiment. Furthermore, the particular features, structures or characteristics of different embodiments may be combined in any suitable manner to form one or more other embodiments.

Unless otherwise explicitly noted or required by context, the word “or” is used in this disclosure in a non-exclusive sense. In addition, unless otherwise explicitly noted or required by context, the word “set” is intended to mean one or more. For example, the phrase, “a set of objects” means one or more of the objects. In some embodiments, the word “subset” is intended to mean a set having the same or fewer elements of those present in the subset's parent or superset. In other embodiments, the word “subset” is intended to mean a set having fewer elements of those present in the subset's parent or superset. In this regard, when the word “subset” is used, some embodiments of the present invention utilize the meaning that “subset” has the same or fewer elements of those present in the subset's parent or superset, and other embodiments of the present invention utilize the meaning that “subset” has fewer elements of those present in the subset's parent or superset.

Further, the phrase “at least” is or may be used herein at times merely to emphasize the possibility that other elements may exist besides those explicitly listed. However, unless otherwise explicitly noted (such as by the use of the term “only”) or required by context, non-usage herein of the phrase “at least” nonetheless includes the possibility that other elements may exist besides those explicitly listed. For example, the phrase, ‘based at least on A’ includes A as well as the possibility of one or more other additional elements besides A. In the same manner, the phrase, ‘based on A’ includes A, as well as the possibility of one or more other additional elements besides A. However, the phrase, ‘based only on A’ includes only A. Similarly, the phrase ‘configured at least to A’ includes a configuration to perform A, as well as the possibility of one or more other additional actions besides A. In the same manner, the phrase ‘configured to A’ includes a configuration to perform A, as well as the possibility of one or more other additional actions besides A. However, the phrase, ‘configured only to A’ means a configuration to perform only A.

The word “device”, the word “machine”, the word “system”, and the phrase “device system” all are intended to include one or more physical devices or sub-devices (e.g., pieces of equipment) that interact to perform one or more functions, regardless of whether such devices or sub-devices are located within a same housing or different housings. However, it may be explicitly specified according to various embodiments that a device or machine or device system resides entirely within a same housing to exclude embodiments where the respective device, machine, system, or device system resides across different housings. The word “device” may equivalently be referred to as a “device system” in some embodiments, and the word “system” may equivalently be referred to as a “device system” in some embodiments.

Further, the phrase “in response to” may be used in this disclosure. For example, this phrase may be used in the following context, where an event A occurs in response to the occurrence of an event B. In this regard, such phrase includes, for example, that at least the occurrence of the event B causes or triggers or is a necessary precondition for the event A, according to various embodiments.

The phrase “thermal ablation” as used in this disclosure refers, in some embodiments, to an ablation method in which destruction of tissue occurs by hyperthermia (elevated tissue temperatures) or hypothermia (depressed tissue temperatures). Thermal ablation may include radiofrequency (“RF”) ablation, microwave ablation, or cryo-ablation by way of non-limiting example. RF ablation energy waveforms can take various forms. For example, in some RF ablation embodiments, energy is provided in the form of a continuous waveform. In some RF ablation embodiments, energy is provided in the form of discrete energy applications (e.g., in the form of a duty cycled waveform).

The phrase “pulsed field ablation” (“PFA”) as used in this disclosure refers, in some embodiments, to an ablation method that employs high voltage pulse delivery in a unipolar or bipolar fashion in proximity to target tissue. In some embodiments, each high voltage pulse may be referred to as a discrete energy application. In some embodiments, a grouped plurality of high voltages pulses may be referred to as a discrete energy application. Each high voltage pulse can be a monophasic pulse including a single polarity, or a biphasic pulse including a first component having a first particular polarity and a second component having a second particular polarity opposite the first particular polarity. In some embodiments, the second component of the biphasic pulse follows immediately after the first component of the biphasic pulse. In some embodiments, the first and second components of the biphasic pulse are temporally separated by a relatively small time interval. In some embodiments, each high voltage pulse may include a multiphasic pulse, such as a triphasic pulse, that includes a first component having a first particular polarity, a second component having a second particular polarity opposite the first particular polarity, and a third component having a third particular polarity that is the same as the first particular polarity. The electric field applied by the high voltage pulses in PFA physiologically changes the tissue cells to which the energy is applied (e.g., puncturing or perforating the cell membrane to form various pores therein). If a lower field strength is established, the formed pores may close in time and cause the cells to maintain viability (e.g., a process sometimes referred to as reversible electroporation). If the field strength that is established is greater, then permanent, and sometimes larger, pores form in the tissue cells, the pores allowing loss of control of ion concentration gradients (both inwards and outwards) thereby resulting in cell death (e.g., a process sometimes referred to as irreversible electroporation).

According to some embodiments, the word “fluid” as used in this disclosure should be understood to include any fluid that can be contained within a bodily cavity or can flow into or out of, or both into and out of a bodily cavity via one or more bodily openings positioned in fluid communication with the bodily cavity. In the case of cardiac applications, fluid such as blood will flow into and out of various intracardiac cavities (e.g., a left atrium or a right atrium).

According to some embodiments, the words “bodily opening” as used in this disclosure should be understood to include a naturally occurring bodily opening or channel or lumen; a bodily opening or channel or lumen formed by an instrument or tool using techniques that can include, but are not limited to, mechanical, thermal, electrical, chemical, and exposure or illumination techniques; a bodily opening or channel or lumen formed by trauma to a body; or various combinations of one or more of the above. Various elements having respective openings, lumens, or channels and positioned within the bodily opening (e.g., a catheter sheath) may be present in various embodiments. These elements may provide a passageway through a bodily opening for various devices employed in various embodiments.

The words “bodily cavity” as used in this disclosure should be understood to mean a cavity in a body, in some embodiments. The bodily cavity may be a cavity or chamber provided in a bodily organ (e.g., an intracardiac cavity of a heart).

The word “tissue” as used in some embodiments in this disclosure should be understood to include any surface-forming tissue that is used to form a surface of a body or a surface within a bodily cavity, a surface of an anatomical feature or a surface of a feature associated with a bodily opening positioned in fluid communication with the bodily cavity. The tissue can include part, or all, of a tissue wall or membrane that defines a surface of the bodily cavity. In this regard, the tissue can form an interior surface of the cavity that surrounds a fluid within the cavity. In the case of cardiac applications, tissue can include tissue used to form an interior surface of an intracardiac cavity such as a left atrium or a right atrium. In some embodiments, the word tissue can refer to a tissue having fluidic properties (e.g., blood) and may be referred to as fluidic tissue.

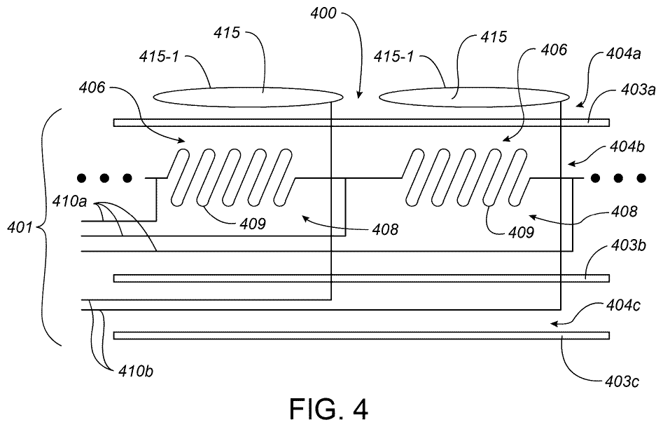

According to some embodiment, the term “transducer” as used in this disclosure should be interpreted broadly as any device capable of transmitting or delivering energy, distinguishing between fluid and tissue, sensing temperature, creating heat, ablating tissue, sensing, sampling or measuring electrical activity of a tissue surface (e.g., sensing, sampling or measuring intracardiac electrograms, or sensing, sampling or measuring intracardiac voltage data), stimulating tissue, providing location information (e.g., in conjunction with a navigation system), or any combination thereof. A transducer may convert input energy of one form into output energy of another form. Without limitation, a transducer may include an electrode that functions as, or as part of, a sensing device included in the transducer, an energy delivery device included in the transducer, or both a sensing device and an energy delivery device included in the transducer. A transducer may be constructed from several parts, which may be discrete components or may be integrally formed. In this regard, although transducers, electrodes, or both transducers and electrodes are referenced with respect to various embodiments, it is understood that other transducers or transducer elements may be employed in other embodiments. It is understood that a reference to a particular transducer in various embodiments may also imply a reference to an electrode, as an electrode may be part of the transducer as shown, e.g., at least with FIG. 4 discussed below.

The term “activation” as used in this disclosure, according to some embodiments, should be interpreted broadly as making active a particular function as related to various transducers disclosed in this disclosure. Particular functions may include, but are not limited to, tissue ablation (e.g., PFA or thermal ablation such as RF), sensing, sampling, or measuring electrophysiological activity (e.g., sensing, sampling, or measuring intracardiac electrogram information, or sensing, sampling, or measuring intracardiac voltage data), sensing, sampling, or measuring temperature and sensing, sampling, or measuring electrical characteristics (e.g., tissue impedance or tissue conductivity). For example, in some embodiments, activation of a tissue ablation function of a particular transducer is initiated by causing energy sufficient for tissue ablation from an energy source device system to be delivered to the particular transducer. Also, in this example, the activation can last for a duration of time concluding when the ablation function is no longer active, such as when energy sufficient for the tissue ablation is no longer provided to the particular transducer. In some contexts and embodiments, however, the word “activation” can merely refer to the initiation of the activating of a particular function, as opposed to referring to both the initiation of the activating of the particular function and the subsequent duration in which the particular function is active. In these contexts, the phrase or a phrase similar to “activation initiation” may be used.

In the following description, some embodiments of the present invention may be implemented at least in part by a data processing device system, or a controller system configured by a software program. Such a program may equivalently be implemented as multiple programs, and some, or all, of such software program(s) may be equivalently constructed in hardware. In this regard, reference to “a program” should be interpreted to include one or more programs.

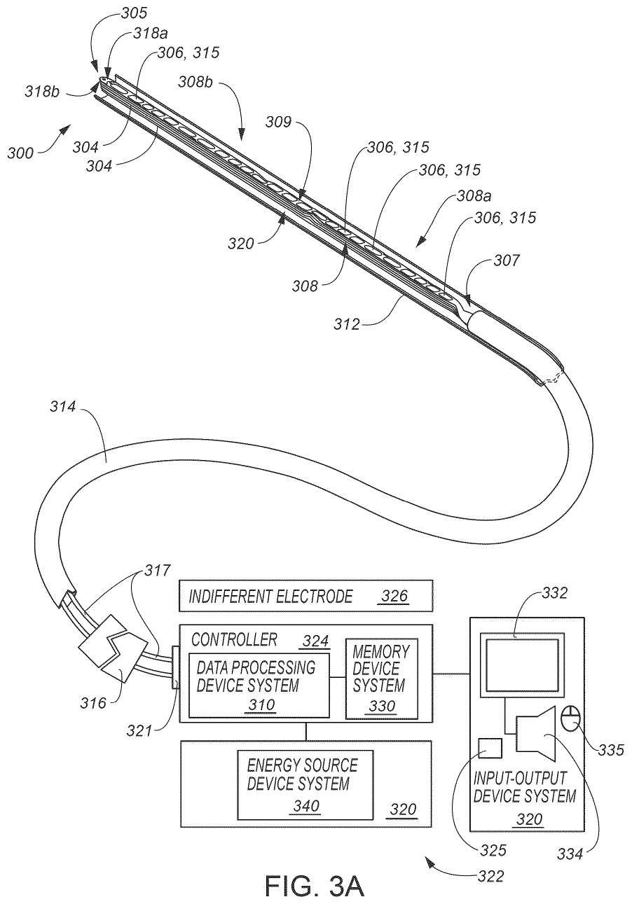

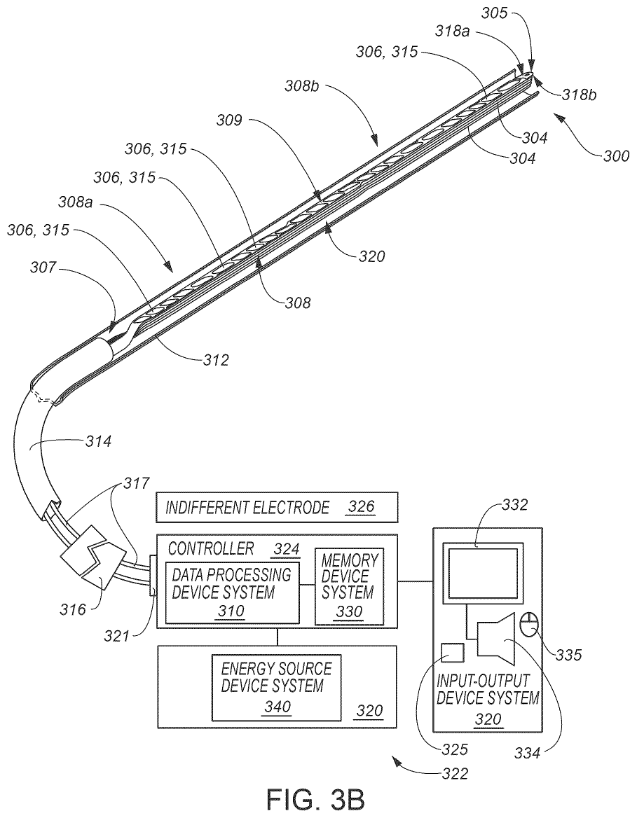

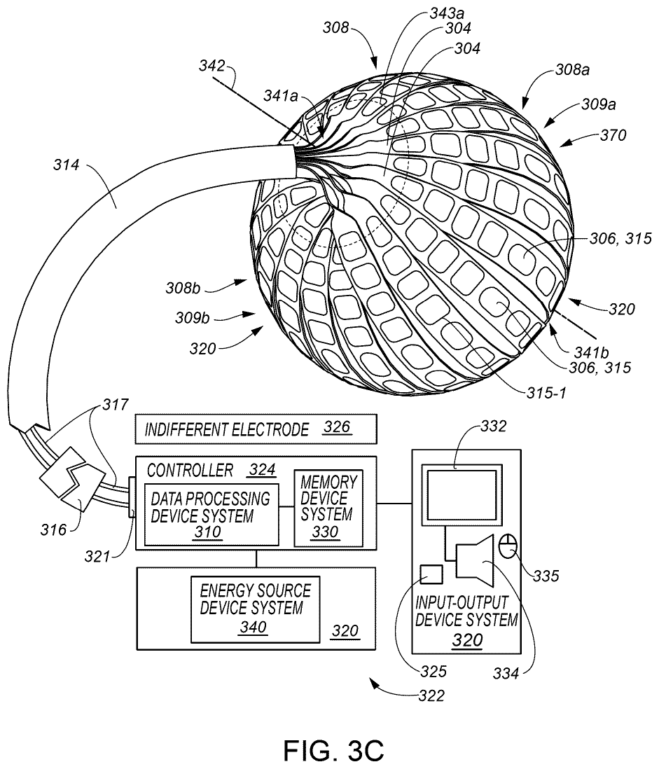

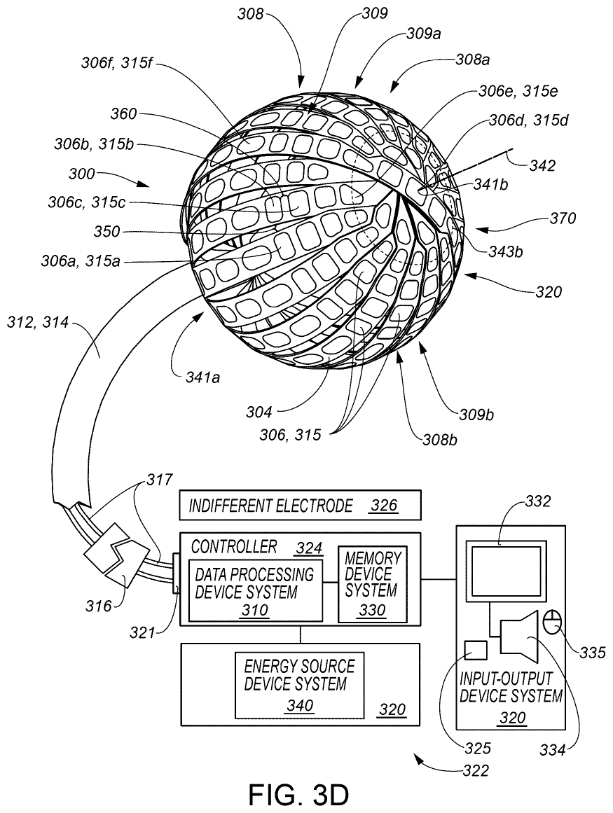

According to some embodiments, the term “program” in this disclosure should be interpreted to include one or more programs including a set of instructions or modules that can be executed by one or more components in a system, such as a controller system or a data processing device system, in order to cause the system to perform one or more operations. The set of instructions or modules may be stored by any kind of memory device, such as those described subsequently with respect to the memory device system 130 or 330 shown in at least FIGS. 1 and 3, respectively. In addition, this disclosure may describe or similarly describe that the instructions or modules of a program are configured to cause the performance of an action. The phrase “configured to” in this context is intended to include, for example, at least (a) instructions or modules that are presently in a form executable by one or more data processing devices to cause performance of the action (e.g., in the case where the instructions or modules are in a compiled and unencrypted form ready for execution), and (b) instructions or modules that are presently in a form not executable by the one or more data processing devices, but could be translated into the form executable by the one or more data processing devices to cause performance of the action (e.g., in the case where the instructions or modules are encrypted in a non-executable manner, but through performance of a decryption process, would be translated into a form ready for execution). Such descriptions should be deemed to be equivalent to describing that the instructions or modules are configured to cause the performance of the action. The word “module” may be defined as a set of instructions. In some instances, this disclosure describes that the instructions or modules of a program perform a function. The word “program” and the word “module” may each be interpreted to include multiple sub-programs or multiple sub-modules, respectively. In this regard, reference to a program or a module may be considered to refer to multiple programs or multiple modules.

Further, it is understood that information or data may be operated upon, manipulated, or converted into different forms as it moves through various devices or workflows. In this regard, unless otherwise explicitly noted or required by context, it is intended that any reference herein to information or data or the like includes modifications to that information or data. For example, “data X” may be encrypted for transmission, and a reference to “data X” is intended to include both its encrypted and unencrypted forms, unless otherwise required or indicated by context. For another example, “image information Y” may undergo a noise filtering process, and a reference to “image information Y” is intended to include both the pre-processed form and the noise-filtered form, unless otherwise required or indicated by context. In other words, both the pre-processed form and the noise-filtered form are considered to be “image information Y”, unless otherwise required or indicated by context. In order to stress this point, the phrase “or a derivative thereof” or the like may be used herein. Continuing the preceding example, the phrase “image information Y or a derivative thereof” refers to both the pre-processed form and the noise-filtered form of “image information Y”, unless otherwise required or indicated by context, with the noise-filtered form potentially being considered a derivative of “image information Y”. However, non-usage of the phrase “or a derivative thereof” or the like nonetheless includes derivatives or modifications of information or data unless otherwise explicitly noted or required by context.

In some embodiments, the phrase “graphical representation” used herein is intended to include a visual representation presented via a display device system and may include computer-generated text, graphics, animations, or one or more combinations thereof, which may include one or more visual representations originally generated, at least in part, by an image-capture device, such as computerized tomography (“CT”) scan images, magnetic resonance imaging (“MRI”) images, or images created from a navigation system (e.g., electric potential navigation system or an electromagnetic navigation system), according to some embodiments. The graphical representation may include various entities depicted in a two-dimensional manner. The graphical representation may include various entities depicted in a three-dimensional manner, in some embodiments.

Example methods are described herein with respect to FIGS. 6A-6C. Such figures include blocks associated with actions, computer-executable instructions, or both, according to various embodiments. It should be noted that the respective instructions associated with any such blocks therein need not be separate instructions and may be combined with other instructions to form a combined instruction set. The same set of instructions may be associated with more than one block. In this regard, the block arrangement shown in each of the method figures herein is not limited to an actual structure of any program or set of instructions or required ordering of method tasks, and such method figures, according to some embodiments, merely illustrate the tasks that instructions are configured to perform, for example, upon execution by a data processing device system in conjunction with interactions with one or more other devices or device systems.

Each of the phrases “derived from” or “derivation of” or “derivation thereof” or the like may be used herein, according to some embodiments, to mean to come from at least some part of a source, be created from at least some part of a source, or be developed as a result of a process in which at least some part of a source forms an input, according to various embodiments. For example, a data set derived from some particular portion of data may include at least some part of the particular portion of data, or may be created from at least part of the particular portion of data, or may be developed in response to a data manipulation process in which at least part of the particular portion of data forms an input. In some embodiments, a data set may be derived from a subset of the particular portion of data. In some embodiments, the particular portion of data is analyzed to identify a particular subset of the particular portion of data, and a data set is derived from the subset. In various ones of these embodiments, the subset may include some, but not all, of the particular portion of data. In some embodiments, changes in at least one part of a particular portion of data may result in changes in a data set derived at least in part from the particular portion of data.

In this regard, each of the phrases “derived from” or “derivation of” or “derivation thereof” or the like may be used herein merely to emphasize the possibility that such data or information may be modified or subject to one or more operations. For example, if a device generates first data for display, the process of converting the generated first data into a format capable of being displayed may alter the first data. This altered form of the first data may be considered a derivative or derivation of the first data. For instance, the first data may be a one-dimensional array of numbers, but the display of the first data may be a color-coded bar chart representing the numbers in the array. For another example, if the above-mentioned first data is transmitted over a network, the process of converting the first data into a format acceptable for network transmission or understanding by a receiving device may alter the first data. As before, this altered form of the first data may be considered a derivative or derivation of the first data. For yet another example, generated first data may undergo a mathematical operation, a scaling, or a combining with other data to generate other data that may be considered derived from the first data. In this regard, it can be seen that data is commonly changing in form or being combined with other data throughout its movement through one or more data processing device systems, and any reference to information or data herein is intended in some embodiments to include these and like changes, regardless of whether or not the phrase “derived from” or “derivation of” or “derivation thereof”′ or the like is used in reference to the information or data. As indicated above, usage of the phrase “derived from” or “derivation of” or “derivation thereof” or the like merely emphasizes the possibility of such changes. Accordingly, in some embodiments, the usage, non-usage, addition of, or deletion of the phrase “derived from” or “derivation of” or “derivation thereof” or the like should have no impact on the interpretation of the respective data or information. For example, the above-discussed color-coded bar chart may be considered a derivative of the respective first data or may be considered the respective first data itself, whether or not the phrase “derived from” or “derivation of” or “derivation thereof” or the like is used, according to some embodiments.

In some embodiments, the term “adjacent”, the term “proximate”, and the like refer at least to a sufficient closeness between the objects or events defined as adjacent, proximate, or the like, to allow the objects or events to interact in a designated way. For example, in the case of physical objects, if object A performs an action on an adjacent or proximate object B, objects A and B would have at least a sufficient closeness to allow object A to perform the action on object B. In this regard, some actions may require contact between the associated objects, such that if object A performs such an action on an adjacent or proximate object B, objects A and B would be in contact, for example, in some instances or embodiments where object A needs to be in contact with object B to successfully perform the action. In some embodiments, the term “adjacent”, the term “proximate”, and the like additionally or alternatively refer to objects or events that do not have another substantially similar object or event between them. For example, object or event A and object or event B could be considered adjacent or proximate (e.g., physically or temporally) if they are immediately next to each other (with no other object or event between them) or are not immediately next to each other but no other object or event that is substantially similar to object or event A, object or event B, or both objects or events A and B, depending on the embodiment, is between them. In the context of graphical elements, discussed at various points in this description, two graphical elements may be considered adjacent, in some embodiments, in which the two graphical elements have no other graphical element between them that is the same in form to any of the two graphical elements. In the context of graphical elements, discussed at various points in this description, two graphical elements may be considered adjacent, in some embodiments, in which the two graphical elements have no other graphical element between them that is the same in function to any of the two graphical elements. In the context of transducer graphical elements, discussed at various points in this description, two transducer graphical elements may be considered adjacent, in some embodiments, in which the two transducer graphical elements have no other transducer graphical element between them. In some embodiments, the term “adjacent”, the term “proximate”, and the like additionally or alternatively refer to at least a sufficient closeness between the objects or events defined as adjacent, proximate, and the like, the sufficient closeness being within a range that does not place any one or more of the objects or events into a different or dissimilar region or time period, or does not change an intended function of any one or more of the objects or events or of an encompassing object or event that includes a set of the objects or events. Different embodiments of the present invention adopt different ones or combinations of the above definitions. Of course, however, the term “adjacent”, the term “proximate”, and the like are not limited to any of the above example definitions, according to some embodiments. In addition, the term “adjacent” and the term “proximate” do not have the same definition, according to some embodiments.

FIG. 1 schematically illustrates a portion of a transducer-activation system or controller system thereof 100 that may be employed to at least select, control, activate, or monitor a function or activation of a number of electrodes or transducers (e.g., ablation transducers configured to cause thermal ablation or ablation transducers configured to cause PFA), according to some embodiments. The system 100 includes a data processing device system 110, an input-output device system 120, and a processor-accessible memory device system 130. The processor-accessible memory device system 130 and the input-output device system 120 are communicatively connected to the data processing device system 110. According to some embodiments, various components such as data processing device system 110, input-output device system 120, and processor-accessible memory device system 130 form at least part of a controller system (e.g., controller system 324 shown in FIG. 3).

The data processing device system 110 includes one or more data processing devices that implement or execute, in conjunction with other devices, such as those in the system 100, various methods and functions described herein, including those described with respect to methods exemplified in FIGS. 6A-6C. Each of the phrases “data processing device”, “data processor”, “processor”, “controller”, “computing device”, “computer” and the like is intended to include any data or information processing device, such as a central processing unit (CPU), a control circuit, a desktop computer, a laptop computer, a mainframe computer, a tablet computer, a personal digital assistant, a cellular or smart phone, and any other device for processing data, managing data, or handling data, whether implemented with electrical, magnetic, optical, quantum, or biological components, or otherwise.

The memory device system 130 includes one or more processor-accessible memory devices configured to store one or more programs and information, including the program(s) and information needed to execute the methods or functions described herein, including those described with respect to FIGS. 6A-6C. The memory device system 130 may be a distributed processor-accessible memory device system including multiple processor-accessible memory devices communicatively connected to the data processing device system 110 via a plurality of computers and/or devices. However, the memory device system 130 need not be a distributed processor-accessible memory system and, consequently, may include one or more processor-accessible memory devices located within a single data processing device or housing.

Each of the phrases “processor-accessible memory” and “processor-accessible memory device” and the like is intended to include any processor-accessible data storage device or medium, whether volatile or nonvolatile, electronic, magnetic, optical, or otherwise, including but not limited to, registers, hard disk drives, Compact Discs, DVDs, flash memories, ROMs, and RAMs. In some embodiments, each of the phrases “processor-accessible memory” and “processor-accessible memory device” is intended to include or be a processor-accessible (or computer-readable) data storage medium. In some embodiments, each of the phrases “processor-accessible memory” and “processor-accessible memory device” may include or may be a non-transitory processor-accessible (or computer-readable) data storage medium. In some embodiments, the processor-accessible memory device system 130 may include or may be a non-transitory processor-accessible (or computer-readable) data storage medium system. In some embodiments, the memory device system 130 may include or may be a non-transitory processor-accessible (or computer-readable) storage medium system or data storage medium system including or consisting of one or more non-transitory processor-accessible (or computer-readable) storage or data storage mediums.

The phrase “communicatively connected” is intended to include any type of connection, whether wired or wireless, between devices, data processors, or programs between which data may be communicated. Further, the phrase “communicatively connected” is intended to include a connection between devices or programs within a single data processor or computer, a connection between devices or programs located in different data processors or computers, and a connection between devices not located in data processors or computers at all. In this regard, although the memory device system 130 is shown separately from the data processing device system 110 and the input-output device system 120, one skilled in the art will appreciate that the memory device system 130 may be located completely or partially within the data processing device system 110 or the input-output device system 120. Further in this regard, although the input-output device system 120 is shown separately from the data processing device system 110 and the memory device system 130, one skilled in the art will appreciate that such system may be located completely or partially within the data processing system 110 or the memory device system 130, for example, depending upon the contents of the input-output device system 120. Further still, the data processing device system 110, the input-output device system 120, and the memory device system 130 may be located entirely within the same device or housing or may be separately located, but communicatively connected, among different devices or housings. In the case where the data processing device system 110, the input-output device system 120, and the memory device system 130 are located within the same device, the system 100 of FIG. 1 can be implemented by a single application-specific integrated circuit (ASIC) in some embodiments.

The input-output device system 120 may include a mouse, a keyboard, a touch screen, another computer, or any device or combination of devices from which a desired selection, desired information, instructions, or any other data is input to the data processing device system 110. The input-output device system 120 may include a user-activatable control system that is responsive to a user action. The user-activatable control system may include at least one control element that may be activated or deactivated on the basis of a particular user action. The input-output device system 120 may include any suitable interface for receiving information, instructions or any data from other devices and systems described in various ones of the embodiments. In this regard, the input-output device system 120 may include various ones of other systems described in various embodiments. For example, the input-output device system 120 may include at least a portion of a transducer-based device. The phrase “transducer-based device” or “transducer-based device system” is intended to include one or more physical systems that include various transducers. A PFA device system that includes one or more transducers may be considered a transducer-based device or device system, according to some embodiments.

The input-output device system 120 also may include an image generating device system, a display device system, a speaker or audio output device system, a computer, a processor-accessible memory device system, a network-interface card or network-interface circuitry, or any device or combination of devices to which information, instructions, or any other data is output by the data processing device system 110. In this regard, the input-output device system 120 may include various other devices or systems described in various embodiments. The input-output device system 120 may include any suitable interface for outputting information, instructions, or data to other devices and systems described in various ones of the embodiments. If the input-output device system 120 includes a processor-accessible memory device, such memory device may, or may not, form part, or all, of the memory device system 130. The input-output device system 120 may include any suitable interface for outputting information, instructions, or data to other devices and systems described in various ones of the embodiments. In some embodiments, the input-output device system 120 may include a transducer-based device, as discussed above, and in some embodiments, the transducer-based device may act as a device or device system that provides information to, receives instructions or energy from, or both provides information to and receives instructions or energy from the data processing device system 110. In this regard, the input-output device system 120 may include various devices or systems described in various embodiments.

Various embodiments of transducer-based devices are described herein in this disclosure. Some of the described devices are PFA devices that are percutaneously or intravascularly deployed. Some of the described devices are movable between a delivery or unexpanded configuration (e.g., FIGS. 3A and 3B discussed below) in which a portion of the device is sized for passage through a bodily opening leading to a bodily cavity, and an expanded or deployed configuration (e.g., FIGS. 2, 3C, and 3D discussed below) in which the portion of the device has a size too large for passage through the bodily opening leading to the bodily cavity. An example of an expanded or deployed configuration, in some embodiments, is when the portion of the transducer-based device is in its intended-deployed-operational state, which may be inside the bodily cavity when, e.g., performing an intended therapeutic or diagnostic procedure for a patient, or which may be outside the bodily cavity when, e.g., performing testing, quality control, or other evaluation of the device. Another example of the expanded or deployed configuration, in some embodiments, is when the portion of the transducer-based device is being changed from the delivery configuration to the intended-deployed-operational state to a point where the portion of the device now has a size too large for passage through the bodily opening leading to the bodily cavity.

In some example embodiments, the device includes transducers that sense characteristics (e.g., convective cooling, permittivity, force) that distinguish between fluid, such as a fluidic tissue (e.g., blood), and tissue forming an interior surface of the bodily cavity. Such sensed characteristics can allow a medical system to map the cavity, for example, using positions of openings or ports into and out of the cavity to determine a position or orientation (e.g., pose), or both, of the portion of the device in the bodily cavity. In some example embodiments, the described systems employ a navigation system or electro-anatomical mapping system including electromagnetic-based systems and electropotential-based systems to determine a positioning of a portion of a device in a bodily cavity. In some example embodiments, the described devices are part of a transducer-activation system capable of ablating tissue in a desired pattern within the bodily cavity using various techniques (e.g., via thermal ablation, PFA, etc., according to various embodiments).

In some example embodiments, the devices are capable of sensing various cardiac functions (e.g., electrophysiological activity including intracardiac voltages). In some example embodiments, the devices are capable of providing stimulation (e.g., electrical stimulation) to tissue within the bodily cavity. Electrical stimulation may include pacing.

FIG. 2 is a representation of a transducer-based device 200 useful in investigating or treating a bodily organ, for example, a heart 202, according to at least one example embodiment.

Transducer-based device 200 can be percutaneously or intravascularly inserted into a portion of the heart 202, such as an intra-cardiac cavity like left atrium 204. In this example, the transducer-based device 200 is part of a catheter 206 inserted via the inferior vena cava 208 and penetrating through a bodily opening in transatrial septum 210 from right atrium 212. (In this regard, transducer-based devices or device systems described herein that include a catheter may also be referred to as catheter devices or catheter-based devices, in some embodiments). In other embodiments, other paths may be taken.

Catheter 206 includes an elongated flexible rod or shaft member appropriately sized to be delivered percutaneously or intravascularly. Various portions of catheter 206 may be steerable. Catheter 206 may include one or more lumens. The lumen(s) may carry one or more communications or power paths, or both. For example, the lumens(s) may carry one or more electrical conductors 216 (two shown). Electrical conductors 216 provide electrical connections to transducer-based device 200 that are accessible externally from a patient in which the transducer-based device 200 is inserted.

Transducer-based device 200 includes a frame or structure 218 which assumes an unexpanded configuration for delivery to left atrium 204. Structure 218 is expanded (e.g., shown in a deployed or expanded configuration in FIG. 2) upon delivery to left atrium 204 to position a plurality of transducers 220 (three called out in FIG. 2) proximate the interior surface formed by tissue 222 of left atrium 204. In some embodiments, at least some of the transducers 220 are used to sense a physical characteristic of a fluid (e.g., blood) or tissue 222, or both, that may be used to determine a position or orientation (e.g., pose), or both, of a portion of a device 200 within, or with respect to left atrium 204. For example, transducers 220 may be used to determine a location of pulmonary vein ostia or a mitral valve 226, or both. In some embodiments, at least some of the transducers 220 may be used to selectively ablate portions of the tissue 222. For example, some of the transducers 220 may be used to ablate a pattern around the bodily openings, ports or pulmonary vein ostia, for instance to reduce or eliminate the occurrence of atrial fibrillation. In some embodiments, at least some of the transducers 220 are used to ablate cardiac tissue. In some embodiments, at least some of the transducers 220 are used to sense or sample intra-cardiac voltage data or sense or sample intra-cardiac electrogram data. In some embodiments, at least some of the transducers 220 are used to sense or sample intra-cardiac voltage data or sense or sample intra-cardiac electrogram data while at least some of the transducers 220 are concurrently ablating cardiac tissue. In some embodiments, at least one of the sensing or sampling transducers 220 is provided by at least one of the ablating transducers 220. In some embodiments, at least a first one of the transducers 220 senses or samples intra-cardiac voltage data or intra-cardiac electrogram data at a location at least proximate to a tissue location ablated by at least a second one of the transducers 220. In some embodiments, the first one of the transducers 220 is other than the second one of the transducers 220.

FIGS. 3A, 3B, 3C, and 3D (collectively, FIG. 3) include a transducer-based device system (e.g., a portion thereof shown schematically) that includes a transducer-based device 300 according to some embodiments. Transducer-based device 300 includes a plurality of elongate members 304 (not all of the elongate members called out in each of FIGS. 3A, 3B, 3C and 3D) and a plurality of transducers 306 (not all of the transducers called out in FIG. 3) (some of the transducers 306 called out in FIG. 3D as 306a, 306b, 306c, 306d, 306e and 306f). FIG. 3B includes a representation of a portion of the transducer-based device 300 shown in FIG. 3A, but as viewed from a different viewing direction. FIG. 3D includes a representation of a portion of the transducer-based device 300 shown in FIG. 3C, but as viewed from a different viewing direction. It is noted that for clarity of illustration, all the elongate members shown in FIGS. 3C and 3D are not represented in FIGS. 3A and 3B. The plurality of transducers 306 are positionable within a bodily cavity. For example, in some embodiments, the transducers 306 are able to be positioned in a bodily cavity by movement into, within, or into and within the bodily cavity, with or without a change in a configuration of the plurality of transducers 306. In some embodiments, the plurality of transducers 306 are arranged to form a two- or three-dimensional distribution, grid or array of the transducers capable of mapping, ablating or stimulating an inside surface of a bodily cavity or lumen without requiring mechanical scanning. As shown, for example, in FIGS. 3A and 3B, the plurality of transducers 306 are arranged in a distribution receivable in a bodily cavity. In various ones of the FIGS. 3, each of at least some of transducers 306 includes a respective electrode 315 (not all of the electrodes 315 called out in each of the FIGS. 3, some of the electrodes in FIG. 3D called out as 315a, 315b, 315c, 315d, 315e and 315f).

According to some embodiments, the elongate members 304 may be arranged in a frame or structure 308 that is selectively movable between an unexpanded or delivery configuration (e.g., as shown in FIGS. 3A, 3B) and an expanded or deployed configuration (e.g., as shown in FIGS. 3C, 3D) that may be used during a positioning of the elongate members 304 against a tissue surface within the bodily cavity or during a positioning of the elongate members 304 in the vicinity of the tissue surface. At least the expanded or deployed configuration shown in FIGS. 3C and 3D is an example of a three-dimensional distribution of the transducers 306. In some embodiments, structure 308 has a size in the unexpanded or delivery configuration suitable for delivery through a bodily opening (e.g., via catheter sheath 312 (shown in FIGS. 3A and 3B but removed from FIGS. 3C and 3D for clarity)) to the bodily cavity. At least in a state in which the structure 308 is in the expanded or deployed configuration, the structure 308 may be considered to have two opposing poles 341a and 341b, marked by the intersection with axis 342 extending through the structure 308 as shown in FIGS. 3C and 3D. At least some of the plurality of transducers 306 are circumferentially arranged, e.g., in successive ring-like arrangements, about each of the poles 341a and 341b according to some embodiments. Two such ring-like arrangements are illustrated, for example, as broken-line rings 343a and 343b in FIG. 3C and FIG. 3D, respectively. At least some of the plurality of transducers 306 are arranged in a plurality of groups of the transducers 306, the groups of transducers 306 arranged like lines of longitude (e.g., along respective elongate members 304) about the structure 308 between each of the poles 341a and 341b, according to some embodiments. At least some of the plurality of transducers 306 are arranged in a plurality of groups of the transducers 306, the transducers in each group of transducers 306 arrayed along a path (e.g., along at least a respective portion of a respective elongate member 304) that extends toward the pole 341a, the pole 341b, or both poles 341a and 341b, according to some embodiments. In some embodiments, each path extends like a line of longitude between the poles 341a and 341b.

In various embodiments, catheter sheath 312 typically includes a length sufficient to allow the catheter sheath to extend between a location at least proximate a bodily cavity into which the structure 308 is to be delivered and a location outside a body including the bodily cavity. In some embodiments, structure 308 has a size in the expanded or deployed configuration too large for delivery through a bodily opening (e.g., via catheter sheath 312) to the bodily cavity. The elongate members 304 may form part of a flexible circuit structure (e.g., also known as a flexible printed circuit board (PCB) circuit). The elongate members 304 may include a plurality of different material layers. Each of the elongate members 304 may include a plurality of different material layers. The structure 308 may include a shape memory material, for instance, Nitinol. The structure 308 can include a metallic material, for instance stainless steel, or non-metallic material, for instance polyimide, or both a metallic and non-metallic material by way of non-limiting example. The incorporation of a specific material into structure 308 may be motivated by various factors including the specific requirements of each of the unexpanded or delivery configuration and expanded or deployed configuration, the required position or orientation (e.g., pose), or both of structure 308 in the bodily cavity or the requirements for successful ablation of a desired pattern.

FIG. 4 is a schematic side elevation view of at least a portion of a transducer-based device 400 that includes a flexible circuit structure 401 that is employed to provide a plurality of transducers 406 (two called out) according to an example embodiment. In some embodiments, the flexible circuit structure 401 may form part of a structure (e.g., structure 308) that is selectively movable between a delivery configuration sized for percutaneous delivery and expanded or deployed configurations sized too large for percutaneous delivery. In some embodiments, the flexible circuit structure 401 may be located on, or form at least part of, a structural component (e.g., elongate member 304) of a transducer-based device system.

The flexible circuit structure 401 can be formed by various techniques including flexible printed circuit techniques. In some embodiments, the flexible circuit structure 401 includes various layers including flexible layers 403a, 403b and 403c (e.g., collectively flexible layers 403). In some embodiments, each of flexible layers 403 includes an electrical insulator material (e.g., polyimide). One or more of the flexible layers 403 can include a different material than another of the flexible layers 403. In some embodiments, the flexible circuit structure 401 includes various electrically conductive layers 404a, 404b and 404c (collectively electrically conductive layers 404) that are interleaved with the flexible layers 403. In some embodiments, each of the electrically conductive layers 404 is patterned to form various electrically conductive elements. For example, electrically conductive layer 404a is patterned to form a respective electrode 415 of each of the transducers 406. Electrodes 415 have respective electrode edges 415-1 that form a periphery of an electrically conductive surface associated with the respective electrode 415. It is noted that other electrodes employed in other embodiments may have electrode edges arranged to form different electrodes shapes (for example, as shown by electrode edges 315-1 in FIG. 3C).

Electrically conductive layer 404b is patterned, in some embodiments, to form respective temperature sensors 408 for each of the transducers 406 as well as various leads 410a arranged to provide electrical energy to the temperature sensors 408. In some embodiments, each temperature sensor 408 includes a patterned resistive member 409 (two called out) having a predetermined electrical resistance. In some embodiments, each resistive member 409 includes a metal having relatively high electrical conductivity characteristics (e.g., copper). In some embodiments, electrically conductive layer 404c is patterned to provide portions of various leads 410b arranged to provide an electrical communication path to electrodes 415. In some embodiments, leads 410b are arranged to pass though vias in flexible layers 403a and 403b to connect with electrodes 415. Although FIG. 4 shows flexible layer 403c as being a bottom-most layer, some embodiments may include one or more additional layers underneath flexible layer 403c, such as one or more structural layers, such as a steel or composite layer. These one or more structural layers, in some embodiments, are part of the flexible circuit structure 401 and can be part of, e.g., elongate member 304. In some embodiments, the one or more structural layers may include at least one electrically conductive surface (e.g., a metallic surface) exposed to blood flow. In addition, although FIG. 4 shows only three flexible layers 403a-403c and only three electrically conductive layers 404a-404c, it should be noted that other numbers of flexible layers, other numbers of electrically conductive layers, or both, can be included.

In some embodiments, electrodes 415 are employed to selectively deliver ablation energy (e.g., thermal ablation energy or PFA energy) to various tissue structures within a bodily cavity (e.g., an intra-cardiac cavity or chamber). The energy delivered to the tissue structures may be sufficient for ablating portions of the tissue structures. The energy delivered to the tissue may be delivered to cause monopolar tissue ablation, bipolar tissue ablation or blended monopolar-bipolar tissue ablation by way of non-limiting example.

Energy that is sufficient for tissue ablation may be dependent upon factors including transducer location, size, shape, relationship with respect to another transducer or a bodily cavity, material or lack thereof between transducers, et cetera. Typically, a larger electrode (e.g., an electrode with a relatively large surface area) will achieve a given ablation depth sooner than a smaller electrode. Put differently, a maximum ablation depth of a relatively smaller electrode is typically shallower than that of a relatively larger electrode when ablating under the same control parameters as a relatively larger electrode.

In some embodiments, each electrode 415 is employed to sense or sample an electrical potential in the tissue proximate the electrode 415 at a same or different time than delivering energy sufficient for tissue ablation. In some embodiments, each electrode 415 is employed to sense or sample intra-cardiac voltage data in the tissue proximate the electrode 415. In some embodiments, each electrode 415 is employed to sense or sample data in the tissue proximate the electrode 415 from which an electrogram (e.g., an intra-cardiac electrogram) may be derived. In some embodiments, each resistive member 409 is positioned adjacent a respective one of the electrodes 415. In some embodiments, each of the resistive members 409 is positioned in a stacked or layered array with a respective one of the electrodes 415 to form a respective one of the transducers 406. In some embodiments, the resistive members 409 are connected in series to allow electrical current to pass through all of the resistive members 409. In some embodiments, leads 410a are arranged to allow for a sampling of electrical voltage in between each resistive members 409. This arrangement allows for the electrical resistance of each resistive member 409 to be accurately measured. The ability to accurately measure the electrical resistance of each resistive member 409 may be motivated by various reasons including determining temperature values at locations at least proximate the resistive member 409 based at least on changes in the resistance caused by convective cooling effects (e.g., as provided by blood flow).

Referring to FIGS. 3A, 3B, 3C, and 3D transducer-based device 300 can communicate with, receive power from or be controlled by a transducer-activation system 322. In some embodiments, the transducer-activation system 322 represents one or more particular implementations of the system 100 illustrated in FIG. 1. In some embodiments, the transducer-based device 300 or the transducer-based device 200 may be considered part of the transducer-activation system 322 or 100. However, the transducer activation system 322 or 100, according to various embodiments, is not limited to including or interacting with either of the particular transducer-based devices 200, 300, and may include or interact with other one or more other types of transducer-based devices in some embodiments.

In some embodiments, elongate members 304 can form a portion of an elongated cable 316 of leads 317 (e.g., control leads, data leads, power leads or any combination thereof), for example, by stacking multiple layers, and terminating at a connector 321 or other interface with transducer-activation system 322. The leads 317 may correspond to the electrical connectors 216 in FIG. 2 in some embodiments. The transducer-activation device system 322 may include a controller 324 that includes a data processing device system 310 (e.g., which may be a particular implementation of data processing device system 110 from FIG. 1) and a memory device system 330 (e.g., which may be a particular implementation of the memory device system 130 from FIG. 1) that stores data and instructions that are executable by the data processing device system 310 to process information received from transducer-based device 300 or to control operation of transducer-based device 300, for example, activating various selected transducers 306 to ablate tissue and control a user interface (e.g., of input-output device system 320) according to various embodiments including at least those described below with respect to various ones of FIG. 5. Controller 324 may include one or more controllers.

Transducer-activation device system 322 includes an input-output device system 320 (e.g., which may be a particular implementation of the input-output device system 120 from FIG. 1) communicatively connected to the data processing device system 310 (e.g., via controller 324 in some embodiments). Input-output device system 320 may include a user-activatable control that is responsive to a user action. Input-output device system 320 may include one or more user interfaces or input/output (I/O) devices, for example, one or more display device systems 332, speaker device systems 334, one or more keyboards, one or more mice (e.g., mouse 335), one or more joysticks, one or more track pads, one or more touch screens or other transducers to transfer information to, from, or both to and from a user, for example, a care provider such as a physician or technician. For example, output from a mapping process may be displayed on a display device system 332. Input-output device system 320 may include one or more user interfaces or input/output (I/O) devices, for example, one or more display device systems 332, speaker device systems 334, keyboards, mice, joysticks, track pads, touch screens or other transducers employed by a user to indicate a particular selection or series of selections of various graphical information. Input-output device system 320 may include a sensing device system 325 configured to detect various characteristics including, but not limited to, at least one of tissue characteristics (e.g., electrical characteristics such as tissue impedance, tissue conductivity, tissue type, tissue thickness) and thermal characteristics such as temperature. In this regard, the sensing device system 325 may include one, some, or all of the transducers 306 (or 406 of FIG. 4) of the transducer-based device 300, including the internal components of such transducers shown in FIG. 4, such as the electrodes 415 and temperature sensors 408.

Transducer-activation device system 322 may also include an energy source device system 340 including one or more energy source devices connected to transducers 306. In this regard, although various ones of FIG. 3 show a communicative connection between the energy source device system 340 and the controller 324 (and its data processing device system 310), the energy source device system 340 may also be connected to the transducers 306 via a communicative connection that is independent of the communicative connection with the controller 324 (and its data processing device system 310). For example, the energy source device system 340 may receive control signals via the communicative connection with the controller 324 (and its data processing device system 310), and, in response to such control signals, deliver energy to, receive energy from, or both deliver energy to and receive energy from one or more of the transducers 306 via a communicative connection with such transducers 306 (e.g., via one or more communication lines through catheter body or shaft 314, elongated cable 316 or catheter sheath 312) that does not pass through the controller 324. In this regard, the energy source device system 340 may provide results of its delivering energy to, receiving energy from, or both delivering energy to and receiving energy from one or more of the transducers 306 to the controller 324 (and its data processing device system 310) via the communicative connection between the energy source device system 340 and the controller 324.

The energy source device system 340 may, for example, be connected to various selected transducers 306 to selectively provide energy in the form of electrical current or power (e.g., thermal ablation energy or PFA energy), light or low temperature fluid to the various selected transducers 306 to cause ablation of tissue. The energy source device system 340 may, for example, selectively provide energy in the form of electrical current to various selected transducers 306 and measure a temperature characteristic, an electrical characteristic, or both at a respective location at least proximate each of the various transducers 306. The energy source device system 340 may include various electrical current sources or electrical power sources as energy source devices. In some embodiments, an indifferent electrode 326 is provided to receive at least a portion of the energy transmitted by at least some of the transducers 306. Consequently, although not shown in various ones of FIGS. 3, the indifferent electrode 326 may be communicatively connected to the energy source device system 340 via one or more communication lines in some embodiments. In addition, although shown separately in various ones of FIGS. 3, indifferent electrode 326 may be considered part of the energy source device system 340 in some embodiments. In various embodiments, indifferent electrode 326 is positioned on an external surface (e.g., a skin-based surface) of a body that comprises the bodily cavity into which at least transducers 306 are to be delivered.

It is understood that input-output device system 320 may include other systems. In some embodiments, input-output device system 320 may optionally include energy source device system 340, transducer-based device 300 or both energy source device system 340 and transducer-based device 300 by way of non-limiting example. Input-output device system 320 may include the memory device system 330 in some embodiments.

Structure 308 can be delivered and retrieved via a catheter member, for example, a catheter sheath 312. In some embodiments, a structure provides expansion and contraction capabilities for a portion of the medical device (e.g., an arrangement, distribution or array of transducers 306). The transducers 306 can form part of, be positioned or located on, mounted, or otherwise carried on the structure and the structure may be configurable to be appropriately sized to slide within catheter sheath 312 in order to be deployed percutaneously or intravascularly. FIGS. 3A, 3B show one embodiment of such a structure. In some embodiments, each of the elongate members 304 includes a respective distal end 305 (only one called out in each of FIGS. 3A, 3B), a respective proximal end 307 (only one called out in each of FIGS. 3A, 3B) and an intermediate portion 309 (only one called out in each of FIGS. 3A, 3B) positioned between the proximal end 307 and the distal end 305. The respective intermediate portion 309 of each elongate member 304 includes a first or front surface 318a that is positionable to face an interior tissue surface within a bodily cavity and a second or back surface 318b opposite across a thickness of the intermediate portion 309 from the front surface 318a. In some embodiments, each of the elongate members 304 is arranged front surface 318a-toward-back surface 318b in a stacked array during an unexpanded or delivery configuration similar to that described in International Publication No. WO 2012/100184, published Jul. 26, 2012 (Fernando Lopes et al.) and International Publication No. WO 2012/100185, published Jul. 26, 2012 (Fernando Lopes et al.). In many cases a stacked array allows the structure 308 to have a suitable size for percutaneous or intravascular delivery. In some embodiments, the elongate members 304 are arranged to be introduced into a bodily cavity distal end 305 first. A flexible, elongated, catheter body 314 is used to deliver structure 308 through catheter sheath 312 according to some embodiments.

In a manner similar to that described in International Publication No. WO 2012/100184, published Jul. 26, 2012 (Fernando Lopes et al.) and International Publication No. WO 2012/100185, published Jul. 26, 2012 (Fernando Lopes et al.), each of the elongate members 304 is arranged in a fanned arrangement 370 in FIGS. 3C, 3D. In some embodiments, the fanned arrangement 370 is formed during the expanded or deployed configuration in which structure 308 is manipulated to have a size too large for percutaneous or intravascular delivery. In some embodiments, structure 308 includes a proximal portion 308a having a first domed shape 309a and a distal portion 308b having a second domed shape 309b. In some embodiments, the proximal and the distal portions 308a, 308b each include respective portions of elongate members 304. In some embodiments, the structure 308 is arranged to be delivered distal portion 308b first into a bodily cavity when the structure is in the unexpanded or delivery configuration as shown in FIGS. 3A, 3B. In various embodiments, the proximal and distal portions 308a, 308b do not include a domed shape in the delivery configuration (for example, as shown in FIGS. 3A, 3B). In some embodiments, the first domed shape 309a of the proximal portion 308a and the second domed shape 309b of the distal portion 308b are arranged in a clam shell configuration in the expanded or deployed configuration shown in FIGS. 3C, 3D.

The transducers 306 can be arranged in various distributions or arrangements in various embodiments. In some embodiments, various ones of the transducers 306 are spaced apart from one another in a spaced apart distribution in the delivery configuration shown in FIGS. 3A, 3B. In some embodiments, various ones of the transducers 306 are arranged in a spaced apart distribution in the deployed configuration shown in FIGS. 3C, 3D. In some embodiments, various pairs of transducers 306 are spaced apart with respect to one another. In some embodiments, various spaces are located between various pairs of the transducers 306. For example, in FIG. 3D the transducer-based device 300 includes at least a first transducer 306a, a second transducer 306b and a third transducer 306c (all collectively referred to as transducers 306). In some embodiments each of the first, the second and the third transducers 306a, 306b and 306c are adjacent transducers in the spaced apart distribution (e.g., first transducer 306a is adjacent second transducer 306b, second transducer 306b is adjacent third transducer 306c, and first transducer 306a is adjacent third transducer 306c). In some embodiments, the first and the second transducers 306a, 306b are located on different elongate members 304 while the second and the third transducers 306b, 306c are located on a same elongate member 304. In some embodiments, a first space 350 is between the first and the second transducers 306a, 306b. In various embodiments, a first space 350 is between the respective electrodes 315a, 315b of the first and the second transducers 306a, 306b. In some embodiments, the first space 350 is not associated with any physical portion of structure 308. In some embodiments, a second space 360 associated with a physical portion of device 300 (e.g., a portion of an elongate member 304) is between the second and the third transducers 306b, 306c. In various embodiments, the second space 360 is between the respective electrodes 315b, 315c of the second and the third transducers 306b, 306c. In some embodiments, each of the first and the second spaces 350, 360 does not include a transducer of transducer-based device 300. In some embodiments, each of the first and the second spaces 350, 360 does not include any transducer. It is noted that other embodiments need not employ a group of elongate members 304 as employed in the illustrated embodiment. For example, other embodiments may employ a structure having one or more surfaces, at least a portion of the one or more surfaces defining one or more openings in the structure. In these embodiments, a space not associated with any physical portion of the structure may extend over at least part of an opening of the one or more openings.

In other example embodiments, other structures besides those shown in FIGS. 2 and 3A-3D may be employed to support or carry transducers of a transducer-based device such as a transducer-based catheter. For example, an elongated catheter member may be used to distribute the transducers in a linear or curvilinear array. Basket catheters or balloon catheters may be used to distribute the transducers in a two-dimensional or three-dimensional array.

FIGS. 6A-6C include respective data generation and flow diagrams, which may implement various embodiments of methods 600 (split across FIGS. 6A and 6B, with FIG. 6C showing additional embodiments) by way of associated computer-executable instructions according to some example embodiments. In various example embodiments, a memory device system (e.g., memory device systems 130, 330) is communicatively connected to a data processing device system (e.g., data processing device systems 110 or 310, otherwise stated herein as “e.g., 110, 310”) and stores a program executable by the data processing device system to cause the data processing device system to execute various embodiments of methods 600 via interaction with at least, for example, a transducer-based device (e.g., transducer-based devices 200, 300, or 400, in some embodiments). In these various embodiments, the program may include instructions configured to perform, or cause to be performed, various ones of the instructions associated with execution of various embodiments of methods 600. In some embodiments, the methods 600 may include a subset of the associated blocks or additional blocks than those shown in FIGS. 6A-6C. In some embodiments, the methods 600 may include a different sequence indicated between various ones of the associated blocks shown in FIGS. 6A-6C. In FIGS. 6A-6C, some blocks are illustrated in broken line, such as, for example, blocks 602a and 602b in FIG. 6A. Such broken line blocks illustrate possible implementation details for the block in which the broken line block resides, according to some embodiments. For example, broken line blocks 602a and 602b illustrate possible implementation details for block 602, according to some embodiments. In this regard, for example, some embodiments of block 602 may adopt the implementation details of block 602a, some embodiments of block 602 may adopt the implementation details of block 602b, some embodiments of block 602 may adopt the implementation details of block 602a and block 602b, and some embodiments of block 602 may adopt implementation details other than those of block 602a and block 602b. The features recited by any block are not intended to be exclusive, and the adopting of any recited features of any particular block in a particular embodiment does not prevent the inclusion of any other features, according to some embodiments, unless the features cannot work together (i.e., are mutually exclusive).

According to some embodiments, methods 600 may include block 602 associated with computer-executable instructions (e.g., graphical representation instructions (which also may be referred to at least as graphical interface instructions or display instructions) provided by a program) configured to cause an input-output device system (e.g., 120, 320) to display a graphical representation. In some embodiments associated with block 602a, the graphical representation includes a graphical representation of a transducer-based device (e.g., 200, 300, or 400 in some embodiments). For example, at least FIG. 5G, discussed in more detail below, illustrates a graphical representation 543 of a transducer-based device, similar to transducer-based device 300. In some embodiments, the graphical representation includes a plurality of graphical elements associated with or corresponding to a plurality of transducers positionable in a bodily cavity. For example, at least FIG. 5G, discussed in more detail below, illustrates a graphical representation 500 including graphical elements 502 (two called out in FIG. 5G) associated with a plurality of transducers of the illustrated transducer-based device. In some embodiments, the graphical representation (e.g., graphical representation 500) includes a plurality of graphical elements (e.g., graphical elements 502) corresponding to a plurality of transducers positionable in a bodily cavity. In some embodiments, each of the plurality of graphical elements may correspond to a respective at least one of the plurality of transducers. According to various embodiments, the transducers of the plurality of transducers may be individually selectable. According to various embodiments, the transducers of the plurality of transducers may be individually addressable. According to various embodiments, the transducers of the plurality of transducers may be individually activatable. In some embodiments associated with block 602b in FIG. 6A, the graphical representation includes a representation of at least a portion of a bodily cavity. For example, at least FIG. 5G, discussed in more detail below, illustrates a graphical representation 542 of at least a portion of a bodily cavity, which is an atrium in the case of FIG. 5G.

FIG. 5A illustrates a graphical interface including a graphical representation 500 provided by the input-output device system according to some embodiments provided in accordance with graphical representation instructions associated with block 602 or block 602a in FIG. 6A. In some embodiments, the graphical representation 500 includes a three-dimensional graphical representation of at least a portion of a transducer-based device (e.g., like structure 308 in at least FIG. 3D). However, inclusion of the transducer-based device is omitted in some embodiments associated with block 602a, in which, for example, graphical elements associated with a plurality of transducers may be displayed without a graphical representation of a larger transducer-based device.

The instructions associated with block 602 or block 602a may be configured to access a predefined model (e.g., a computer-aided-design (“CAD”) or other computer-readable model stored in memory device system 130, 330) of the at least the portion of the transducer-based device (e.g., the plurality of transducers) and display the at least the portion of the transducer-based device according to such model. In some embodiments encompassing at least FIG. 5A, a representation of the transducer-based device is provided by various elements of graphical representation 500. In some embodiments, the graphical interface depicts the transducer-based device as including a first domed portion 508a associated with a first domed portion of the transducer-based device (e.g., proximal portion 308a when having the first domed shape 309a) and a second domed portion 508b associated with a second domed portion of the transducer-based device (e.g., distal portion 308b having the second domed shape 309b). A separation graphical element 503 may be employed between the first and the second domed portions 508a, 508b in some embodiments, but may be omitted in other embodiments. Various other transducer-based devices may be depicted according to the instructions associated with block 602 in other embodiments. FIGS. 5A-5R, 5S-1, and 5S-2 (collectively FIG. 5) are presented in this disclosure in association with various embodiments. It is understood that each of these embodiments need not be associated with all of the FIGS. 5, and in some cases will only be associated with a subset of the FIG. 5.

In some embodiments, a plurality of graphical elements 501 (only two called out in FIG. 5A) are depicted (e.g., according to the instructions associated with block 602 or 602a, for example, in the case of FIG. 5A) among various elements of graphical representation 500. In various embodiments, each of the graphical elements 501 is respectively associated with a respective one of a plurality of transducer sets. According to some embodiments, each respective transducer set includes at least one transducer of a plurality of transducers included as part of the transducer-based device (e.g., transducer-based devices 200, 300, or 400, according to some embodiments), and each respective transducer set has at least one different transducer than another of the other transducer sets. In various particular embodiments, each respective transducer set has at least one different transducer than each of the other transducer sets.

FIG. 5B shows the graphical interface in which the display instructions have been configured to cause (for example, in response to user input via an input-output device system such as 120, 320) the three-dimensional graphical representation of the transducer-based device to be manipulated so as to be viewed from a different viewing angle than that shown in FIG. 5A. It is noted that three-dimensional representations of at least a portion of a transducer-based device are shown in FIGS. 5A, 5B, 5C, 5D, 5G, 5H, 5K, and 5M, according to some embodiments. Two-dimensional representations of at least a portion of a transducer-based device are shown in FIGS. 5E, 5F, 5I, 5J, 5L, 5N, 5O, 5P, 5Q, 5R, 5S-1, and 5S-2, according to some embodiments.

Referring to some embodiments encompassing FIG. 5A, each of at least some of the graphical elements 501 is provided by a respective one of a plurality of transducer graphical elements 502 that include at least a first transducer graphical element 502a, a second transducer graphical element 502b, and a third transducer graphical element 502c (e.g., all the transducer graphical elements forming part of a group of transducer graphical elements 502). In some embodiments, each transducer graphical element 502 is associated with, or corresponds to, a single respective transducer (e.g., a transducer 220, 306, or 406 in some embodiments) of the transducer-based device (e.g., transducer-based device 200, 300, or 400, according to some embodiments). In some example embodiments, each transducer graphical element 502 is representative of a respective transducer (e.g., a transducer 220, 306, or 406, in some embodiments) of the transducer-based device (e.g., 200, 300, or 400, according to some embodiments). However, in some embodiments, multiple graphical elements may be associated with, or correspond to, a single respective transducer of the transducer-based device. In some embodiments, each transducer may be identified by one or more graphical elements that do not represent the physical appearance of the transducer. For instance, a graphical user interface may include a text-based identifier, such as “A:1” to identify to a user a transducer in a first row and a first column in a grid of transducers, or “B:2” to identify to the user a transducer in a second row and a second column in a grid of transducers. In some example embodiments, each transducer graphical element 502 is representative of a location or position of a respective transducer of the transducer-based device. In some embodiments, the graphical representation 500 includes a first spatial relationship between the transducer graphical elements 502 that is consistent with a second spatial relationship between the corresponding transducers associated with the transducer graphical elements 502. For example, in some embodiments, the transducer graphical elements 502 in the three-dimensional graphical representation 500 in FIGS. 5A, 5B may exhibit a same spatial relationship that the transducers 306 exhibit in the transducer-based device 300 in FIGS. 3C and 3D. Or, in some embodiments, the transducer graphical elements 502 in other graphical representations 500 in others of FIG. 5 may exhibit a respective or corresponding spatial relationship that the transducers 306 exhibit in the respective transducer-based device. In this regard, in some embodiments, the graphical representation 500 may include a first spatial relationship between the transducer graphical elements 502 that is consistent with a second spatial relationship between the corresponding transducers associated with the transducer graphical elements 502 when the corresponding transducers are arranged in a deployed configuration (e.g., such as FIGS. 3C and 3D). For example, an adjacent pair of transducers (e.g., an adjacent pair of first transducer 306a and second transducer 306b, an adjacent pair of second transducer 306b and third transducer 306c, or an adjacent pair of first transducer 306a and third transducer 306c) arranged according to the second spatial relationship may correspond to an adjacent pair of the transducer graphical elements 502 arranged according to the first spatial relationship. In some embodiments, each particular depicted transducer graphical element 502 is shown having a shape that is consistent with the particular transducer (or portion thereof) that the particular transducer graphical element 502 is representative of.

In some example embodiments, graphical elements 501 may include alternate or additional forms. For example, FIG. 5C shows an example embodiment in which each of at least some of the graphical elements 501 are provided by a respective one of a plurality of between graphical elements 504 including a first between graphical element 504a and a second between graphical element 504b (e.g., all the between graphical elements collectively referred to as between graphical elements 504). The graphical elements are referred to as “between graphical elements” since they may be respectively located between, e.g., a respective group, or pair, in some embodiments, of adjacent transducer graphical elements 502, according to some embodiments. FIG. 5D shows an embodiment of the graphical interface in which the display instructions have been configured to cause (for example, in response to a user input via input-output device system 120 or 320) the depiction of the transducer-based device to be manipulated so as to be viewed from a different viewing angle than that shown in FIG. 5C.

In some embodiments, between graphical elements 504 are shown in addition to various ones of the transducer graphical elements 502 shown in FIGS. 5A and 5B. In some embodiments, between graphical elements 504 are provided separately or with other embodiments of graphical elements 501. In various embodiments, each of the between graphical elements 504 is associated with a set of at least two (e.g., a group) of the transducers of the transducer-based device. In some example embodiments, each of the between graphical elements 504 is associated with a pair of transducers in the transducer-based device. In some example embodiments, each between graphical element 504 is associated with a space between a respective pair of transducers in the transducer-based device. In some example embodiments, each between graphical element 504 is associated with a space between a respective pair of adjacent ones of the transducers in the transducer-based device. In some example embodiments, each between graphical element 504 is associated with a space between a respective pair of adjacent ones of the transducer graphical elements 502 in the graphical representation 500.

In some embodiments, selection of a between graphical element 504 may allow an efficient selection of multiple transducer graphical elements 502. For example, with reference to FIG. 5C, if between graphical element 504a is selected by a user interacting with the graphical interface of FIG. 5C, such selection may cause adjacent transducer graphical elements 502a and 502b to be selected. In some embodiments associated with FIG. 5C, if between graphical element 504a is selected by a user interacting with the graphical interface of FIG. 5C, such selection may cause adjacent transducer graphical elements 502a and 502b to be concurrently selected. In some embodiments, such a selection of a between graphical element 504 may cause selection of the corresponding pair of transducer graphical elements 502 for bipolar ablation performed by the physical transducers (e.g., transducers 220, 306, or 406) corresponding to the pair of transducer graphical elements 502. In some embodiments, such a selection of a between graphical element 504 may cause selection of the corresponding pair of transducer graphical elements 502 for concurrent activation of the physical transducers (e.g., transducers 220, 306, or 406) corresponding to the pair of transducer graphical elements 502. In this regard, a single transducer may be associated with multiple graphical elements, according to some embodiments. For instance, with respect to FIG. 5C, the physical transducer 306 that corresponds to transducer graphical element 502a may be associated not only with transducer graphical element 502a, but also each of its connected between graphical elements 504, in some embodiments. Further, although various ones of FIG. 5 illustrate a one-to-one correspondence between transducer graphical element 502 and transducer 306, such a one-to-one correspondence is not necessary in some embodiments. For instance, a transducer 306 may be represented by a symbol or other graphical depiction, which may be represented as a cluster of graphical elements, such cluster being considered to represent the single transducer 306, in some embodiments.

In some embodiments, first transducer graphical element 502a (e.g., FIG. 5C) is associated with, or corresponds to, a first transducer (e.g., first transducer 306a, e.g., FIG. 3D) of the transducer-based device, second transducer graphical element 502b is associated with, or corresponds to, a second transducer (e.g., second transducer 306b) of the transducer-based device, and third transducer graphical element 502c is associated with, or corresponds to, a third transducer (e.g., third transducer 306c) of the transducer-based device. In some embodiments, the first between graphical element 504a is associated with a first space that is between the first and the second transducers and the second between graphical element 504b is associated with a second space that is between the second and the third transducers. In some embodiments, the first space is a space that is not associated with any physical part of the transducer-based device (e.g., first space 350) and the second space is a space that is associated with a physical part of the transducer-based device (e.g., second space 360). In some embodiments, each of the first and the second between graphical elements 504a, 504b is associated with a space that does not include a transducer of the transducer-based device. In some embodiments, each of the first and the second between graphical elements 504a, 504b is associated with a space that does not include any transducer. It is understood that a “space” or “region of space” need not be a vacant space but can include physical matter therein.

In some embodiments, the first between graphical element 504a is positioned between the second and the first transducer graphical elements 502b, 502a among the graphical representation 500. In some embodiments, the second between graphical element 504b is positioned between the second and the third transducer graphical elements 502b, 502c among the graphical representation 500. In some embodiments, the second and the first transducer graphical elements 502b, 502a are adjacent transducer graphical elements. In some embodiments, the second and the first transducer graphical elements 502b, 502a are associated with adjacent transducers (e.g., transducers 306b, 306a). In some embodiments, the second and the third transducer graphical elements 502b, 502c are adjacent transducer graphical elements. In some embodiments, the second and the third transducer graphical elements 502b, 502c are associated with adjacent transducers (e.g., transducers 306b, 306c). In some embodiments, the first and the third transducer graphical elements 502a, 502c are adjacent transducer graphical elements. In some embodiments, the first and the third transducer graphical elements 502a, 502c are associated with adjacent transducers (e.g., transducers 306a, 306c). In other example embodiments, other spatial relationships exist between the transducer graphical elements 502 and the between graphical elements 504 in the graphical representation 500.

The transducer graphical elements 502, the between graphical elements 504, or both may have different sizes, shapes or forms than those shown in the illustrated embodiments. In some embodiments, at least one particular one of the transducer graphical elements 502 may be depicted with a different shape, size, or form than the respective one of the shape, size, or form of the respective portion of the particular transducer to which the particular one of the transducer graphical elements 502 corresponds. In some embodiments, different ones of the between graphical elements 504 may be depicted with different shapes, sizes, or forms.

With reference to various ones of FIGS. 5, at least a portion of the transducer graphical elements 502 and at least a portion of the between graphical elements 504 are arranged in a plurality of rows 510 (two called out in FIG. 5C) and a plurality of columns 512 (two called out in FIG. 5C). In some embodiments, each row 510 corresponds to a respective one of number “0”, “1”, “2”, “3”, “4”, “5”, “6”, “7”, “8”, “9”, “10”, and “11”, and each column 512 corresponds to a respective one of letters “A”, “B”, “C”, “D”, “E”, “F”, “G”, “H”, “I”, “J”, “K”, “L”, “M”, “N”, “O”, “P”, “Q”, “R”, “S”, and “T”, each of the numbers and letters used as part of the unique identifier 513 (only two called out with reference numeral 513 in FIG. 5C with respect to D:8 and E:9) of each transducer graphical element 502. In some embodiments, the plurality of rows 510 and columns 512 correspond to a condition in which structure 308 is in the deployed configuration. In some embodiments, a portion of each of the columns 512 may correspond to a space associated with a physical portion of the transducer-based device (e.g., an elongate member 304). In some embodiments, each of the columns 512 may correspond to at least a portion of the transducers located on a particular elongate member (e.g., an elongate member 304) of a transducer-based device. In some embodiments, at least one of the columns 512 may include at least one transducer graphical element 502 having a shape that is different than the respective shape comprised by any of the transducer graphical elements 502 included in at least one other of the columns 512. For example, the “A” column 512 includes a transducer graphical element 502 identified as “A:10” that has a shape that is different than any of the transducer graphical elements 502 comprised by at least one of the other columns 512. In some embodiments, at least a first one of the rows 510 may include identically shaped transducer graphical elements 502 (e.g., row 510 that includes transducer graphical elements 502 identified as “A:6”, “B:6”, “C:6”, “D:6”, “E:6”, “F:6”, “G:6”, “H:6”, “I:6”, “J:6”, “K:6”, “L:6”, “M:6”, “N:6”, “O:6”, “P:6”, “Q:6”, “R:6”, “S:6”, and “T:6” (not all of which are shown in FIG. 5A due to the presented perspective)), and at least a second one of the rows 510 may include differently shaped transducer graphical elements 502 (e.g., row 510 that includes transducer graphical elements 502 identified as “A:10”, “B:10”, “C:10”, “D:10”, “E:10”, “F:10”, “G:10”, “H:10”, “I:10”, “K:10”, “L:10”, “M:10”, “N:10”, “O:10”, “P:10”, “Q:10”, “R:10”, and “S:10”).

In some example embodiments, a portion of each of the rows 510 may correspond to spaces not associated with any physical portion of the transducer-based device (e.g., space 350 between adjacent ones of the elongate members 304). In other example embodiments, different numbers of transducer graphical elements 502 and different numbers and spatial arrangements of between graphical elements 504 may be depicted in the graphical representation. In other example embodiments, different numbers and spatial arrangements of rows 510 and columns 512 may be depicted in the graphical representation. In various embodiments, each of the between graphical elements (e.g., between graphical elements 504) depicted in the graphical representation may be representative of a respective physical path extending between a respective pair of transducers of the transducer-based device. Each of the physical paths may extend over a physical surface of the transducer-based device or over a portion of an opening defined by a physical surface of the transducer-based device. In some embodiments like FIG. 5C, each between graphical element 504 may be representative of a respective path in real world space, the path extending (e.g., as the crow flies in some embodiments) between the respective transducers associated with the adjacent pair of transducer graphical elements 502 that the between graphical element 504 extends between. In some embodiments like FIG. 5C, each adjacent pair of the transducer graphical elements 502 may be provided along a row 510 (two called out in FIG. 5C) of the graphical elements 501, along a column 512 (two called out in FIG. 5C) of the graphical elements 501, or diagonally between a row 510 and a column 512.

Referring back to FIGS. 5A, 5B, the plurality of rows 510 and the plurality of columns 512 are depicted as a three-dimensional arrangement in the graphical representation. In some embodiments, at least two of the plurality of columns 512 are depicted in the graphical representation extending along respective directions that converge with respect to one another. In some embodiments, at least two of the plurality of columns 512 are depicted in the graphical representation extending along non-parallel directions and at least two of the plurality of rows 510 are depicted extending along parallel directions. In some embodiments, the rows 510 and the columns 512 are depicted in the graphical representation in an arrangement in which the columns 512 are circumferentially arranged. In some embodiments, the rows 510 and the columns 512 are depicted in the graphical representation in an arrangement having a generally spherical shape. The plurality of columns 512 may be depicted like lines of longitude, and the plurality of rows 510 may be depicted like lines of latitude. Although the rows 510 and columns 512 are illustrated in FIGS. 5A-5D as circumferential lines (like lines of longitude and latitude), such rows 510 and columns 512 can take other forms, as shown, for example, in FIGS. 5E and 5F, discussed in more detail below, according to some embodiments.

The graphical representation instructions associated with block 602 may include instructions (e.g., instructions responsive to a user input made via an input-output device system) configured to vary the depiction of the portion of the transducer-based device between a three-dimensional representation (e.g., as depicted in various ones of at least FIGS. 5A, 5B, 5C, and 5D, according to some embodiments) and a two-dimensional representation (e.g., as depicted by at least FIG. 5E or 5F, according to some embodiments). Various two-dimensional representations are possible in various embodiments. For instance, the plurality of transducer graphical elements 502 may be arranged in the graphical representation 500 in a particular spatial distribution representing the three-dimensional distribution of transducers (e.g., 220 or 306) distorted onto a two-dimensional plane to form the two-dimensional representation. In this regard, in some embodiments, the two-dimensional representation of the three-dimensional distribution of transducers (e.g., 220 or 306) distorted onto a two-dimensional plane is not merely an isometric or other perspective view of the three-dimensional distribution of transducers, as such an isometric or other perspective view would be considered a three-dimensional representation, such as that shown in various ones of FIGS. 5A, 5B, 5C, and 5D. The two-dimensional representation may be generated according to the display instructions according to a conformal map or projection, such as a Mercator map or projection, a transverse Mercator map or projection, or other three-dimensional-to-two-dimensional map or projection, known in the art, according to some embodiments. According to various embodiments, a conformal mapping is a function that preserves local angles. For example, according to some embodiments, when a particular spatial relationship between the plurality of transducers 306 is conformally mapped to the graphical representation 500, an angle defined between a group of transducers (e.g., 306) according to the particular spatial relationship is preserved between the corresponding group of transducer graphical elements 502. In some embodiments, the two-dimensional representation need not be a projection or map from a three-dimensional model, and may merely be any two-dimensional representation, e.g., including an arrangement of transducers.

The two-dimensional representation depicted in FIG. 5E, according to some embodiments, represents the first domed portion 508a (e.g., shown in FIGS. 5C, 5D) of the depicted transducer-based device as first Mercator projection 518a and the second domed portion 508b (e.g., shown in FIGS. 5C, 5D) of the depicted transducer-based device as a second Mercator projection 518b. The first and the second Mercator projections 518a and 518b advantageously allow for simultaneous viewing of all the transducer graphical elements 502 and the between graphical elements 504. Columns 512 and rows 510 are depicted two-dimensionally in FIG. 5E. In some embodiments, separation graphical element 503 is also depicted in a two-dimensional configuration.

As discussed above, other two-dimensional representations may be implemented and may be user-selectable for viewing. For example, FIG. 5F illustrates a transverse Mercator projection employed according to some embodiments. In FIG. 5F, the transverse Mercator projection includes two portions 518c, 518d, each of the portions 518c, 518d representative of a respective one of first and second domed portions 508a and 508b in the corresponding three-dimensional representation. In FIG. 5F, portion 518d of the transverse Mercator projection is shown as two parts, each part at least depicting the transducer graphical elements 502 in a respective one of two parts of the domed portion 508b. In FIG. 5F, portion 518c is representative of first domed portion 508a. In some embodiments, various ones of the columns 512 radiate outwardly radially or quasi-radially from particular ones of a plurality of pole regions 511a and 511b represented in the graphical representation 500. In some embodiments, various ones of the rows 510 are circumferentially arranged about particular ones of a plurality of pole regions 511a and 511b.

In some embodiments, at least some of the between graphical elements 504 are not shown in various ones of the displayable two-dimensional representations. For example, in FIG. 5F, between graphical elements 504 have been selectively controlled, e.g., in response to user input, not to be visible among the graphical representation. In various embodiments, the transducer graphical elements 502 shown in each of the FIGS. 5E and 5F are arranged with respect to one another according to a spatial relationship that corresponds to a spatial relationship that the transducer graphical elements are arranged in the three-dimensional representations shown in various ones of FIGS. 5A, 5B, 5C, and 5D. In various embodiments, the transducer graphical elements 502 shown in each of the FIGS. 5E and 5F are arranged with respect to one another according to a spatial relationship that corresponds to a spatial relationship that particular transducers, which the transducer graphical elements 502 correspond to, are arranged with respect to one another when a supporting structure (e.g., structure 308) is in a deployed configuration.

Various computer-executable instructions may be configured to control various input element control functions (e.g., mouse drag functions, touch screen drag functions) between various operating modes such as rotating and panning modes. A rotating mode may be advantageously used for manipulation of a three-dimensional representation of a transducer-based device or other portions of the graphical representation 500 to allow for viewing one or more portions of the three-dimensional representation of the transducer-based device or various portions of the graphical representation 500 that were not previously viewable (e.g., a manipulation between the views shown in FIGS. 5A and 5B or a manipulation between the views shown in FIGS. 5C and 5D). In some embodiments, a panning mode or rotating mode may be advantageously used for manipulation of a two-dimensional representation of the transducer-based device or other portions of the graphical representation 500 to allow for viewing of different arrangements of various graphical elements in the representation of a transducer-based device or other portions of the graphical representation 500.

The graphical representation 500 displayed according to the instructions associated with block 602 in FIG. 6A is not limited to three-dimensional representations of a spatial distribution of the plurality of graphical elements 501 in the context of a display of a three-dimensional representation of a transducer-based device (e.g., as shown in FIGS. 5A, 5B, 5C, and 5D). Other entities may also be graphically depicted in a three-dimensional (or two-dimensional) manner. For example, FIG. 5G illustrates a graphical interface including a graphical representation 500 provided by the input-output device system 120, 320, according to some embodiments provided in accordance with graphical representation instructions associated with block 602 in FIG. 6A. In some embodiments, the graphical representation 500 in FIG. 5G includes a three-dimensional graphical representation of at least a portion of a transducer-based device 500a that is similar to that depicted in FIGS. 5A, 5B, 5C, and 5D. The graphical representation 500 of FIG. 5G also shows, e.g., according to program instructions associated with block 602b in FIG. 6A in some embodiments, a three-dimensional representation of an envelope 550 that graphically represents at least a portion of a bodily cavity (e.g., the atrium of a heart in this depicted embodiment) in which the transducer-based device may be deployed. According to various embodiments, envelope 550 may be generated with the use of a catheter-device-location tracking system or navigation system (e.g., an electro-potential-based navigation system or an electromagnetic-based tracking system). Some examples of such a location tracking system or navigation system can be found at least in U.S. Patent Application Publication No. 2021/0353370, published Nov. 18, 2021 (Moisa). The catheter-device-location tracking system may, in some embodiments, be configured to provide location information derived from a plurality of location signal sets provided to the data processing device system 110, 310. According to various embodiments, the location information may indicate a plurality of locations in a bodily cavity in response to movement of at least part of a transducer-based device (e.g., transducer-based device 200, 300, or 400, in some embodiments) in the bodily cavity. For example, with respect to at least FIG. 2, at least a portion of the transducer-based device (e.g., transducer-based device 200, 300, or 400, according to some embodiments) may be moved or progressed through a sequence of locations in a chamber of the heart or other bodily cavity in the presence of an electric field set (e.g., one or more electric fields generated by the external electrodes (not shown)) or a magnetic field set (e.g., one or more magnetic fields generated by magnetic field generation sources (not shown)). As the portion of the transducer-based device is moved through the sequence of locations, at least some of the catheter's transducers may be configured to generate each location signal set as detected strengths of the respective field(s), which its data processing device system 110, 310 may then be configured to utilize to generate a three-dimensional location of the at least the portion of the catheter (e.g., transducer-based device 200, 300, or 400, according to some embodiments) or its transducers for the respective location in the sequence of locations, according to some embodiments. In this regard, the portion of the transducer-based device may then be moved along the surface of the bodily cavity while providing the location signal sets, which may be used to generate, e.g., according to program instructions associated with block 602b in some embodiments, the envelope 550 in FIG. 5G representative of the surface of the bodily cavity. Intra-cardiac information (discussed in further detail below) may be recorded (e.g., simultaneously) with the location signal sets and correlated to the generated location information in some embodiments. The recorded intra-cardiac information may be represented on the envelope 550, according to some embodiments.

In some embodiments, the graphical representation of the envelope representing a three-dimensional representation of an interior volume of the bodily cavity may be produced with respect to a graphical representation of a pre-existing image or model, such as a CT scan, of the cavity, further assisting the operator to understand the location of the transducer-based device, as well as potential future desired movements of the transducer-based device FIG. 5H illustrates an example of a display of graphical representation 500 including an envelope 551 provided at least in part by a CT scan of the cavity, e.g., according to some embodiments associated with block 602b in FIG. 6A. In some embodiments, the graphical representation 500 may include overlapped and registered envelopes 550 and 551.

In some embodiments, the graphical representation instructions of block 602, block 602a, or block 602b may be configured to cause display of information in addition to or other than (a) a graphical representation of at least part of a transducer-based device such as graphical elements associated with transducers, or (b) a representation of at least a portion of a bodily cavity. In some embodiments, program instructions associated with block 602 (or block 602a, block 602b, or both blocks 602a and 602b) in FIG. 6A may include instructions (e.g., input or acquisition instructions included in a program) configured to cause the data processing device system (e.g., data processing device systems 110 or 310) to acquire or receive intra-cardiac information. The graphical representation instructions associated with block 602 (and, in some embodiments, also block 602a, block 602b, or both blocks 602a and 602b) may cause display of various representations of the intra-cardiac information or derivative thereof. Intra-cardiac information can take various forms, including, but not limited to, e.g., electrical information or a derivation thereof (e.g., electrical potential information, such as intra-cardiac electrogram information; electrical impedance information, such as fluidic or non-fluidic cardiac tissue impedance information; electrical conductivity information, such as fluidic or non-fluidic cardiac tissue electrical conductivity), thermal information or a derivation thereof (e.g., temperature information), fluid property information or a derivation thereof (e.g., blood flow information, blood pressure information), force information or a derivation thereof (e.g., contact information), and mapping information or a derivation thereof (e.g., electrical mapping; physical feature mapping, such as anatomical feature mapping). In various embodiments, intra-cardiac information may be related to any physiological parameter information related to a heart chamber. In various embodiments, intra-cardiac information may include any information related to, or resulting from an interaction with intra-cardiac tissue. By way of non-limiting example, interaction with intra-cardiac tissue may include an interaction made by way of a diagnostic procedure or treatment procedure.

Intra-cardiac information may be acquired or received by various methods and from various device systems. For example, intra-cardiac information may be received or acquired via data sampling performed by a transducer-based device system (e.g., which may be at least part of the data input-output device system 120, 320) deployed externally from an intra-cardiac chamber or cavity (e.g., outside the chamber or cavity or outside a body including the chamber or cavity). By way of non-limiting example, various “external” transducer-based device systems may include various fluoroscopy device systems, ultra-sound device system, magnetic resonance device systems, computerized tomography device systems, and transthoracic electrocardiogramapping device systems. In some embodiments, reception or acquisition of the intra-cardiac information may be via data sampling performed by a transducer-based device system (e.g., which may be at least part of the data input-output device system 120, 320) deployed internally to an intra-cardiac chamber or cavity. By way of non-limiting example, various transducer-based device systems that may be internally deployed within an intra-cardiac chamber include by way of non-limiting example transducer-based device systems 200, 300, 400 where data may be sampled by each of one or more transducers of the transducer-based device system, a portion of the transducer-based device system including the one or more transducers positionable in a cardiac chamber during the sampling. Various transducer-based devices may include various intravascularly deployable or percutaneously deployable catheter device systems. Various transducer-based device systems may include detection capabilities, mapping capabilities, diagnostic capabilities, treatment capabilities, or any combination thereof.

The displaying of the graphical representation according to the computer-executable instructions associated with block 602 (and, in some embodiments, also block 602a, block 602b, or both blocks 602a and 602b) may, in some embodiments, include causing displaying of a graphical representation of the intra-cardiac information or derivative thereof. Various embodiments may process or analyze (e.g., according to the instructions associated with block 602) the transducer data received by the data processing device system in order to, for example, generate and cause the displayed graphical representation 500 to include the intra-cardiac information. Various embodiments may process or analyze the transducer data received by the data processing device system in order to, for example, generate and possibly cause the displayed graphical representation 500 to include a map of the intra-cardiac information. In various embodiments, the data is sampled by a transducer-based device system from a plurality of locations in a cardiac chamber, which may allow for a mapping of each of a plurality of parts or values of the intra-cardiac information (which may represent a sensed tissue electrical characteristic or other information) to a respective one of the plurality of locations in the cardiac chamber. In some of these various embodiments, the graphical representation instructions associated with block 602 may be configured to cause an input-output device system (e.g., 120, 320) to display the plurality of parts of the intra-cardiac information with a first spatial relationship that is consistent with a second spatial relationship between the plurality of locations in the cardiac chamber (e.g., a map of the parts of the intra-cardiac information may be displayed). In some embodiments, the transducer-based device includes a plurality of transducers (e.g., transducer-based device 200, 300) and the sampled data may be sampled concurrently from the plurality of locations of the transducers in the cardiac chamber.

An example of a display of a graphical representation that at least depicts intra-cardiac information according to various embodiments (such as those represented by block 602 in FIG. 6A) may be a mapping locating the position of the ports of various bodily openings positioned in fluid communication with a cardiac chamber. For example, in some embodiments, it may be desired to determine intra-cardiac information indicating the locations of various ones of the pulmonary veins or the mitral valve that each interrupts an interior surface of an intra-cardiac cavity such as a left atrium.

In some example embodiments, the mapping is based at least on locating such bodily openings by differentiating between fluid and tissue (e.g., tissue defining a surface of a bodily cavity). There are many ways to differentiate tissue from a fluid such as blood or to differentiate tissue from a bodily opening in case a fluid is not present. Four approaches may include by way of non-limiting example:

1. The use of convective cooling of heated transducer elements by fluid. A slightly heated arrangement of transducers that is positioned adjacent to the tissue that forms the interior surface(s) of a bodily cavity and across the ports of the bodily cavity will be cooler at the areas which are spanning the ports carrying the flow of fluid.

2. The use of tissue impedance measurements. A set of transducers positioned adjacently to tissue that forms the interior surface(s) of a bodily cavity and across the ports of the bodily cavity can be responsive to electrical tissue impedance. Typically, heart tissue will have higher associated tissue impedance values than the impedance values associated with blood.

3. The use of the differing change in dielectric constant as a function of frequency between blood and tissue. A set of transducers positioned around the tissue that forms the interior surface(s) of the atrium and across the ports of the atrium monitors the ratio of the dielectric constant from 1 kHz to 100 KHz. Such can be used to determine which of those transducers are not proximate to tissue, which is indicative of the locations of the ports.

4. The use of transducers that sense force (e.g., force or pressure sensors). A set of force detection transducers positioned around the tissue that forms the interior surface of the bodily cavity and across the bodily openings or ports of the bodily cavity can be used to determine which of the transducers are not engaged with the tissue, which is indicative of the locations of the ports.

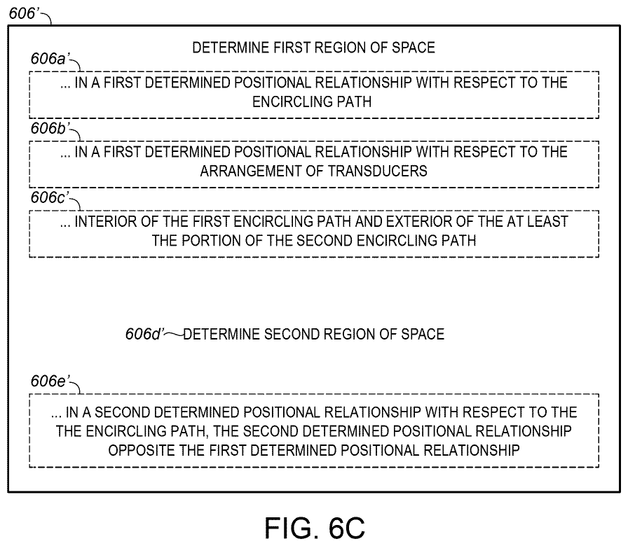

The graphical interface of FIG. 51 includes a graphical representation 500 including various regions 525a, 525b, and 525c (e.g., part of a plurality of regions collectively referred to as regions 525) added to the graphical representation 500 shown in FIG. 5E. The regions 525 may be displayed according to the instructions associated with block 602 (and, in some embodiments, also block 602a, block 602b, or both blocks 602a and 602b) in FIG. 6A in some embodiments. Although, such regions 525 may be displayed at other times or according to other instructions. In some embodiments, the graphical interface depicted in FIG. 51 is generated after the transducer-based device is received in a bodily cavity having various anatomical features of interest and flow-based mapping techniques are employed.

Techniques for flow-based mapping techniques are disclosed in U.S. Patent Application Publication No.: 2008/0004534, published Jan. 3, 2008 (Gelbart et al.). In various embodiments associated with various ones of FIGS. 5, the anatomical features of interest are ports of a mitral valve and various pulmonary veins positioned in fluid communication with an intra-cardiac cavity (e.g., a left atrium in some embodiments). In these various embodiments, the transducers of the transducer-based device are distributed adjacent respective regions in the intra-cardiac cavity that can include relatively lower blood flow regions (e.g., adjacent a tissue surface of the intra-cardiac cavity) and relatively higher flow regions (e.g., over the ports of the intra-cardiac cavity). It is noted that relatively lower blood flow regions in the intra-cardiac cavity may occur when a transducer is positioned in contact with a tissue surface to restrict blood flow at the contacted tissue. In some example embodiments, a relatively large number of transducers in the distribution advantageously allows for each of the transducers to be positioned adjacent their corresponding regions with little or no repositioning of the transducer-based device thereby facilitating an obtaining of transducer-based data concurrently from multiple locations in the bodily cavity. In some embodiments, different regions of an intra-cardiac cavity (including for example, ports such as pulmonary veins) may be graphically represented based on data provided by a catheter-device-location tracking system or navigation system.

One or more of the above-discussed mapping procedures may be implemented according to instructions associated with block 602 to display a graphical representation 500 that includes intra-cardiac information that indicates at least a portion of one or more anatomical features based at least on an analysis of various transducer data that may be acquired or received. In some of these embodiments, the one or more anatomical features are the ports of various bodily openings (e.g., pulmonary veins, left atrial appendage, mitral valve) positioned in fluid communication with the intra-cardiac cavity and the transducer data includes data containing various blood flow data within the bodily cavity. In some embodiments, the sampled data may be temperature data and the graphical representation 500 includes a graphical representation of at least some of the temperature data or a derivation thereof (e.g., a map of temperature distribution in the cardiac chamber). For example, in various embodiments in which the use of convective cooling of heated transducer elements by fluid is employed to distinguish blood flow adjacent to the tissue that forms the interior surface(s) of a cardiac chamber from blood flow across the ports of the cardiac chamber, temperature data associated with the convective cooling can be sampled and displayed to provide the graphical representation of the intra-cardiac information. In FIG. 51, the relatively large region 525a (e.g., shown as two parts in this particular orientation of the two-dimensional representation) is associated with the mitral valve, region 525b is associated with the left atrial appendage, and regions 525c are associated with various pulmonary vein groups. Each of the regions 525 is depicted in the graphical representation 500 with a graduated pattern, according to some embodiments. A graduated pattern can be employed to indicate various regions in the graphical representation corresponding to different regions of flow in the intra-cardiac cavity. The identified regions 525 may be identified by any suitable methods including the use of gray-scale patterns, different colors, different opacities, different intensities and different shapes. It is understood that other embodiments may employ other techniques to identify regions in the graphical representation corresponding to a desired anatomical feature. For example, transducer-based data containing blood and tissue impedance information may be employed to determine regions 525 according to some embodiments.

Identification of the regions 525, which may represent anatomical features, may be motivated for various reasons. For example, in embodiments in which transducers of a transducer-based device are activated to treat, diagnose, or investigate various regions in a bodily cavity, the mapping of various regions 525 and their spatial relationship relative to one another may impact the efficacy of the treatment, diagnostic, or investigative procedure. For example, in situations in which at least some of the transducers of a transducer-based device are employed to ablate various regions within an intra-cardiac cavity (e.g., to treat atrial fibrillation), ablation of tissue surrounding one or more pulmonary veins may be employed in a procedure called pulmonary vein isolation. Identification of various ones of the regions 525c in the graphical representation along with their spatial relationship with various ones of the transducers at various times may be employed to determine specific tissue regions to ablate. It is noted that the identification of regions such as regions 525 is not limited to embodiments employing graphical representations 500 including various two-dimensionally depicted entities, as graphical representations 500 including various three-dimensionally depicted entities may be employed.

Without limitation, other forms of intra-cardiac data that may form part of the graphical representation 500 may include pressure data (e.g., blood pressure data, contact pressure data), electrophysiological activation timing data, isochronal data, propagation data, electrophysiological isopotential data, and other electrophysiological voltage data. Without limitation, various maps of intra-cardiac data may include tissue contact maps (e.g., contact maps inferred from flow data, impedance data, conductivity data, which may map an interior tissue surface region of a cardiac chamber), activation maps indicating the local activation times associated with a particular cardiac event, isochronal maps where contour lines may delineate regions of equal activation times associated with a particular cardiac event, propagation maps providing a dynamic representation of the moving activation wave-front associated with a particular cardiac event, isopotential maps, and various other voltage maps associated with intra-cardiac electrical activity. Various representations (e.g., maps) of intra-cardiac information may include portions corresponding to values measured at specific locations within an intra-cardiac cavity and portions corresponding to values that are interpolated (for example, interpolated from values measured at specific locations within an intra-cardiac cavity).

In some embodiments, intra-cardiac information is depicted in the graphical representation statically or relatively statically. That is, the displayed intra-cardiac data remains unaltered or relatively unaltered during a defined display period. In some embodiments, intra-cardiac information is depicted in the graphical representation 500 such that variances in the intra-cardiac information are shown occurring over a defined display period.

It is noted that in various example embodiments, such as those associated with various ones of FIGS. 5, at least some of the graphical elements 501 (e.g., transducer graphical elements 502, between graphical elements 504) may be depicted as overlaid or superimposed on a depiction of the acquired intra-cardiac information. In various embodiments, various ones of the graphical elements 501 (e.g., various ones of the transducer graphical elements 502) are depicted with a transparent, semi-transparent, or translucent appearance that allows a user to view regions of the intra-cardiac information that underlie each of the various ones of the graphical elements 501 or visual changes in the regions of the intra-cardiac information that underlie each of the various ones of the graphical elements 501. This configuration can be especially advantageous when one hundred, two hundred, or even more transducers are employed percutaneously to sample or gather the intra-cardiac information from a cardiac chamber. A graphical representation 500 that employs a similar, equal, or greater number of graphical elements 501 (e.g., transducer graphical elements 502, between graphical elements 504 or both transducer graphical elements 502 and between graphical elements 504) may obstruct a required viewing of the displayed intra-cardiac information, especially when transducer graphical elements 502 having a shape consistent with the shapes of corresponding ones of the transducers are employed or when transducer graphical elements having distorted appearances (e.g., enlarged distorted appearances described above) are employed. These situations may be effectively mitigated by the use of various graphical elements 501 having a transparent, semi-transparent, or translucent appearance.

Having described examples of the graphical representation 500 displayed according to the instructions associated with block 602 in FIG. 6A, the processing of user input received according to program instructions associated with block 604, which may facilitate selection, in some embodiments, of one or more graphical elements or their corresponding transducers, will be described, according to various embodiments. In some embodiments associated with block 604, such user input may be received via an input-output device system, such as input-output device system 120, 320. In some embodiments associated with at least block 604a, the user input may indicate a selected graphical element set. For example, a user may interact with a graphical interface associated with one or more of the various FIGS. 5, e.g., by using a mouse, a touch screen, a keyboard, or any other manner of interacting with a graphical interface, in order to select one or more graphical elements 501, 502, or 504, according to some embodiments. Such a selected graphical element set may be associated with an arrangement of transducers, and in some embodiments associated with block 604a, the arrangement of transducers may be distributed around, surrounding, or distributed along a path that surrounds a particular region of space. For example, FIG. 5M may illustrate a state in which a user has selected, e.g., by using a combination mouse click and movement or by using a touch screen, the outer ring of “hatched” (i.e., hatched to indicate selection thereof) graphical elements 545a (two elements of which are called out in FIG. 5M) as the selected graphical element set. (The inner ring of “hatched” graphical elements 545b will be discussed in more detail below.) In this regard, the “hatched” graphical elements 545a may be associated with an arrangement of transducers (e.g., transducers 220, 306, or 406 in some embodiments) that are distributed around a particular region of space. With respect to the example of FIG. 5M, the particular region of space may be the real-world space that corresponds to the graphical region 541 inside the ring of “hatched” graphical elements 545a, according to some embodiments.

In some embodiments, the user input received according to program instructions associated with block 604 may be provided by an indication, associated with the user input, of at least one path. Such a path may be utilized to indicate a region of interest in the graphical representation (e.g., graphical representation 500). For instance, in some embodiments, a user may interact with a graphical interface of the input-output device system 120, 320, such as one of those in the various FIG. 5, to select, define, draw, or otherwise indicate a path around a graphical representation of at least a portion of a bodily cavity, at least a portion of a transducer-based device, at least a group of transducers, or some combination of these items, to indicate or define a region that may be of interest to the user. According to some embodiments associated with block 604b and block 604c, each of one or more of such paths may include an encircling path. In this regard, instructions associated with block 604 and at least block 604b and block 604c in some embodiments may configure a data processing device system (e.g., 110, 310) to cause reception, via the input-output device system 120, 320 of user input indicating or defining an encircling path in or among the graphical representation 500. In some embodiments, the encircling path is indicated or defined in or among a graphical representation of at least part of transducer-based device (e.g., transducer-based device 200, 300, or 400, according to some embodiments) depicted in the graphical representation 500.

In some embodiments, e.g., associated with some embodiments of block 604a in FIG. 6A, the encircling path may be indicated in or among a set of selected graphical elements (e.g., graphical elements 502 in some embodiments) associated with an arrangement of transducers distributed around, or distributable around, a particular region of space. The selected graphical elements may be selected in response to the encircling path being produced on or among such graphical elements, according to some embodiments. Other embodiments associated with block 604a may cause selection of the selected graphical element set with other manners of user input besides the indication of an encircling path.

In some embodiments, the encircling path is indicated in or among a graphical representation of intra-cardiac information depicted in the graphical representation 500. In some embodiments (e.g., per block 604b1 in FIG. 6A), the encircling path is indicated around at least a region of a representation of at least a portion of a bodily cavity. In some embodiments, the encircling path is indicated in or among a graphical representation of a map of a bodily cavity (e.g., an anatomical or electro-anatomical map of a bodily cavity such as a cardiac cavity) depicted in the graphical representation 500. In some embodiments (e.g., per block 604b2 in FIG. 6A), the encircling path is indicated in or among at least some (e.g., at least a first group in some embodiments) of at least some of the displayed plurality of graphical elements 501. In some embodiments, the encircling path indicated by the received user input may be displayed, e.g., in or among at least a portion of the graphical representation (e.g., graphical representation 500), for example, to provide feedback to the user as to where the encircling path was drawn, selected, or otherwise indicated. In some embodiments, the graphical representation instructions (e.g., associated with block 602, in some embodiments) are configured to cause display, via the input-output device system (e.g., 120, 320), of the encircling path around the at least the above-mentioned region of the representation of the at least the portion of the bodily cavity. For instance, at least FIGS. 5N, 5O, 5P, 5Q, and 5R, discussed in more detail below, provide examples where an encircling path may be formed and displayed around a region of a representation of at least a portion of a bodily cavity.

The user input defining or indicating the encircling path may be motivated for different reasons. In some embodiments, the user input may define an encircling path to indicate a region within the graphical representation 500 that may be of interest. For example, the graphical representation 500 may include a graphical representation of a tissue surface within a bodily cavity, and a user may identify a particular area of the depicted tissue surface with user input indicating an encircling path. In some embodiments, the bodily cavity may be an intracardiac cavity and the particular area of interest of the depicted tissue surface may be one or more pulmonary veins, the encircling path surrounding at least part of the depicted one or more pulmonary veins. In some embodiments, the bodily cavity may be an intracardiac cavity and the particular area of the depicted tissue surface may be one or more pulmonary veins intended to undergo pulmonary vein isolation, the encircling path surrounding at least part of the depicted one or more pulmonary veins.

In some embodiments, graphical representation instructions (e.g., those associated with block 602 or otherwise) are configured to cause display, via the input-output device system 120, 320 of the encircling path among at least some of the displayed plurality of graphical elements 501. For example, as shown in FIG. 5J, an encircling path has been indicated or defined in the graphical representation 500, and, in some embodiments, such encircling path is displayed graphically as displayed path 530 in the graphical representation 500, in response to user input. In FIG. 5J, the displayed path 530 is a dotted line having a ring shape that surrounds, encircles, encloses, or is distributed around a region 525c, which graphically represents a pulmonary vein group according to some embodiments associated with FIG. 5J. In this regard, in some embodiments, the encircling path may be formed by user input from a mouse, touch screen, or other manner of input that draws or otherwise indicates the encircling path. Accordingly, although the encircling path may be drawn or otherwise indicated by the user input, such encircling path need not be displayed in the graphical representation (e.g., graphical representation 500), although in some embodiments, it may be represented graphically, such as by displayed path 530 in some embodiments.

According to various embodiments, graphical representation instructions (e.g., those associated with block 602 or otherwise) may be configured to cause display, via the input-output device system 120, 320 of the encircling path (e.g., which may be displayed as or represented by the displayed path 530 in some embodiments) distinctly from the displayed plurality of graphical elements 501. For example, in FIG. 5J, the displayed path 530 is shown as a discontinuous dotted path (e.g., a bread crumb trail) that is distinct in form from the graphical elements 501. At least FIGS. 5N, 5O, 5P, 5Q, and 5R, discussed in more detail below, provide other examples where an encircling path may be displayed distinctly from graphical elements. It is noted that the displayed path 530 is not limited to the form displayed in FIG. 5J and can include other discontinuous or continuous forms in various embodiments. Without limitation, in some embodiments, the displayed path 530 may be represented with any particular visual characteristic set that may distinguish at least part of the displayed path 530 from other parts of the graphical representation 500 (e.g., graphical elements 501) so that the displayed path is visible or apparent to a user. Without limitation, the displayed path 530 may include a continuous or discontinuous form that outlines or otherwise defines various shapes that include circular, elliptical, ovoid, square, rectangular, triangular, polygonal, and other regular and non-regular shapes.

In some embodiments, the encircling path, which may be represented by displayed path 530 in some embodiments, defines or identifies at least three or at least five non-colinear locations in the graphical representation 500. The defined or identified at least three or at least five non-colinear locations in the graphical representation 500 may define respective locations of a region of graphical space in the graphical representation 500 that is outlined by the displayed encircling path 530. For example, in FIG. 5J, the at least three or at least five non-colinear locations may represent at least three or at least five of the dots in the displayed path 530 that represent a curving portion of the encircling path. In some embodiments, if the graphical representation is a three-dimensional representation, the displayed version of the encircling path may appear as encircling in some perspectives, but in other perspectives, such as a side (e.g., in-plane) perspective of the displayed path 530, the displayed path 530 may appear linear in some embodiments, such that the displayed path 530 in some of these embodiments does not have non-colinear locations. In some embodiments, the displayed path 530 does not intersect any of the displayed graphical elements 501. In some embodiments, the displayed path 530 intersects at least one of the plurality of graphical elements 501. For example, in FIG. 5J, the displayed path 530 intersects the transducer graphical elements 502F1, 502L1, and 502F5.

Similarly, in at least some embodiments in which selected graphical elements are selected via user-input per some embodiments of block 604 and are associated with an arrangement of transducers (e.g., per block 604a), the arrangement of transducers may include at least three transducers that are not colinearly arranged with respect to one another, in some embodiments. For example, the transducers associated with transducer graphical elements 502F1, 502L1, and 502F5, which are intersected by displayed path 530 and, therefore, may be considered an arrangement of transducers whose associated graphical elements are user-input selected, are not colinearly arranged with respect to one another. Such transducers corresponding to transducer graphical elements 502F1, 502L1, and 502F5 may be considered a group of at least three transducers in the arrangement of transducers that are not colinearly arranged with respect to one another.

The user input indicating the encircling path (e.g., per blocks 604b and 604c) may take various forms according to some embodiments. In some embodiments, a user may make a mouse click, touch a touch screen, or may utilize any other form of user input, in order to select each of multiple individual locations of the encircling path to select, define, or identify the encircling path. In this regard, according to some embodiments, each location of the multiple locations in the graphical representations may be individually selected. In some embodiments, the multiple selected locations in the graphical representation 500 from part of the encircling path. In some embodiments, the multiple selected locations in the graphical representation 500 define or identify the encircling path. In some embodiments in which a user selects or identifies multiple distinct locations, the data processing device system 110, 310 may be configured by a program to define or fit an encircling path that intersects each of the multiple selected locations in the graphical representation 500. For example, in some embodiments, the data processing device system 110, 310 may define linear or arcuate path segments that circumferentially connect adjacent pairs of the multiple selected locations in the graphical representation 500. In some embodiments, the data processing device system 110, 310 may be configured by the program to indicate or define the encircling path by connecting successive selected ones of the multiple selected locations in the graphical representation 500. In some of embodiments in which the encircling path is displayed and multiple user-selected distinct locations are identified and then are joined, e.g., by the data processing device system, to form the encircling path, the displayed path 530 may be displayed in a continuous form, instead of the discontinuous form shown in FIG. 5J.

In some embodiments, the encircling path may be selected, defined, drawn, or otherwise indicated by user input that may include motion-based user input. For example, in some embodiments, a user may locate a mouse cursor over a particular location in the graphical representation 500, depress a mouse button at that time, and while continuing to depress the mouse button, move the mouse cursor over the graphical representation 500, the movement of the mouse cursor defining or indicating the encircling path. In some embodiments, the conclusion of the definition or indication of the encircling path corresponds to a release of the mouse button. It is noted that the use of a mouse cursor is described above merely for the convenience of discussion, and other embodiments may employ other forms of motion-based user input elements (e.g., sliding of contact across a touch screen or touch pad or the movement of other pointing-based interfaces) or other forms of indicators employed by various motion-based user input elements. In addition, other or additional user input than those discussed above may be required to enable definition of a graphical path. In this regard, it should be noted that various other embodiments are not limited to the details of these embodiments, which are referred to for purposes of illustration only. In some embodiments in which the encircling path is drawn, selected, or otherwise indicated in a continuous manner, e.g., through motion-based user input or otherwise, the corresponding displayed path 530 may be displayed in a continuous form, instead of the discontinuous form shown in FIG. 5J.

It is noted that the definition or indication of the encircling path in accordance with the instructions associated with block 604 (and block 604b, block 604c, and some embodiments of block 604a, or a combination thereof in some embodiments) of FIG. 6A is not limited to embodiments involving graphical representations including two-dimensionally depicted graphical entities (e.g., FIG. 5J), and may be associated with embodiments involving a graphical representation 500 including three-dimensionally depicted entities. In this regard, in some embodiments, a user may indicate the encircling path with reference to a three-dimensional reference. In some embodiments, a user may draw, select, or otherwise indicate the encircling path in or among a three-dimensional representation. For example, FIG. 5K shows the displayed path 530 as described with respect to FIG. 5J but generated on a three-dimensionally depicted portion of the graphical representation 500. In some embodiments, the encircling path defines at least three or at least five non-colinear locations in the graphical representation 500. According to various embodiments, the non-colinear locations may be depictable two-dimensionally in graphical space or three-dimensionally in graphical space.

In some embodiments, the encircling path indicated by the user input need not be displayed. For example, the encircling path indicated by the user input could be utilized, e.g., by the data processing device system, to select one or more transducers for activation that are inside or outside of the encircling path or to determine a region of interest in the graphical representation 500 or in real-world space corresponding to a region of interest in the graphical representation 500, thereby a need to visually present the encircling path itself may not be desirable or needed, since the item(s) of interest may be inside or outside of such encircling path, in some embodiments. In other words, the encircling path itself may not be of particular interest as much as items in a region of space inside or outside of the encircling path, according to some embodiments. In some embodiments, the encircling path indicated by the user input need not be displayed in the graphical representation 500 at any time or at the time that the user input is generated by the user. In some embodiments, the encircling path indicated by the user input is not displayed in the graphical representation 500 in response to the user input.

In some embodiments, more than one path may be indicated by the user input. For example, according to some embodiments associated with block 604c, at least two encircling paths may be provided, with one encircling path nested in another encircling path. For example, FIG. 5O, described in more detail below, includes a first encircling path 529a and a second encircling path 529b. The first encircling path 529a may be, e.g., according to block 604b2 among a first group of graphical elements 530a (three called out in FIG. 5O) illustrated with an internal horizontal line pattern in FIG. 5O. The second encircling path 529b may be, e.g., according to block 604c among a second group of graphical elements 530b (two called out in FIG. 5O) illustrated with an internal vertical line pattern in FIG. 5O. In some embodiments associated with block 604cl, at least a portion of the second encircling path 529b may be located interior of the first encircling path 529a. Embodiments having more than one path may be particularly beneficial at least in some contexts where it is desired to identify a region of interest between the paths. With reference to the example of FIG. 50, the region of interest may be a region between the first encircling path 529a and the second encircling path 529b.

Referring to FIG. 6B, determination instructions are associated with block 606, where the data processing device system 110, 310 may be configured by such instructions, according to some embodiments, to determine a first region of space. In some embodiments where the user input from block 604, 604b, or 604c indicates an encircling path, the first region of space may be in a determined positional relationship with respect to the encircling path per block 606a. In some other embodiments in which the user input does not necessarily define or indicate an encircling path, such as per some embodiments of blocks 604 or 604a, but identifies a selected graphical element set associated with an arrangement of transducers distributed around a particular region of space, the first region of space may be in a determined positional relationship with respect to the arrangement of transducers per block 606b. In some embodiments where the user input from block 604, 604b, 604b1, 604b2, or 604c indicates multiple encircling paths, the first region of space may be a region between the encircling paths per block 606c. For example, in some of these embodiments, the first region of space may be interior of the first encircling path 529a in FIG. 5O and may be exterior of the second encircling path 529b in FIG. 5O.

In some embodiments, the first region of space determined according to program instructions associated with block 606 is a region of interest. In some embodiments in which the user input from block 604 defines or indicates an encircling path, the determined positional relationship may be interior of the encircling path. In some embodiments, the determined positional relationship may be exterior of the encircling path. In some embodiments, the determined positional relationship is not both interior and exterior of the encircling path. For example, in each of FIGS. 5J and 5K, a first region of space 540 is determined to be in a positional relationship that is interior of the displayed path 530 which corresponds to or represents the indicated encircling path, according to some embodiments. FIG. 50 illustrates some embodiments in which the first region of space 540 is determined to be in a positional relationship that is exterior of the encircling path 529b. At least some of such embodiments may be particularly useful for, among other contexts, defining a region of space between two boundaries, such as between two encircling paths 529a, 529b in the example of FIG. 5O. However, at least some of such embodiments may be particularly useful in other contexts as well. For instance, if one or more encircling paths, one or more displayed paths 530, or one or more arrangements of transducers (identified per block 604a) are intended to identify one or more exclusionary regions where, e.g., ablation is to be avoided, the first region of space may be determined as outside or exterior of the one or more encircling paths, one or more displayed paths 530, or one or more arrangements of transducers. For example, FIG. 5P illustrates an embodiment in which the encircling path 529c (which may or may not be graphically presented) indicates an exclusionary region 546 that is not to be, e.g., ablated, such that the first region of space 540 is determined as being exterior or outside of the encircling path 529c. Such a circumstance may result in an activation for tissue ablation of the transducers associated with transducer graphical elements 502 shown with a horizontal line internal pattern in FIG. 5P, according to some embodiments.

Determination of whether a particular region in the graphical representation 500 is within the encircling path or exterior of the encircling path may include determination of display coordinates (e.g., pixel coordinates) of various particular points that are within or outside the boundary formed by the encircling path. Such a determination may be made, e.g., at least by following the manner in which a graphical painting or art program, known in the art, fill in a bounded graphical region with a paint color fill function. That is, in such a graphical painting or art program, when a user selects a graphical location for the program to fill in a region that contains such graphical location with a user-selected color, the graphical painting or art program identifies a graphical region around the user-selected graphical location that has the same or similar pixel colors as the user-selected graphical location. The boundary of the graphical region to be color-filled is detected as having a different or substantially different pixel color as the user-selected graphical location. With the graphical region defined, the program fills in such region with the user-selected color. It may be considered a type of graphical masking operation. Similarly, with the encircling path defined (or a corresponding displayed path 530 defined according to some embodiments), such path may be utilized as such a boundary, possibly with a unique pixel color value set for such path, so that a region within or outside of such path may be determined to be the first region of space associated with block 606, according to some embodiments. Of course, other manners of identifying or determining the first region of space may be used in other embodiments.

In some embodiments in which the user input from block 604 indicates a selected graphical element set associated with an arrangement of transducers distributed around a particular region of space as per block 604a, the first region of space may be in a determined positional relationship (e.g., interior, exterior, or both in some embodiments) with respect to the arrangement of transducers. Such a first region of space in a determined positional relationship with respect to the arrangement of transducers may be determined in a similar manner as discussed above with respect to the encircling path, where the arrangement of transducers may be modeled in computer memory (e.g., memory device system 130, 330) to determine a boundary (which may be an encircling path in some embodiments) that passes through the arrangement of transducers, such boundary facilitating definition of the first region of space. If, for example, the desired positional relationship is interior of the arrangement of transducers, the first region of space may be defined as being a region within such boundary, according to some embodiments. If, for example, the desired positional relationship is exterior of the arrangement of transducers, the first region of space may be defined as being a region outside such boundary, according to some embodiments.

In some embodiments, graphical representation instructions, such as those associated with block 602 or otherwise, may configure a data processing device system 110, 310 to change a visual characteristic set of a particular region of the graphical representation 500 interior or exterior of the encircling path in response to a determination that the particular region interior or exterior, respectively, of the encircling path or arrangement of transducers is the first region of space. In some embodiments, the determined positional relationship indicating whether the first region of space is a particular region of space that is interior an encircling path or arrangement of transducers or is a particular region of space that is exterior an encircling path or arrangement of transducers is a predetermined positional relationship (e.g., a determination made prior to reception of the user input indicating the encircling path (e.g., per block 604b or block 604c) or graphical element set associated with the arrangement of transducers (e.g., per block 604a) by a user, with data indicating the predetermined positional relationship being stored in the memory device system 130, 330 and being accessed by the data processing device system 110, 310 for the determination of the first region of space. In some embodiments, the determined positional relationship indicating whether the first region of space is a particular region of space that is interior an encircling path or arrangement of transducers or is a particular region of space that is exterior an encircling path or arrangement of transducers is made in response to user input indicating a particular selection. In some embodiments, the graphical representation instructions associated with block 602 or otherwise may allow the user to toggle between a first particular region of the graphical representation 500 interior of the encircling path or arrangement of transducers and a second particular region of the graphical representation 500 exterior of the encircling path or arrangement of transducers to, for example, assist the user in making a selection of the first particular region or the second particular region as the first region of space. In some embodiments, (a) a visual characteristic set of the first particular region of space, (b) a visual characteristic set of the second particular region of space, or both (a) and (b) may change in response to toggling between the first particular region of space and the second particular region of space.

In some embodiments in which the first region of space is determined with respect to the encircling path (which may be represented by displayed path 530 in some embodiments) or arrangement of transducers, the first region of space may be determined to include or exclude the encircling path or the displayed path 530 or the arrangement of transducers. In other words, in various embodiments, the first region of space may be determined to itself include or exclude the encircling path or the displayed path 530 or the arrangement of transducers, according to various embodiments. In some embodiments, the first region of space may be determined to be some, but not all, of the portion(s) of the graphical representation 500 located interior or exterior, depending on the embodiment, of the encircling path, the displayed path 530, or the arrangement of transducers.

In some embodiments, in which the first region of space may be determined to be some, but not all, of the portions of the graphical representation 500 located interior or exterior, depending on the embodiment, of the encircling path, displayed path 530, or the arrangement of transducers, the determination may be made on the basis of transducer data. Such transducer data may be received, via input-output device system 120, 320, according to program instructions associated with block 607. In this regard, although block 607 is shown in FIG. 6B as being after block 606 it may also exist before or before and after block 606 in some embodiments. In some embodiments, block 607 is excluded. Transducer data such as transducer-to-tissue contact data (e.g., described above in this disclosure) may be received by the data processing device system 110, 310, indicating varying degrees of transducer-to-tissue contact, and particular regions of the graphical representation 500 located interior or exterior, depending on the embodiment, of the encircling path, displayed path 530, or the arrangement of transducers and associated with a particular level transducer-to-tissue contact may or may not be included in the first region of space.

In some embodiments, the determined positional relationship indicating whether the first region of space (a) is a particular region of space that is interior an encircling path, displayed path 530, or the arrangement of transducers, or (b) is a particular region of space that is exterior an encircling path, displayed path 530, or the arrangement of transducers, may be made, at least in part, on the basis of transducer data, e.g., which may be received per block 607, as discussed above. In some embodiments, the encircling path, displayed, path, or arrangement of transducers may encircle a region corresponding to a bodily opening (e.g., a pulmonary vein). Transducer data such as transducer-to-tissue contact data may indicate a lesser degree of tissue contact interior the encircling path as compared to a degree of tissue contact determined exterior the encircling path, and the determined positional relationship may be made on a comparison of differences in the determined degrees of tissue contact. In a similar manner, transducer data indicating a greater degree of fluid flow (for example, as described above in this disclosure) in a region corresponding to being interior the encircling path, displayed path 530, or the arrangement of transducers (e.g., when a bodily opening such as a pulmonary vein is encircled) may be used to determine the positional relationship.

In some embodiments, the determination of the first region of space according to program instructions associated with block 606 need not be determined solely with respect to the encircling path. In this regard, in some embodiments, the first region of space may additionally or alternatively be determined with respect to the arrangement of transducers, e.g., associated with the selected graphical element set per block 604a in some embodiments. In this regard, in some embodiments associated with block 606b, the determined positional relationship utilized to determine the first region of space may be interior or exterior of the arrangement of transducers or their corresponding graphical elements selected per block 604a. In the example of FIG. 5J, the data processing device system 110, 310 may be configured by program instructions associated with block 606b to determine the first region of space as being in a determined positional relationship (e.g., interior or exterior) with respect to the arrangement of transducers associated with the graphical elements along or adjacent displayed path 530. In this regard, the first region of space determined according to block 606 may exist in real-world three dimensional space where the physical transducers (e.g., 220, 306) exist, e.g., in the case of block 606b, or the first region of space may exist in graphical space (which may be one, two, or three dimensional depending on the graphical representation) inside the memory device system 130, 330 that formulates the graphical representation (e.g., graphical representation 500), e.g., in the case of at least block 606a, according to various embodiments. In some embodiments in which the first region of space exists in real-world space, the data processing device system 110, 310 may still be configured to determine such first region of space based on calculations that exist within its memory device system 110, 310, but such calculations result in a determination of a region of space that describes, models, or otherwise represents real-world space, e.g., as opposed to a region of the graphical representation 500 which resides in graphical space.

In some embodiments where the user input from block 604, 604b, or 604b1 indicates multiple encircling paths, the first region of space may be a region between the encircling paths per block 606c. For example, in some of these embodiments, the first region of space may be interior of the first encircling path 529a in FIG. 5O and may be exterior of the second encircling path 529b in FIG. 5O.

Referring to FIG. 6B, selection instructions are associated with block 608. In some embodiments, the selection instructions associated with block 608 are configured to cause, in response to reception of the user input (e.g., per various embodiments of block 604) indicating the encircling path or arrangement of transducers and in response to the determination (e.g., per various embodiments of block 606) of the first region of space, a machine-based selection of a transducer set (which may be referred to as a first transducer set) of the plurality of transducers (e.g., 220, 306, according to some embodiments). According to some embodiments, the machine-based selection may select (e.g., according to program instructions associated with at least block 608 or block 608a) each transducer in the first transducer set as having at least part of a corresponding graphical element of the displayed plurality of graphical elements 501 in the determined first region of space. In some embodiments in which the determined first region of space (e.g., per at least block 606b) is in real-world space (e.g., as opposed to graphical space), the machine-based selection may select (e.g., according to program instructions associated with at least block 608 or block 608b) each transducer in the first transducer set as being in, at least in part in some embodiments, the determined first region of space. According to some embodiments, the selected first transducer set includes some, but not all, of the transducers in the plurality of transducers.

In FIG. 6B, activation instructions are associated with block 610, and the activation instructions configure the data processing device system 110, 310 in some embodiments to cause, via the input-output device system 120, 320, activation of selected transducers of the plurality of transducers including each transducer in the first transducer set machine-selected per block 608. In some embodiments associated with block 604a in which the user input indicates a selected graphical element set associated with an arrangement of transducers, the activation according to block 610 may include, per block 610a, each transducer in the arrangement of transducers and each transducer in the first transducer set. In this regard, in some embodiments, the activation according to block 610a may activate selected transducers including, not only the one or more machine-selected transducers selected according to some embodiments of block 608, but also one or more additional transducers that, in some embodiments are user-selected according to some embodiments associated with block 604a by the user selection of the graphical element set that corresponds to such one or more additional transducers. Accordingly, in some embodiments, for example, a user may select an arrangement of transducers and then, according to some embodiments of block 608, a machine-based selection may additionally select one or more additional transducers inside or outside, depending on the embodiment, of that arrangement of transducers, such that both the user-selected arrangement of transducers and the machine-selected transducers are activated according to program instructions associated with block 610a, in some embodiments. According to some embodiments, the activation according to block 610, block 610a, or both, (e.g., of each transducer in the first transducer set and, in the case of block 610a each transducer in the arrangement of transducers) is configured to cause tissue ablation. According to some embodiments, the activation according to block 610, block 610a, or both is configured to cause thermal ablation of tissue. According to some embodiments, the activation according to block 610, block 610a, or both is configured to cause pulsed field ablation of tissue. In some embodiments, the activation per block 610, block 610a, or both) of the selected transducers (which may include each transducer in the arrangement of transducers and each transducer in the first transducer set at least in the case of block 610a) is configured to cause an ablated tissue lesion that at least partially surrounds at least part of the first region of space 540, as would be the case upon activation of the selected transducers at least in the example of FIGS. 5N and 5Q, in some embodiments.

According to some embodiments, the selection instructions associated with block 608, block 604a, or both, are configured to cause a selection indicating a selected graphical element set from the displayed plurality of graphical elements 501, the selected graphical element set corresponding to the first transducer set of the plurality of transducers, the arrangement of transducers, or both the first transducer set and the arrangement of transducers. According to some embodiments, a visual characteristic set of the graphical elements of the selected graphical element set may change in response to the selection(s) indicating the selected graphical element set. FIGS. 5L and 5M respectively show the two-dimensional and three-dimensional graphical representations of FIGS. 5J and 5K with a selected graphical element set 545 (including an outer ring 545a (four elements called out in FIG. 5M) and an inner ring 545b (three elements called out in FIG. 5M) of selected graphical elements) selected in accordance with the selection instructions associated with block 608, block 604a, or both, according to some various embodiments. In FIGS. 5L and 5M, selected graphical elements 502 in the selected graphical element set 545 are indicated with an internal line pattern fill to distinguish them from unselected graphical elements 502, which are shown as not having an internal pattern fill, according to some embodiments.

According to some embodiments, the machine-based selection according to at least block 608 (or block 608a or 608b in some embodiments thereof) may include a first machine-based selection of a first graphical element of a selected graphical element set, the selected graphical element set corresponding to the first transducer set, and the first graphical element corresponding to a first transducer in the first transducer set. In some embodiments in which the user input associated with block 604 indicates an encircling path, the first graphical element may be selected according to the first machine-based selection as a first particular one of the plurality of graphical elements located closest to a portion of the encircling path among all graphical elements of the plurality of graphical elements located at least in part in the first region of space. For example, in FIGS. 5L and 5M, the first graphical element 502I1 is closest among all of the transducer graphical elements 502 in the first region of space 540 to a portion 530-a1 of the encircling path (corresponding to displayed path 530 in some embodiments). According to some embodiments, the portion of the encircling path is a particular location or point along the encircling path.

It should be noted that, while some discussions herein pertain to graphical elements and their characteristics and relationships therebetween, such discussions correspondingly apply to the transducers associated with such graphical elements, in some embodiments. For example, according to some embodiments, the machine-based selection per block 608 may include a first machine-based selection of a first transducer in the first transducer set, the first transducer selected according to the first machine-based selection as being closest to a particular transducer in the arrangement of transducers (e.g., associated with the graphical element set indicated by the user input per at least block 604a) among all transducers of the plurality of transducers that are not in the arrangement of transducers and are located at least in part in the first region of space. For instance, in FIGS. 5L and 5M, the user-input received per block 604 may result in displayed path 530, and the transducer graphical elements that intersect the path 530 may be deemed selected graphical elements associated with an arrangement of transducers per block 604a, in some embodiments. In this regard, for example, the machine-selection per some embodiments of block 608 may include a first machine-selection of a first transducer associated with transducer graphical element 502H2 as being closest to the particular transducer associated with transducer graphical element 502H1 in the arrangement of transducers (intersected by displayed path 530 in this example), where the transducer associated with transducer graphical element 502H2 is closest to the particular transducer associated with transducer graphical element 502H1 in the arrangement of transducers among all transducers of the plurality of transducers that are not in the arrangement of transducers and are located at least in part in the first region of space (e.g., the real world space corresponding to first region of space 540 in FIGS. 5L and 5M), in some embodiments.

In some embodiments, the machine-based selection according to at least block 608 (or block 608a or block 608b in some embodiments thereof) may include a second machine-based selection of a second graphical element of the selected graphical element set corresponding to the first transducer set. According to some embodiments in which the user input associated with block 604 indicates an encircling path, the second graphical element may be selected according to the second machine-based selection as a second particular one of the plurality of graphical elements located closest, besides the first graphical element, to the portion of the encircling path, among all graphical elements of the plurality of graphical elements that are in the first region of space and spaced from the encircling path. According to some embodiments, each particular graphical element of the plurality of graphical elements that is located in the first region of space and that is adjacent the encircling path has no other graphical element of the plurality of graphical elements located in the first region of space located between the particular graphical element and the encircling path. In FIG. 5L, a second transducer graphical element 50212 is a second particular one of the plurality of transducer graphical elements 502 located closest, besides the first graphical element 502I1, to the portion of the encircling path (represented by portion 530-a1 of the displayed path 530), among all of the transducer graphical elements 502 of the plurality of transducer graphical elements 502 that are in the first region of space 540 and are spaced from the encircling path. In FIG. 5M, however, due to the three-dimensional perspective, a second transducer graphical element 502J1 is a second particular one of the plurality of transducer graphical elements 502 located closest, besides the first graphical element 502I1, to the portion of the encircling path (represented by portion 530-a1 of the displayed path 530), among all of the transducer graphical elements 502 of the plurality of transducer graphical elements 502 that are in the first region of space 540 and are spaced from the encircling path. Accordingly, it can be seen that, in some various embodiments, the second graphical element may be selected according to the second machine-based selection utilizing distances in a two-dimensional projection representation (e.g., as in FIG. 5L) or in a three-dimensional representation (e.g., as in FIG. 5M). In some embodiments, the second graphical element is spaced from the encircling path. In some embodiments, the second graphical element corresponds to a second transducer in the machine-selected (e.g., per block 608, 608a, or 608b in various embodiments) first transducer set.

According to some embodiments in which the user input per block 604 indicates an encircling path, the first graphical element (e.g., first graphical element 502I1 in some embodiments) selected according to the first machine-based selection may be located at least in part between the second graphical element (e.g., second graphical element 50212 in some embodiments such as with respect to FIG. 5L) selected according to the second machine-based selection and the portion of the encircling path (e.g., represented by portion 530-a1 of the displayed path 530). In some embodiments, the first graphical element (e.g., first graphical element 502I1 in some embodiments) selected according to the first machine-based selection and the second graphical element (e.g., second graphical element 50212 in some embodiments such as with respect to FIG. 5L) selected according to the second machine-based selection are adjacent graphical elements. In some embodiments, the first transducer (e.g., corresponding to the first graphical element) in the first transducer set and the second transducer (e.g., corresponding to the second graphical element) in the first transducer set are adjacent transducers. In this regard, in some embodiments, the machine-based selection per block 608 may be considered to include a selection of at least a first transducer in the first transducer set as adjacent a particular transducer of the arrangement of transducers associated with the first transducer set. According to various embodiments, the first graphical element selected according to the first machine-based selection is spaced from the encircling path. For example, in FIGS. 5L and 5M, transducer graphical element 502I1 selected as the first graphical element is spaced (albeit by a small amount in FIGS. 5L and 5M) from the encircling path (represented by displayed path 530 according to some embodiments). In some embodiments, the first graphical element selected according to the first machine-based selection is intersected by the portion of the encircling path. For example, in FIGS. 5L and 5M, if the transducer graphical element 502L1 is selected as the first graphical element to be selected in accordance with the first machine-based selection (e.g., per at least block 608, or block 608a, or block 608b in some embodiments), such graphical element is intersected by a portion of the encircling path (represented by displayed path 530 according to some embodiments). FIGS. 5S-1 and FIGS. 5S-2, discussed in further detail below, provide examples and discussions of when a graphical element set and corresponding transducer set may be considered machine-selected when intersected by a user-produced encircling path, according to some embodiments.

In some embodiments, the machine-based selection (e.g., per at least block 608, 608a, or 608b in some embodiments) includes a first machine-based selection of a first group of graphical elements of the plurality of graphical elements, each graphical element in the first group of graphical elements of the plurality of graphical elements corresponding to a respective transducer in a first group of transducers in the first transducer set. For example, in some embodiments associated with block 608, 608a, or 608b, the data processing device system 110, 310 may be configured to select the transducer set by way of first selecting a first group of graphical elements corresponding to such transducer set. According to some embodiments in which the user input per block 604 indicates an encircling path, each graphical element in the first group of graphical elements of the plurality of graphical elements is selected according to the first machine-based selection as a particular one of the plurality of graphical elements located in the first region of space closest to a respective portion of the encircling path among all graphical elements of the plurality of graphical elements located in or located at least in part in, depending on the embodiment, the first region of space. For example, in FIGS. 5L and 5M, the first group of transducer graphical elements 502 selected according to the first-machine-based selection instructions may include some or all of the graphical elements 502 in the graphical element line between the graphical elements 502F1 and 502L1, inclusive, the graphical elements 502 in the graphical element line between the graphical elements 502L1 and 502L5, inclusive, the graphical elements 502 in the graphical element line between the graphical elements 502L5 and 502F5, inclusive, and the graphical elements 502 in the graphical element line between the graphical elements 502F5 and 502F1, inclusive, according to some embodiments, since those graphical elements are in the first region of space 540 and are closest to their respective portions of the encircling path (corresponding to displayed path 530 in the examples of FIGS. 5L and 5M). In this example, each of the transducer graphical elements 502 in the first group of transducer graphical elements 502 is located closest to a respective portion of the encircling path (e.g., represented by the displayed path 530 according to some embodiments) among all transducer graphical elements 502 of the plurality of transducer graphical elements 502 located in the first region of space 540. According to various embodiments, no other transducer graphical element 502 in the first region of space 540 is located between each transducer graphical element 502 in the first group of transducer graphical elements 502 and the respective portion of the encircling path.

In this regard, “respective portion” in this context is intended in some embodiments to refer to the portion of the encircling path closest to the respective graphical element 502, with no other graphical element 502 within the first region of space 540 closer to such respective portion of the encircling path. For instance, for graphical element 502F1, its respective portion of the encircling path may be represented by portion 530-a2 of displayed path 530. Since such portion 530-a2 intersects/overlaps graphical element 502F1, graphical element 502F1 is closest to that portion 530-a2 compared to all other graphical elements 502 in the first region of space 540. For another instance, for graphical element 502I1 (which is in the graphical element line between the graphical elements 502F1 and 502L1), the respective portion of the encircling path may be represented by portion 530-a1 of displayed path 530, since such portion 530-a1 is closest to the graphical element 502I1 and no other graphical element 502 in the first region of space 540 is closer to such portion 530-a1.

In some embodiments in which the user input per block 604 indicates an encircling path, the first group of graphical elements of the plurality of graphical elements selected according to the first machine-based selection includes each graphical element of the plurality of graphical elements that is in or is at least in part in, depending on the embodiment, the first region of space and is located closest to a respective portion of the encircling path among all graphical elements of the plurality of graphical elements located in or located at least in part in, depending on the embodiment, the first region of space. In this regard, in some embodiments, no other graphical element of the plurality of graphical elements in the first region of space is located between each graphical element in the first group of graphical elements and the respective portion of the encircling path. As described above, with respect to FIGS. 5L and 5M, for example, the first group of transducer graphical elements 502 may include every particular transducer graphical element 502 in the first region of space 540 that is closest to a respective portion of the encircling path (e.g., represented by the displayed path 530, according to some embodiments) among all of the transducer graphical elements 502 in the first region of space 540.

According to some embodiments, the machine-based selection (e.g., per at least block 608, 608a, or 608b in some embodiments) includes a second machine-based selection of a second group of graphical elements of the plurality of graphical elements. According to some embodiments, each graphical element in the second group of graphical elements of the plurality of graphical elements is selected according to the second machine-based selection as a particular one of the plurality of graphical elements in the first region of space 540 other than any graphical element in the first group of graphical elements of the plurality of graphical elements. According to some embodiments, each graphical element in the second group of graphical elements of the plurality of graphical elements corresponds to a respective transducer in a second group of transducers in the first transducer set. According to some embodiments in which the user input per block 604 indicates an encircling path, each graphical element in the second group of graphical elements of the plurality of graphical elements does not include any graphical element of the plurality of graphical elements located in the first region of space that is closest to a respective portion of the encircling path among all of the plurality of graphical elements located in the first region of space. For example, in FIGS. 5L and 5M, the second group of transducer graphical elements selected according to the second machine-based selection instructions may include some or all of at least transducer graphical elements 502K2, 502K3, 502K4, 502 J4, 50214, 502H4, 502G4, 502G3, 502 G2, 502H2, 50212, and 502J2 located in the first region of space 540, according to some embodiments. According to various embodiments, each of the transducer graphical elements in the second group of transducer graphical elements 502 selected according to the second machine-based selection does not include any transducer graphical element 502 located in the first region of space 540 that is closest to a respective portion of the encircling path (e.g., represented by displayed path 530 according to some embodiments) among all of the plurality of transducer graphical elements 502 located in the first region of space 540. In this regard, in some embodiments, the first group of graphical elements may represent the outermost transducer graphical elements within the encircling path represented by displayed path 530, and the second group of graphical elements may represent transducer graphical elements interior of the first group of graphical elements, in some embodiments. Such groupings of graphical elements may efficiently allow the data processing device system 110, 310 to select graphical elements throughout the first region of space 540, e.g., by first identifying an outer encircling set of graphical elements and then select other graphical elements interior of that outer encircling set, according to some embodiments.

In some embodiments, the second group of graphical elements selected according to the second machine-based selection is a group of some, but not all, of the graphical elements in the first region of space but excluding the graphical elements in the first group of graphical elements selected according to the first machine-based selection. For example, in FIGS. 5L and 5M, while some embodiments may select graphical elements 502K2, 502K3, 502 K4, 502J4, 50214, 502H4, 502G4, 502G3, 502 G2, 502H2, 50212, and 502J2 as the second group of graphical elements selected according to the second machine-based selection, other embodiments may, e.g., select fewer than all of these graphical elements or may, e.g., select or additionally select other graphical elements (e.g., some or all of graphical elements 502H3, 50213, 502J3, in some embodiments) as the second group of graphical elements. In some embodiments, a group of the graphical elements of the plurality of graphical elements located at least in part in the first region of space and selected according to the first machine-based selection and the graphical elements of the plurality of graphical elements located at least in part in the first region of space and selected according to the second machine-based selection is group of some, but not all, of the graphical elements of the plurality of graphical elements located at least in part in the first region of space. For instance, due to anatomical feature characteristics in the first region of space 540 or due to ablative energy management, or some other need or parameter, it may be desirable to select fewer than all available graphical elements 502 in the first region of space 540. In some embodiments, the second group of graphical elements selected according to the second machine-based selection is a group of all of the graphical elements in the first region of space but excluding the graphical elements in the first group of graphical elements selected according to the first machine-based selection. In some embodiments, a group of the graphical elements of the plurality of graphical elements located at least in part in the first region of space and selected according to the first machine-based selection and the graphical elements of the plurality of graphical elements located at least in part in the first region of space and selected according to the second machine-based selection is a group of all of the graphical elements of the plurality of graphical elements located at least in part in the first region of space. For instance, in some embodiments, the machine-selected first and second groups of graphical elements 502 may be all of the graphical elements 502 in or at least in part in the first region of space 540. At least some of such embodiments may be beneficial at least to provide an efficient mechanism for selecting all transducer graphical elements in the first region of space 540 for activation.

In some embodiments, each graphical element in the second group of graphical elements of the plurality of graphical elements selected according to the second machine-based selection is adjacent each of at least one graphical element in the first group of graphical elements of the plurality of graphical elements selected according to the first machine-based selection. For example, in FIGS. 5L and 5M, each of transducer graphical elements 502H2 and 50212, which may be included in the second group of transducer graphical elements, is adjacent transducer graphical elements 502H1 and 502I1, which may be included in the first group of transducer graphical elements, according to some embodiments. According to some embodiments, each transducer in the second group of transducers is adjacent each of at least one other transducer in the second group of transducers, as is the case, for example, with respect to adjacent transducer graphical elements 502H2 and 50212 in FIGS. 5L and 5M, which may be included in the second group of transducers, in some embodiments.

In some embodiments, (a) the first group of graphical elements of the plurality of graphical elements selected according to the first machine-based selection, (b) the second group of graphical elements of the plurality of graphical elements selected according to the second machine-based selection, or each of (a) and (b) are arranged in a ring-like or encircling spatial distribution in the graphical representation. For instance, in the example of FIGS. 5L and 5M, the first group of transducer-graphical elements are arranged in a ring-like or encircling spatial distribution, such first group including, in some embodiments, the graphical elements 502 in the ring spanning from graphical element 502F1 to graphical element 502L1 to graphical element 502L5 to graphical element 502F5 and back to graphical element 502F1. Other embodiments may have the second group of graphical elements in a ring-like or encircling spatial distribution, such as the ring of graphical elements spanning from graphical element 502G2 to graphical element 502K2 to graphical element 502K4 to graphical element 502G4 and back to graphical element 502G2, in some embodiments. In some embodiments in which both the first group and second group of graphical elements are in a ring-like or encircling spatial distribution, such a configuration may be beneficial to, among other things, provide a thick (e.g., two-transducer wide) ablation path encircling an anatomical structure, e.g., when treating atrial fibrillation. Such a thick path may provide a more suitable electrophysiological conduction block compared to a single-transducer wide path. However, in other contexts, such a thick path may not be desirable or needed.

In some embodiments in which the user input per block 604 indicates an encircling path, each ring-like or encircling spatial distribution of the graphical elements defines a shape that is similar to a shape of the first region of space defined at least in part by the encircling path (which may be represented by the displayed path 530 in some embodiments). For instance, the first group of graphical elements in the above-discussed examples associated with FIGS. 5L and 5M has the same or similar shape as the encircling path but is nested within it. In this regard, the first group of graphical elements in the above-discussed examples associated with FIGS. 5L and 5M outlines the first region of space 540 and, therefore, may be considered to have a same or similar shape as the first region of space 540.

However, other embodiments do not have such a feature. For example, in some embodiments, each ring-like or encircling spatial distribution of the graphical elements defines a shape that is different than a shape of the first region of space defined at least in part by the encircling path. For example, depending on desired transducer activation or tissue treatment parameters, different sets of graphical elements and associated transducers may be machine-selected according to program instructions associated with block 608 (or block 608a or 608b in some embodiments thereof), thereby causing different shapes of selected graphical elements and associated transducers, according to various embodiments.

According to some embodiments, a ring-like or encircling spatial distribution of the graphical elements selected according to the first machine-based selection and a ring-like or encircling spatial distribution of the graphical elements selected according to the second machine-based selection combine to form a particular ring-like or encircling spatial band of the graphical elements in the first region of space. In some embodiments, the particular ring-like or encircling band of the graphical elements may have a width of at least two graphical elements around the particular ring-like or encircling band. According to various embodiments, the spatial distribution of graphical elements defined by the machine-based selection may include a band of the graphical elements that extends along a path in the first region of space, the band of graphical elements having a width of at least two graphical elements (e.g., or associated transducers) wide as the path is traversed. For example, the outer portion of the band (having a single graphical element width in some embodiments) may include the graphical elements selected according to the above-discussed first machine-based selection, and the inner portion of the band (having a single graphical element width in some embodiments) may include the graphical elements selected according to the above-discussed second machine-based selection, according to some embodiments. Or, in some embodiments, the inner portion of the band (having a single graphical element width in some embodiments) may include the graphical elements selected according to the above-discussed first machine-based selection, and the outer portion of the band (having a single graphical element width in some embodiments) may include the graphical elements selected according to the above-discussed second machine-based selection, according to some embodiments. In some embodiments, a portion of the band may be considered to be at least in part user-selected, e.g., by the user defining an encircling path that intersects one or more transducer graphical elements 502 in a portion of the band, such intersection indicating at least in part a user-selection of such intersected transducer graphical elements, and another portion of the band (e.g., interior or exterior of the encircling path, but not intersecting the encircling path) may be considered machine-selected, in some embodiments.

In FIG. 5N, a simple grid of transducer graphical elements 502 is represented as squares for case of illustration. In this regard, FIG. 5N (as well as each of the figures between FIG. 5N and FIG. 5S-2, inclusive) may be considered a simplified version of the graphical representations 500 shown in FIGS. 5A-5M, in some embodiments, e.g., for case of illustration. Only one graphical element 502 is called out in FIG. 5N for simplicity, but each square in FIG. 5N is a graphical element 502 in this example. The encircling path produced according to user input per block 604, block 604b, or block 604c is represented by a hand-drawn path 529 in FIG. 5N. Such hand-drawn path 529 may or may not be displayed by a display device system (e.g., display device system 332 in some embodiments) depending on the embodiment, and, therefore, may not be visible to the user. However, the path 529 is shown in FIG. 5N at least for illustration and understanding purposes. In the example of FIG. 5N, the displayed path 530, which may be an optional displayed representation of the encircling path, is indicated or defined by the squares with a horizontal interior line pattern. In the example of FIG. 5N, the outer portion of the band of graphical elements selected according to the above-discussed first machine-based selection is represented by the graphical elements 532 (three called out in FIG. 5N as 532, and two called out in FIG. 5N as 532a, 532b) with the internal pattern having lines starting toward the upper right and ending toward the lower left of the respective square. The inner portion of the band of graphical elements selected according to the above-discussed second machine-based selection is represented by the graphical elements 534 (three called out in FIG. 5N as 534, and two called out in FIG. 5N as 534a, 543b) with the internal pattern having lines starting toward the upper left and ending toward the lower right of the respective square. According to some embodiments, the path (e.g., represented by the outer ring of graphical elements 532 and the inner ring of graphical elements 534 in the example of FIG. 5N) extends along a closed path in the first region of space 540. According to some embodiments, the path extends around a portion of the first region of space that includes at least a particular set of graphical elements of the plurality of graphical elements located in the first region of space, the particular set of graphical elements of the plurality of graphical elements not selected according to the machine-based selection. According to some embodiments, each graphical element in the particular set of graphical elements (e.g., inside the inner ring, in some embodiments) is unselected. For instance, in some embodiments, such as that shown in FIG. 5N, where the first machine-based selection provides an outer ring of selected graphical elements 532, and the second machine-based selection provides an inner ring of selected graphical elements 534, there may be other graphical elements 536 within the inner ring that are not selected. In some embodiments, such unselected graphical elements (e.g., graphical elements 536 in the example of FIG. 5N) may correspond to a region of a tissue surface for which it is not desired to ablate or a region of tissue that need not be ablated. Although FIG. 5N shows outer and inner rings of graphical elements 532, 534, respectively, each having one transducer width throughout the corresponding ring for purposes of case of discussion, it should be noted that the invention is not limited to any particular transducer-ring-widths or even uniformity of width along a ring.

According to some embodiments, having the selection of graphical elements of the plurality of graphical elements include a band or block or cluster of the graphical elements of the plurality of graphical elements that includes various portions that are multiple graphical elements wide as a length of the band or block or cluster of the graphical elements of the plurality of graphical elements is traversed may be motivated for various reasons. For example, in some embodiments, the selected band or block or cluster of the graphical elements of the plurality of graphical elements may correspond to a selected band or block or cluster of the transducers in the first transducer set (e.g., per block 608 or block 608a or 608b in some embodiments thereof) that are positioned to effectively ablate a sufficiently wide region of tissue in some embodiments, to, e.g., ensure an adequate electrophysiological conduction block. In some embodiments, the selected band or block or cluster of transducers (e.g., which may be the first transducer set) may be positioned to deliver ablative energy to a contiguous region (e.g., as indicated by the selection of all graphical elements in region of space 540 in the example of at least FIG. 5M), and, in some embodiments, there may be an interior region or interior regions of tissue for which no selected transducer (e.g., as indicated by unselected graphical elements 536 in at least the example of FIG. 5N) is positioned to ablate. Such an interior region with no selected transducer, such as that indicated by unselected graphical elements 536 in at least the example of FIG. 5N, may correspond to an anatomical feature encircled by the first transducer set (e.g., indicated by selected transducer graphical elements 530, 532, and 534 in the example of FIG. 5N) for which the transfer of ablative energy is not desired or is not needed.

In some embodiments, the transducers in at least the band or block or cluster of transducers in the first transducer set are activated according to the activation instructions associated with block 610 in FIG. 6B to cause ablation (e.g., pulsed field ablation or thermal ablation, such as radiofrequency “RF” ablation) of tissue. Ablation paths created by a selection of a line of transducers that is only a single transducer in width may achieve relatively shallower ablated lesion depths. When the band or block or cluster of transducers in the first transducer set are activated to cause tissue ablation and the transducers in the band or block or cluster of transducers in the first transducer set are sufficiently close to each of other transducers (e.g., at least three other, at least four other, at least five other, depending on the desired region size) in the band or block or cluster of transducers in the first transducer set to form a contiguous lesion therebetween, the overall lesion depth will typically be greater, which may be desirable in some contexts. For example, in pulsed field ablation applications, performing the pulsed field ablation with a band, block, or cluster of electrodes will tend to enhance lesion depth by having respective lesions behave effectively as if formed by a larger electrode (with field divergence properties scaled based on the electrode size) when the electrodes are activated together as a band, block, or cluster. Accordingly, in some embodiments, enhanced efficacy may be achieved when the wider or thicker band or block or cluster of transducers in the first transducer set are activated.

According to some embodiments, a wider or thicker ablation path provides more pathways in the tissue to achieve a transmural lesion. For example, with reference to a first ring-like or encircling spatial distribution of transducers (e.g., indicated by graphical elements 532 in the example of FIG. 5N selected according to the first machine-based selection describe above), a second ring-like or encircling spatial distribution of transducers (e.g., indicated by graphical elements 534 in the example of FIG. 5N selected according to the second machine-based selection described above), and, in some embodiments, transducers (corresponding to transducer graphical elements 530 in FIG. 5N) selected as intersecting the encircling path 529, a ringed tissue area that may be resistant to achieving ablation transmurality by the activation of only a single-transducer-wide ring-like or encircling spatial distribution of transducers may, nonetheless, be ablated in a transmural manner via the ablation of more favorable tissue or a wider region of tissue by the activation of a multiple-transducer-wide ring-like or encircling spatial distribution of transducers.

Enhanced efficacy may be achieved when the band or block or cluster of transducers in the first transducer set are activated in a bipolar manner. In pulsed field ablation applications, bipolar activation of a single pair of the transducers along a “single transducer wide” band of transducers, such as represented by single pair of graphical elements 532a, 532b in the example of FIG. 5N, results in an electric field divergence that is compressed in a relatively shallow manner with respect to tissue depth in a three-dimensional (“3D”) dispersion mode. On the other hand, when adjacent transducers are activated in a bipolar activation of a “multiple transducer wide” band or block or cluster of transducers, such as represented by the addition of adjacent bipolar pair transducer graphical elements 534a, 534b to pairs 532a and 532b in the example of FIG. 5N, the electric field divergence of the resulting electric field begins to approximate a two-dimensional (“2D”) divergence, where the divergence of the electric field is not as rapid with depth into the tissue as for a more “3D” divergence form; potentially enhancing lesion depth by permitting larger voltage gradients to extend more deeply into the tissue. Further extending the number of adjacent bipolar pairs (e.g., to a 2×3 or 2×4 arrangement) will better approximate a 2D divergence pattern but with diminishing returns for potential lesion depth enhancements. Similar benefits apply to thermal ablation applications such as radiofrequency (RF) ablation, but because of the potential risk of thermal coagulum formation, selection of transducers in a “multiple transducer wide” band or block or cluster of transducers may require consideration of other factors such as which of the plurality of transducers are associated with sufficient transducer-to-tissue-contact levels. In pulsed field ablation applications, the safety aspect associated with thermal coagulum formation is not typically present.

In some embodiments, the program may include reception instructions associated with block 607 in FIG. 6B configured to cause reception, via the input-output device system 120, 320 of respective transducer data (e.g., transducer-to-tissue contact data, impedance data, fluid flow data, temperature data) for each of at least some of the plurality of transducers, and the selection instructions associated with block 608 (or block 608a or 608b in some embodiments thereof) may cause the machine-based selection of the first transducer set to be based at least in part on the received respective data for each of the at least some of the plurality of transducers. For example, in some embodiments, an inclusion of a particular transducer of the plurality of transducers as part of the first transducer set may be based on transducer-to-tissue contact data associated with the particular transducer of the plurality of transducers. In some embodiments, if the transducer-to-tissue contact data indicates that a particular level of tissue contact has not been achieved (for example, as compared to a determined or predetermined value or level) by the particular transducer of the plurality of transducers, the particular transducer may be excluded from forming part of the machine-based selection. Such exclusion may be the cause of non-selection of graphical elements 536 in the example of FIG. 5N, according to some embodiments. In some embodiments, the program may be configured to cause communication, via the input-output device system 120, 320, of various corrective actions to a user at least in response to the determination of insufficient transducer-to-tissue contact. For example, a request may be provided to the user via the input-output device system 120, 320 to reposition at least part of the transducer-based device to improve a level of transducer-to-tissue contact associated with the particular transducer of the plurality of transducers.

According to various embodiments, the machine-based selection made according to the selection instructions associated with block 608 (or block 608a or 608b in some embodiments thereof) in FIG. 6B includes a machine-based selection of a particular group of graphical elements of the plurality of graphical elements, each graphical element in the selected particular group of graphical elements of the plurality of graphical elements corresponding to a respective transducer in the first transducer set. According to some embodiments, each graphical element of at least one particular graphical element in the selected particular group of graphical elements of the plurality of graphical elements is adjacent each graphical element of a respective set of at least three graphical elements in the selected particular group of graphical elements of the plurality of graphical elements. For example, in FIGS. 5L and 5M, the selected transducer graphical element 50212 is adjacent to each graphical element in (a) a first set of three selected transducer graphical elements 502H2, 502J2, and 502I1, in some embodiments, (b) a first set of three selected transducer graphical elements identified as 502H2, 502H1, and 502I1, in some embodiments, (c) a first set of three selected transducer graphical elements 502J2, 502J1, and 502I1, in some embodiments, and (d) a first set of three selected transducer graphical elements 502H1, 502I1, and 502J1, in some embodiments. Similar relationships can be stated for each of the machine-based selection of the various other graphical elements in FIGS. 5L and 5M, and, in some embodiments, each graphical element in the selected particular group of graphical elements of the plurality of graphical elements is adjacent each graphical element of a respective set of at least three graphical elements in the selected particular group of graphical elements of the plurality of graphical elements. In some embodiments, at least one graphical element in a first respective set of at least three graphical elements in the selected particular group of graphical elements of the plurality of graphical elements is other than any graphical element in a second respective set of at least three graphical elements in the selected particular group of graphical elements of the plurality of graphical elements. For example, in FIGS. 5L and 5M, the selected transducer graphical element 50214 is adjacent each graphical element in (e) a second set of three selected transducer graphical elements 502H4, 502J4, and 50215, in some embodiments, (f) a second set of three selected transducer graphical elements identified as 502H4, 502H5, and 50215, in some embodiments, (g) a second set of three selected transducer graphical elements 502J5, 502J4, and 50215, in some embodiments, and (h) a second set of three selected transducer graphical elements 502H5, 50215, and 502J5, in some embodiments, none of which are include a graphical element that is in any the first set three transducer graphical elements associated with any of (a), (b), (c) or (d) described above. In some embodiments, each graphical element in a first respective set of at least three graphical elements in the selected particular group of graphical elements of the plurality of graphical elements is other than any graphical element in a second respective set of at least three graphical elements in the selected particular group of graphical elements of the plurality of graphical elements.

The discussion above about adjacency of graphical elements also applies to the associated transducers, in some embodiments. For instance, in some embodiments, the machine-based selection per various embodiments of block 608 may include a selection of multiple transducers in the first transducer set, each transducer in the multiple transducers in the transducer set adjacent at least one respective transducer of the arrangement of transducers. For instance, with respect to FIGS. 5L and 5M, the transducer associated with the selected transducer graphical element 50212 is adjacent to each of the transducers corresponding to selected transducer graphical elements 502H2, 502J2, and 502I1, in some embodiments.

In some embodiments in which the user input per block 604 indicates an encircling path, at least one graphical element of the at least one particular graphical element in the selected particular group of graphical elements of the plurality of graphical elements is located closest to a portion of the encircling path among all graphical elements of the plurality of graphical elements in the first region of space. For example, in FIGS. 5L and 5M, at least one particular graphical element 502I1 is located closest to a portion 530-a1 of the displayed path 530 (which may represent the encircling path) among all graphical elements 502 in the first region of space 540. In some embodiments, the selected particular group of graphical elements of the plurality of graphical elements 502 consists of some, but not all, of the graphical elements of the plurality of graphical elements in the first region of space. For example, in FIGS. 5L and 5M, the selected graphical elements shown as “hatched” with a diagonal internal line pattern do not include graphical elements 502J3, 50213, and 502H3 (such reference numerals only called out in FIG. 5M) in the first region of space 540. The same may apply to the transducers associated with the selected graphical elements. For instance, in some embodiments, selected transducers may consist of some, but not all, of the transducers located in a region of space that corresponds to the real world space represented by first region of space 540. In some embodiments, the selected particular group of graphical elements of the plurality of graphical elements includes all of the graphical elements of the plurality of graphical elements in the first region of space. For example, in some embodiments in which it is acceptable to perform ablation over an anatomical feature like region 525c in FIGS. 5L and 5M, the selected particular group of graphical elements may additionally include graphical elements 502J3, 50213, and 502H3 (such reference numerals only called out in FIG. 5M), such that all graphical elements in the first region of space 540 are selected. The same may apply to the transducers associated with the graphical elements. For instance, in some embodiments, selected transducers may include all transducers located in a region of space that corresponds to the real world space represented by first region of space 540.

According to various embodiments, each graphical element in each respective set of at least three graphical elements in the selected particular group of graphical elements of the plurality of graphical elements is adjacent to each other graphical element in the respective set of at least three graphical elements in the selected particular group of graphical elements of the plurality of graphical elements. For example, each of (b) and (c) described above is a respective set of at least three selected transducer graphical elements where each transducer graphical element 502 in the respective set of at least three selected transducer graphical elements is adjacent to each other transducer graphical element 502 in the respective set of at least three selected transducer graphical elements (e.g., in (b) transducer graphical element 502H2 is adjacent to each of transducer graphical elements 502H1 and 502I1; transducer graphical element 502H1 is adjacent to each of transducer graphical elements 502H2 and 502I1; and transducer graphical element 502I1 is adjacent to each of 502H1 and 502H2). It is noted that in each of (a) and (d) described above each transducer graphical element 502 in the respective set of at least three selected transducer graphical elements is not adjacent to each other transducer graphical element 502 in the respective set of at least three selected transducer graphical elements according to some embodiments. For example, in (a) the selected transducer graphical element 502H2 is not adjacent the selected transducer graphical element 502J2 (i.e., the selected transducer graphical element 50212 is positioned therebetween).

According to some embodiments, each graphical element of at least one particular graphical element in the selected particular group of graphical elements of the plurality of graphical elements is adjacent to each graphical element of a respective set of at least five graphical elements in the selected particular group of graphical elements of the plurality of graphical elements. For example, in FIGS. 5L and 5M, the selected transducer graphical element identified 50212 is adjacent to each graphical element in a set of five selected transducer graphical elements 502H2, 502H1, 502I1, 502J2, and 502J1.

Referring back to block 604, and particularly block 604c, according to some embodiments, the received user input may indicate not only a first encircling path, but also a second encircling path. Accordingly, in some embodiments, the data processing device system 110, 310 may be configured at least by program instructions associated with block 604c to receive user input, e.g., via the of input-output device system 120, 320, indicating a second encircling path among a second group of at least some of the displayed plurality of graphical elements (e.g., displayed transducer graphical elements 502, in some embodiments). According to some embodiments, having two encircling paths may create a nested arrangement of encircling paths, where one encircling path is located interior of the other encircling path. With such a nested arrangement of encircling paths, the first region of space determined according to program instructions associated with block 606 (and particularly block 606c, in some embodiments), may be interior of the outer encircling path and exterior of the interior encircling path, thereby, for example, allowing a user to identify a band of user-defined width of selected graphical elements and corresponding transducers for activation.

FIG. 5O provides an example, according to some embodiments. In some embodiments associated with the example of FIG. 50, the user input received according to program instructions associated with block 604 (and particularly blocks 604b, 604b2, 604c, and 604cl in some embodiments) indicates a first encircling path 529a and a second encircling path 529b. In some embodiments associated with block 604b2, the first encircling path 529a is among a first group of at least some of the displayed plurality of graphical elements 502. In the example of FIG. 5O, such first group may be the graphical elements 530a (only three called out in FIG. 5O for purposes of clarity) with the internal pattern having vertical lines. Such graphical elements 530a may be displayed with such internal pattern having vertical lines in order to highlight them graphically to a user, such highlighting being a displayed representation of the first encircling path 529a, which might not be displayed and may merely represent the path traced by the corresponding user input received according to block 604 (or, e.g., blocks 604b, 604b1, or 604b2), in some embodiments. In this regard, the graphical elements 530a with their displayed highlighting or hatching, in some embodiments, may be considered a first displayed path representing the first encircling path 529a, according to some embodiments. In some embodiments, the graphical elements 530a may be considered user-selected, or directly user-selected graphical elements (e.g., in order to select the corresponding transducers represented by such graphical elements), since they are overlapped by or intersected by the user-defined first encircling path 529a, according to some embodiments. In some embodiments, a user-defined encircling path, such as first encircling path 529a, may be displayed in addition to the displayed representation of the encircling path, such as the displayed path represented by the hatched ring of graphical elements 530a. In this regard, for example, first encircling path 529a may be displayed distinctly from the displayed plurality of graphical elements 502. In the example of FIG. 50, the first encircling path 529a may be displayed as the dark hand-drawn line shown in FIG. 5O, which is graphically or visually distinct (e.g., visually stands out) from the graphical elements 502. However, other manners of distinctly representing the encircling path 529a may be utilized in other embodiments.

In some embodiments associated with block 604c, the second encircling path 529b may be among a second group of at least some of the displayed plurality of graphical elements 502. In the example of FIG. 5O, such second group may be the graphical elements 530b (only two called out in FIG. 5O for purposes of clarity) with the internal pattern having vertical lines. Such graphical elements 530b may be displayed with such internal pattern having vertical lines in order to highlight them graphically to a user, such highlighting being a displayed representation of the second encircling path 529b, which might not be displayed and may merely represent the path traced by the corresponding user input received according to block 604 (or, e.g., block 604c or block 604cl), in some embodiments. In this regard, the graphical elements 530b with their displayed highlighting or hatching, in some embodiments, may be considered a second displayed path representing the second encircling path 529b, according to some embodiments. In some embodiments, the graphical elements 530b may be considered user-selected, or directly user-selected graphical elements (e.g., in order to select the corresponding transducers represented by such graphical elements), since they are overlapped by or intersected by the user-defined second encircling path 529b, according to some embodiments. In some embodiments, the second encircling path 529b, may be displayed in addition to the displayed representation of the second displayed path represented by the ring of hatched graphical elements 530b. In this regard, for example, second encircling path 529b may be displayed distinctly from the displayed plurality of graphical elements 502. In the example of FIG. 50, the second encircling path 529b may be displayed as the dark hand-drawn line shown in FIG. 5O, which is graphically or visually distinct (e.g., visually stands out) from the graphical elements 502. However, other manners of distinctly representing the encircling path 529b may be utilized in other embodiments.

According to some embodiments associated with block 604cl, at least a portion of the second encircling path may be located interior of the first encircling path. For example, as shown in FIG. 50, the second encircling path 529b is located interior of the first encircling path 529b. In at least some of such embodiments, the data processing device system 110, 310, may be configured at least by program instructions associated with block 606c in FIG. 6B to determine the first region of space 540 as being interior of the first encircling path (e.g., first encircling path 529a) and exterior the at least a portion of the second encircling path (e.g., second encircling path 529b) that is interior of the first encircling. In some embodiments, the at least the portion of the second encircling path is the entirety of the second encircling path, as is the case in the example of FIG. 5O, where the entirety of the second encircling path 529b is interior of the first encircling path 529a. With the first region of space 540 determined, the data processing device system 110, 310 may be configured to machine-select (e.g., according, in some embodiments, to program instructions associated with block 608 (or block 608a or 608b in some embodiments thereof)) the graphical elements 502 that are in such first region of space 540. In the example of FIG. 5O, such machine-selected graphical elements are illustrated by graphical elements 533, which have the interior grid (i.e., graph paper-like) pattern. The machine-selected graphical elements may correspond to the first transducer set selected per various embodiments of block 608, and the machine-selected graphical elements (e.g., illustrated by graphical elements 533) are distributed around the second encircling path (e.g., second encircling path 529b), in some embodiments. In the example of FIG. 50, the machine-selected graphical elements 533 may be considered a first selected graphical element set corresponding to a first transducer set selected according, in some embodiments, to block 608 (or block 608a or 608b in some embodiments thereof). In some embodiments, the transducers of the first transducer set are distributed around (or their corresponding graphical elements 533 are distributed around) the second encircling path 529b. In some embodiments, the second encircling path 529b encircles a second graphical element set (e.g., graphical elements 536 in the example of FIG. 5O) of the plurality of graphical elements 502.

Such a feature may allow the user to define a band of desired graphical elements by producing the first and second encircling paths 529a, 529b, where the graphical elements between such paths are machine- or automatically-selected, thereby allowing an efficient mechanism for selecting a targeted group of transducers, such that, for example, all hatched graphical elements in FIG. 5O (e.g., 530a, 530b, 533) may be selected for activation in some embodiments. By varying the positioning of the first and second encircling paths, the user is able to define the width or other shape characteristics of the graphical elements to be selected. While the example of FIG. 50 shows nested encircling paths 529a, 529b, other embodiments may have the user-defined paths be any other types of paths, including other paths (e.g., including open paths), that define a region of space between them, such that the graphical elements between such user-defined paths are machine-selected, e.g., according, in some embodiments, to program instructions associated with block 608 (or block 608a or 608b, in some embodiments thereof).

FIG. 5Q illustrates additional embodiments produced by methods 600. FIG. 5Q illustrates a simplified version of the graphical representation 500 for ease of discussion. The graphical representation of FIG. 5Q includes between graphical elements 504 (only one called out in FIG. 5Q) in addition to the transducer graphical elements 502 (only one called out in FIG. 5Q). Encircling path 529d is produced via the user input received per block 604, block 604b, or block 604b2, in some embodiments. As discussed above, encircling path 529d may or may not be visually presented to the user, but is shown in FIG. 5Q merely for purposes of clarity. In some embodiments, the definition of the encircling path 529d may indicate to the data processing device system 110, 310 that transducer graphical elements 530c (three called out in FIG. 5Q) and the corresponding between graphical elements 504c (three called out in FIG. 5Q) are to be user-selected. Such selection is indicated in FIG. 5Q using the internal vertical line pattern in the transducer graphical elements 530c and the corresponding between graphical elements 504c.

The encircling path 529d may then be used by the data processing device system 110, 310 to determine or define the first region of space 540 per block 606 or block 606a, in some embodiments. With the first region of space 540 defined, the data processing device system 110, 310 may select, as a machine-based selection, the between graphical elements 504d (three called out in FIG. 5Q) as those being directed inward (could be outward in some other embodiments) of the encircling path 529d (and inward of the user-selected graphical elements 530c in some embodiments). The selection of such between graphical elements 504d is illustrated in FIG. 5Q with the use of an internal horizontal line pattern in the corresponding graphical elements 504d. The selection of such between graphical elements 504d may then be used by the data processing device system 110, 310 to select the transducer graphical elements 535 (three called out in FIG. 5Q) on the other ends of the respective between graphical elements 504d as part of block 608 in some embodiments (or block 608a or 608b in some embodiments thereof). During activation per block 610, bipolar ablation may be performed by the respective transducer pairs associated with the selected between graphical elements 504d, according to some embodiments. According to some embodiments, the highlighting or hatching of graphical elements (internal horizontal and vertical line patterns in the case of FIG. 5Q) may be retained or modified after ablation to indicate to the user where a lesion has been formed, in some embodiments. It is noted that, in some embodiments, between graphical elements 504d may additionally or alternatively extend diagonally between various adjacent ones of the transducer graphical elements 535.

Turning now to FIG. 6C, some additional embodiments of block 606 will now be described. In this regard, the block 606′ in FIG. 6C may replace block 606 in FIG. 6B, according to some embodiments. Blocks 606a′, 606b′, and 606c′ in FIG. 6C may correspond to blocks 606a, 606b, and 606c in FIG. 6B, according to some embodiments, except that they refer to the determined positional relationship as a first determined positional relationship. Block 606′ may also differ from block 606 in that it additionally includes a determination of a second region of space per reference 606d′. Per block 606e′, the second region of space may be in a second determined positional relationship with respect to the encircling path, according to some embodiments. The second determined positional relationship may be the opposite of the first determined positional relationship. For instance, if the first determined positional relationship is interior of the encircling path, the second determined positional relationship may be exterior of the encircling path.

As described in more detail with respect to the example of FIG. 5R, at least some of such embodiments may be beneficial when it is desired that there be a machine selection of transducer graphical elements (or their associated transducers) on both sides of the encircling path. By selecting transducer graphical elements on both sides of the encircling path, a thick (multi-transducer-wide) ablation path may easily be selected, which may ensure an electrophysiological conduction block by the ablation.

For instance, according to some embodiments associated with FIG. 5R, a user may provide or indicate encircling path 529e via user input received by the data processing device system 110, 310 per block 604. In some embodiments, the formation of encircling path 529e may result in user-selected graphical elements 530d (three called out in FIG. 5R) that are intersected by the encircling path 529e, according to some embodiments. Per block 606′, the data processing device system 110, 310 may be configured to determine a first region of space 540a that is interior of the encircling path 529e (in some embodiments in which the first determined positional relationship per block 606a′, for example, is interior of the encircling path). Also, per block 606′, the data processing device system 110, 310 may be configured to determine a second region of space 540b that is exterior of the encircling path 529e (in some embodiments in which the second determined positional relationship per block 606e′, for example, is exterior of the encircling path 529e).

The data processing device system 110, 310 may then be configured by program instructions associated with block 608 (or block 608a or 608b, in some embodiments thereof) to cause, in response to reception of the user input indicating the encircling path 529e and in response to the determinations of the first and second regions of space 540a, 540b in the first and second determined positional relationships, respectively, with respect to the encircling path 529c, a machine-based selection of a first transducer set of the plurality of transducers, the machine-based selection selecting the first transducer set as collectively having corresponding graphical elements of the displayed plurality of graphical elements in both the first and second regions of space 540a, 540b. In some embodiments, the machine-based selection selects each transducer in the first transducer set as having at least part of a corresponding graphical element of the displayed plurality of graphical elements in the determined first region of space, in the determined second region of space, or in both the determined first and second regions of space. In the example of FIG. 5R, the first transducer set may include transducers whose corresponding transducer graphical elements 535a, 535b are in the first region of space 540a or the second region of space 540b, respectively. As can be seen in FIG. 5R, selecting transducer graphical elements on both sides of the encircling path 529e can ensure there are no ‘one-transducer-wide’ segments along the ablation path, according to some embodiments.

In some embodiments, it may be considered that a combination of block 606′ or block 606e′ and block 608 may result in a configuration of the data processing device system (110, 310), e.g., via selection instructions to cause, in response to reception of the user input per some embodiments of block 604 indicating an encircling path and in response to the determination per 606d′ and block 606e′ of the second region of space in the second determined positional relationship with respect to the encircling path, a machine-based selection of a second transducer set of the plurality of transducers (e.g., 220, 306, or 406 in some embodiments), the machine-based selection selecting each transducer in the second transducer set as having at least part of a corresponding graphical element of the displayed plurality of graphical elements in the determined second region of space. In this regard, e.g., block 608 may include a machine-based selection of a first transducer set having the first determined positional relationship (e.g., interior of the encircling path, in some embodiments) and a machine-based selection of a second transducer set having the second determined positional relationship (e.g., exterior of the encircling path, in some embodiments). For example, with respect to the example of FIG. 5R, the first transducer set may include transducers whose corresponding transducer graphical elements 535a in the first region of space 540a, and the second transducer set may include transducers whose corresponding transducer graphical elements 535b in the second region of space 540b, in some embodiments. In some of these embodiments, the activation instructions associated with block 610 may configure the data processing device system (e.g., 110, 310) to cause, via the input-output device system (e.g., 120, 320), activation of selected transducers of the plurality of transducers including each transducer in the first transducer set and each transducer in the second transducer set. In some embodiments, and as shown in FIG. 5R, for example, the selected transducers of the plurality of transducers including each transducer in the first transducer set and each transducer in the second transducer set may consist of some, but not all, of the plurality of transducers.

FIGS. 5S-1 and 5S-2 illustrate yet additional embodiments. In particular, while the above discussions have included embodiments where the formation of a path, such as an encircling path, is formed among a graphical representation of graphical elements, such as transducer graphical elements, some other embodiments allow the path or encircling path to be formed independently of the presence of such graphical elements, such as transducer graphical elements. For example, FIG. 5S-1 illustrates a simplified version of graphical representation 500 in a state in which a representation of a portion of a bodily cavity is represented as including a port 560 in the bodily cavity. In the state of FIG. 5S-1, which may be considered to occur at a first time, only a portion of a representation 300a of a transducer-based device is visible, such that only 16 transducer graphical elements of 64 total transducer graphical elements are viewable in such state, in this example. The other transducer graphical elements may be ‘off screen’ as the corresponding transducers are not sufficiently close to the port 560 in the state of FIG. 5S-1. In the state and at the first time represented by FIG. 5S-1, the user input may be received according to reception program instructions per block 604 to indicate the encircling path 529f around the graphical representation of the port 560. Per block 606, first region of space 540 may be determined as shown in FIG. 5S-1, according to some embodiments. In this regard, although the state of FIG. 5S-1 is referred to as occurring at a “first time” when the user input indicating the encircling path 529f is received, the state of FIG. 5S-1 may actually represent a small span of time including the time between reception of the user input and the determination of the first region of space 540, for purposes of clarity. However, the machine selection of transducer graphical elements 502 per block 608 (or block 608a or 608b in some embodiments thereof) need not occur until the transducer-based device moves in the direction 538 from the state of FIG. 5S-1 to the state of FIG. 5S-2, which may be considered to occur at a second time, where transducer graphical elements 502 are now located in the region of the graphical representation of the port 560. In this regard, the arrow used to illustrate the direction of movement 538 is merely for illustration purposes and would not be actually visually presented as part of the graphical representation illustrated by FIG. 5S-1, in some embodiments. In the state of FIG. 5S-2, which may be considered to occur at a second time after the first time represented by FIG. 5S-1, the machine-based selection per block 608 (or block 608a or 608b in some embodiments thereof) may occur, resulting in a selection of at least some of the hatched graphical elements 537 (three called out in FIG. 5S-2), according to some embodiments. In this regard, in some embodiments, reception instructions, which may be the same or similar to those associated with block 604, in some embodiments, may configure a data processing device system (e.g., 110, 310) to receive, via an input-output device system (e.g., 120, 320), movement information between the first time (e.g., the state of FIG. 5S-1) and the second time (e.g., the state of FIG. 5S-2), this movement information indicating movement of at least part of the first transducer set (e.g., represented as the transducers that correspond to those of the hatched transducer graphical elements 537 that are machine-selected in the state of FIG. 5S-2, and such transducer graphical elements are also shown in the state of FIG. 5S-1, but they are not hatched) into a particular region of the bodily cavity corresponding to the first region of space 540 by the second time. Such movement information may be produced by and provided from a catheter-device-location tracking system or navigation system, discussed above.

In some embodiments, all of the hatched transducer graphical elements 537 in FIG. 5S-2 are considered to be machine-selected per block 608. For instance, in some embodiments, the first transducer set that is machine-selected per block 608 includes transducers (or whose transducer graphical elements) that are not only entirely within first region of space 540, but also those that are in part within the first region of space 540. Those that are in part within the first region of space includes those that are intersected by the encircling path. Accordingly, the hatched transducer graphical elements 537 in FIG. 5S-2 may correspond to the first transducer set, where each hatched transducer graphical element 537 is at least in part in the first region of space 540. In this regard, in some embodiments, the first transducer set may include a particular transducer having a corresponding graphical element of the displayed plurality of graphical elements that is intersected by the encircling path (e.g., 529f).

In some embodiments, when a user produces an encircling path via user input, and transducers or their graphical elements are intersected by the encircling path, such intersecting transducers or graphical elements are considered to be user selected. On the other hand, in some embodiments, FIGS. 5S-1 and 5S-2 illustrate at least one case or situation where an encircling path (e.g., encircling path 529f) is user-produced, but transducers or their graphical elements that intersect the encircling path are considered to be machine-selected. For instance, in the state of FIG. 5S-1, the user produces encircling path 529f and, in such state, no graphical elements are intersected by the encircling path 529f. However, after movement of the transducer-based device from the state of FIG. 5S-1 to the state of FIG. 5S-2 per the direction of movement 538, transducer graphical elements are now in a position where some of them are intersected by the previously-produced encircling path 529f. Accordingly, at the time of FIG. 5S-2, the data processing device system (e.g., 110, 310) may perform the machine selection per block 608 to select not only the transducer graphical elements 502 that are entirely within the first region of space 540, but also those that are intersected by the encircling path 529f and, consequently, are only partially within the first region of space 540, in some embodiments, to form the machine-selected transducer set (or first transducer set) per block 608. Accordingly, depending on the embodiment, the machine-based selection per block 608 may or may not include transducers or transducer graphical elements that are partially within the first region of space 540 (e.g., are intersected by the encircling path). In some embodiments, the user input per some embodiments of block 604 includes (a) a user-based selection indicating a user-selected path (e.g., encircling path 529f), and the selection instructions associated with some embodiments of block 608 may be configured to cause, in response to the user-based selection, a particular machine-based selection of at least part of a selected graphical element set (e.g., represented by some of the hatched graphical elements 537 in FIG. 5S-2 that intersect the encircling path 529f) as along the user-selected path. In some embodiments in which the transducer-based device may be considered to be relatively stationary with respect to an adjacent tissue surface, and the formation of the encircling path intersects transducers or their graphical elements at the time of formation of the encircling path, those transducers may be considered user-selected, while the transducers or their graphical elements that are entirely within the first region of space and are not intersected by the encircling path may be considered machine-selected per block 608, according to some embodiments. In some embodiments, FIGS. 5J, 5K, 5L, 5M, 5N, 5O, 5P, 5Q, and 5R may be considered to represent some embodiments in which the transducer-based device may be considered to be relatively stationary, e.g., such that the user input received per some embodiments of block 604 indicating the encircling path is received according to the corresponding reception instructions in a state in which at least part of the first transducer set is in a particular region of the bodily cavity corresponding to the first region of space 540, according to some embodiments.

While some of the embodiments disclosed above are described with examples of cardiac mapping, the same or similar embodiments may be used for mapping other bodily organs, for example, gastric mapping, bladder mapping, arterial mapping and mapping of any lumen or cavity into which the devices of the present invention may be introduced.

While some of the embodiments disclosed above are described with examples of cardiac ablation, the same or similar embodiments may be used for ablating other bodily organs or any lumen or cavity into which the devices of the present invention may be introduced.

Subsets or combinations of various embodiments described above can provide further embodiments.

These and other changes can be made to the invention in light of the above-detailed description. In general, in the following claims, the terms used should not be construed to limit the invention to the specific embodiments disclosed in the specification and the claims but should be construed to include other transducer-based device systems including all medical treatment device systems and all medical diagnostic device systems in accordance with the claims. Accordingly, the invention is not limited by the disclosure, but instead its scope is to be determined entirely by the following claims.

What is claimed is:

1. A transducer-activation system comprising:
a data processing device system;
an input-output device system communicatively connected to the data processing device system; and
a memory device system communicatively connected to the data processing device system and storing a program executable by the data processing device system, the program comprising:
graphical representation instructions configured to cause display, via the input-output device system, of a graphical representation comprising a representation of at least a portion of a bodily cavity, the graphical representation comprising a plurality of graphical elements corresponding to a plurality of transducers positionable in the bodily cavity;
reception instructions configured to cause reception, via the input-output device system, of user input indicating an encircling path around at least a region of the representation of the at least the portion of the bodily cavity;
determination instructions configured to cause a determination of a first region of space in a determined positional relationship with respect to the encircling path, the determined positional relationship being interior of the encircling path or the determined positional relationship being exterior of the encircling path;
selection instructions configured to cause, in response to reception of the user input indicating the encircling path and in response to the determination of the first region of space in the determined positional relationship with respect to the encircling path, a machine-based selection of a first transducer set of the plurality of transducers, the machine-based selection selecting each transducer in the first transducer set as having at least part of a corresponding graphical element of the displayed plurality of graphical elements in the determined first region of space; and
activation instructions configured to cause, via the input-output device system, activation of selected transducers of the plurality of transducers including each transducer in the first transducer set.

2. The transducer-activation system of claim 1, wherein the graphical representation instructions are configured to cause display, via the input-output device system, of the encircling path among the at least some of the displayed plurality of graphical elements.

3. The transducer-activation system of claim 1, wherein the graphical representation instructions are configured to cause display, via the input-output device system, of the encircling path around the at least the region of the representation of the at least the portion of the bodily cavity.

4. The transducer-activation system of claim 1, wherein the graphical representation instructions are configured to cause display, via the input-output device system, of the encircling path distinctly from the displayed plurality of graphical elements.

5. The transducer-activation system of claim 1, wherein the first transducer set includes a particular transducer having a corresponding graphical element of the displayed plurality of graphical elements that is intersected by the encircling path.

6. The transducer-activation system of claim 1, wherein the selection instructions are configured to cause a selection indicating a selected graphical element set from the displayed plurality of graphical elements, the selected graphical element set corresponding to the first transducer set of the plurality of transducers.

7. The transducer-activation system of claim 6,
wherein the machine-based selection includes a first machine-based selection of a first graphical element of the selected graphical element set, the first graphical element corresponding to a first transducer in the first transducer set, the first graphical element selected according to the first machine-based selection as a first particular one of the plurality of graphical elements located closest to a portion of the encircling path among all graphical elements of the plurality of graphical elements located at least in part in the first region of space, and
wherein the machine-based selection includes a second machine-based selection of a second graphical element of the selected graphical element set, the second graphical element selected according to the second machine-based selection as a second particular one of the plurality of graphical elements located closest, besides the first graphical element, to the portion of the encircling path, among all graphical elements of the plurality of graphical elements that are in the first region of space and spaced from the encircling path, the second graphical element spaced from the encircling path, and the second graphical element corresponding to a second transducer in the first transducer set, the second transducer other than the first transducer.

8. The transducer-activation system of claim 7, wherein the first transducer and the second transducer are adjacent transducers of the plurality of transducers.

9. The transducer-activation system of claim 7, wherein the first graphical element selected according to the first machine-based selection is spaced from the encircling path.

10. The transducer-activation system of claim 7, wherein the first graphical element selected according to the first machine-based selection is intersected by the portion of the encircling path.

11. The transducer-activation system of claim 1,
wherein the machine-based selection includes a first machine-based selection of a first group of graphical elements of the plurality of graphical elements, each graphical element in the first group of graphical elements of the plurality of graphical elements corresponding to a respective transducer in a first group of transducers in the first transducer set, each graphical element in the first group of graphical elements of the plurality of graphical elements selected according to the first machine-based selection as a particular one of the plurality of graphical elements located in the first region of space closest to a respective portion of the encircling path among all graphical elements of the plurality of graphical elements located in or located at least in part in the first region of space, and
wherein the machine-based selection includes a second machine-based selection of a second group of graphical elements of the plurality of graphical elements, each graphical element in the second group of graphical elements of the plurality of graphical elements selected according to the second machine-based selection as a particular one of the plurality of graphical elements in the first region of space other than any graphical element in the first group of graphical elements of the plurality of graphical elements, each graphical element in the second group of graphical elements of the plurality of graphical elements corresponding to a respective transducer in a second group of transducers in the first transducer set.

12. The transducer-activation system of claim 11, wherein each graphical element in the second group of graphical elements of the plurality of graphical elements is adjacent each of at least one graphical element in the first group of graphical elements of the plurality of graphical elements.

13. The transducer-activation system of claim 11, wherein each transducer in the second group of transducers is adjacent each of at least one other transducer in the second group of transducers.

14. The transducer-activation system of claim 1, wherein the machine-based selection includes a machine-based selection of a particular group of graphical elements of the plurality of graphical elements, each graphical element in the selected particular group of graphical elements of the plurality of graphical elements corresponding to a respective transducer in the first transducer set, and each graphical element of at least one particular graphical element in the selected particular group of graphical elements of the plurality of graphical elements adjacent each graphical element of a respective set of at least three graphical elements in the selected particular group of graphical elements of the plurality of graphical elements.

15. The transducer-activation system of claim 14, wherein each graphical element in each respective set of at least three graphical elements in the selected particular group of graphical elements of the plurality of graphical elements is adjacent each other graphical element in the respective set of at least three graphical elements in the selected particular group of graphical elements of the plurality of graphical elements.

16. The transducer-activation system of claim 1, wherein the machine-based selection includes a machine-based selection of a particular group of graphical elements of the plurality of graphical elements, each graphical element in the selected particular group of graphical elements of the plurality of graphical elements corresponding to a respective transducer in the first transducer set, and each graphical element in the selected particular group of graphical elements of the plurality of graphical elements adjacent each graphical element of a respective set of at least three graphical elements in the selected particular group of graphical elements of the plurality of graphical elements.

17. The transducer-activation system of claim 16, wherein at least one graphical element in a first respective set of at least three graphical elements in the selected particular group of graphical elements of the plurality of graphical elements is other than any graphical element in a second respective set of at least three graphical elements in the selected particular group of graphical elements of the plurality of graphical elements.

18. The transducer-activation system of claim 16, wherein each graphical element in a first respective set of at least three graphical elements in the selected particular group of graphical elements of the plurality of graphical elements is other than any graphical element in a second respective set of at least three graphical elements in the selected particular group of graphical elements of the plurality of graphical elements.

19. The transducer-activation system of claim 14, wherein at least one graphical element of the at least one particular graphical element in the selected particular group of graphical elements of the plurality of graphical elements is located closest to a portion of the encircling path among all graphical elements of the plurality of graphical elements in the first region of space.

20. The transducer-activation system of claim 14, wherein the selected particular group of graphical elements of the plurality of graphical elements consists of some, but not all, of the graphical elements of the plurality of graphical elements in the first region of space.

21. The transducer-activation system of claim 14, wherein the selected particular group of graphical elements of the plurality of graphical elements includes all of the graphical elements of the plurality of graphical elements in the first region of space.

22. The transducer-activation system of claim 1, wherein the machine-based selection includes a machine-based selection of a particular group of graphical elements of the plurality of graphical elements, each graphical element in the selected particular group of graphical elements of the plurality of graphical elements corresponding to a respective transducer in the first transducer set, and each graphical element of at least one particular graphical element in the selected particular group of graphical elements of the plurality of graphical elements adjacent each graphical element of a respective set of at least five graphical elements in the selected particular group of graphical elements of the plurality of graphical elements.

23. The transducer-activation system of claim 1, wherein the encircling path defines at least three non-colinear locations in the graphical representation.

24. The transducer-activation system of claim 1, wherein the activation of each transducer in the first transducer set is configured to cause tissue ablation.

25. The transducer-activation system of claim 1,
wherein the user input indicating the encircling path is received according to the reception instructions at a first time,
wherein the machine-based selection occurs at a second time after the first time, and
wherein the reception instructions are configured to cause reception, via the input-output device system, of movement information received between the first time and the second time, the movement information indicating movement of at least part of the first transducer set into a particular region of the bodily cavity corresponding to the first region of space by the second time.

26. The transducer-activation system of claim 1, wherein the user input indicating the encircling path is received according to the reception instructions in a state in which at least part of the first transducer set is in a particular region of the bodily cavity corresponding to the first region of space.
